(19) United States
(12) Patent Application Publication
(10) Pub. No.: US 2014/0107316 A1
(43) Pub. Date: Apr. 17, 2014

(54) DRUG DELIVERY CONJUGATES CONTAINING UNNATURAL AMINO ACIDS AND METHODS FOR USING

(71) Applicant: Endocyte, Inc., (US)

(72) Inventors: Iontcho R. VLAHOV, West Lafayette, IN (US); Christopher P. LEAMON, West Lafayette, IN (US)

(73) Assignee: ENDOCYTE, INC., West Lafayette, IN (US)

(21) Appl. No.: 13/837,539

(22) Filed: Mar. 15, 2013

Related U.S. Application Data
(60) Provisional application No. 61/714,565, filed on Oct. 16, 2012.
(60) Provisional application No. 61/790,234, filed on Mar. 15, 2013.

(51) Int. Cl. A61K 47/48

(52) U.S. Cl. CPC A61K 47/48338
USPC 530/329; 530/330

(57) ABSTRACT

Described herein are drug delivery conjugates for targeted therapy. In particular, described herein are drug delivery conjugates that include polyvalent linkers comprising one or more unnatural amino acids.

### CROSS-REFERENCE TO RELATED APPLICATIONS

The present application claims the benefit, under 35 U.S.C. §119(e), of U.S. Provisional Application No. 61/714,565, which was filed Oct. 16, 2012 and U.S. Provisional Application 61/790,234, which was filed Mar. 15, 2013, the entirety of each of the disclosures of which are hereby incorporated herein by reference.

### TECHNICAL FIELD

The invention described herein pertains to drug delivery conjugates for targeted therapy. In particular, the invention described herein pertains to drug delivery conjugates that include polyvalent linkers comprising one or more unnatural amino acids.

### BACKGROUND AND SUMMARY OF THE INVENTION

It has been discovered that drug delivery conjugates that include polyvalent linkers formed from one or more unnatural amino acids are efficacious in treating pathogenic cell populations, including but not limited to cancer, and for treating inflammation, including but not limited to arthritis.

In one illustrative embodiment of the invention, compounds of the formula

B-L-D_{X}

are described herein.

In another embodiment, pharmaceutical compositions containing one or more of the compounds are also described herein. In one aspect, the compositions include a therapeutically effective amount of the one or more compounds for treating a patient with cancer, inflammation, and the like. It is to be understood that the compositions may include other component and/or ingredients, including, but not limited to, other therapeutically active compounds, and/or one or more carriers, diluents, excipients, and the like. In another embodiment, methods for using the compounds and pharmaceutical compositions for treating patients with cancer, inflammation, and the like are also described herein. In one aspect, the methods include the step of administering one or more of the compounds and/or compositions described herein to a patient with cancer, inflammation, and the like. In another aspect, the methods include administering a therapeutically effective amount of the one or more compounds and/or compositions described herein for treating patients with cancer, inflammation, and the like. In another embodiment, uses of the compounds and compositions in the manufacture of a medicament for treating patients with cancer, inflammation, and the like are also described herein. In one aspect, the medicaments include a therapeutically effective amount of the one or more compounds and/or compositions for treating a patient with cancer, inflammation, and the like.

### BRIEF DESCRIPTION OF THE DRAWINGS

FIG. 1A shows alone in vivo activity of EC1456 against KB tumors in nu/nu mice dosed at 1 μmol/kg three times per week (M/W/F) (TIW) for two consecutive weeks (), compared to EC1456 co-dosed with EC0923 at 100 μmol/kg (▴), and untreated (PBS) controls (▪). Dotted vertical line represents the day of final dose. FIG. 1B shows that EC1456 did not result in any observable gross toxicity as determined by animal body weight.

FIG. 2A shows the activity of EC1456 Against Established Subcutaneous MDA-MB-231 Tumors. Animals bearing s.c. MDA-MB-231 tumors (94-145 mm³) were treated i.v. starting on Day 17 with 2 μmmol/kg (panel A) of EC1456 (), three times per week (M/W/F) for a 2 week period compared to untreated animals (▪), as shown in FIG. 2A. N=5 animals per cohort. Dotted vertical line=day of final dose. FIG. 2B shows that EC1456 did not cause gross whole animal toxicity as determined by % weight change.

FIG. 3A shows the activity of EC1456 in animals bearing s.c. KB-CR tumors (98-148 mm3), where EC1456 was administered i.v. starting on Day 6 with 2 μmol/kg (), three times per week (M/W/F) for a 2 week period, or with 3 mg/kg of cisplatin (▴), twice per week (T/Th) for a 2 week period, and compared to untreated controls (▪), N=5 animals per cohort. Dotted vertical line=day of final dosing day. FIG. 3B shows that EC1456 did not exhibit significant host animal toxicity, and that cisplatin did exhibit significant host animal toxicity.

FIG. 4A shows the activity of EC1663 in animals bearing s.c. KB tumors, where EC1663 was administered i.v. starting on Day 7 with 0.5 μmol/kg (▴), three times per week (M/W/F) for a 2 week period, for a 2 week period, and compared to untreated controls (), N=5 animals per cohort. Dotted vertical line=day of final dosing day. FIG. 4B shows that EC1663 did not exhibit significant host animal toxicity.

### DETAILED DESCRIPTION

Several illustrative embodiments of the invention are described by the following clauses:

A compound of the formula BLD_{X}, or a pharmaceutically acceptable salt thereof, wherein B is a cell surface receptor targeting ligand, D is in each instance an independently selected drug, x is an integer selected from 1, 2, 3, 4 and 5; and L is a releasable polyvalent linker comprising one or more unnatural amino acids; and where B is covalently attached to L, and L is covalently attached to each of D; and

where that the compound is not of the formula

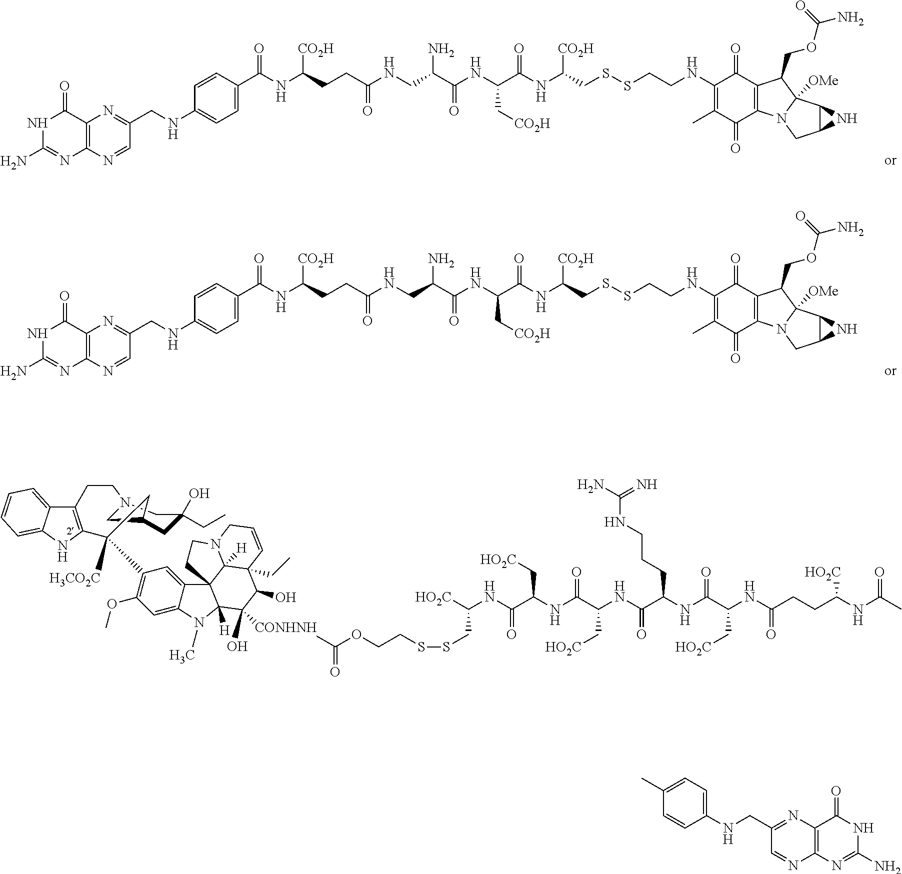

or a pharmaceutically acceptable salt thereof.

The compound of the preceding clause wherein at least one unnatural amino acid has the D-configuration.

The compound of any one of the preceding clauses wherein at least one unnatural amino acid is selected from D-alanine, D-aspartic acid, D-asparagine, D-cysteine, D-glutamic acid, D-phenylalanine, D-histidine, D-isoleucine, D-lysine, D-leucine, D-methionine, D-proline, D-glutamine, D-arginine, D-serine, D-threonine, D-valine, D-tryptophan, D-tyrosine, and D-ornithine, or a derivative thereof.

The compound of any one of the preceding clauses wherein at least one unnatural amino acid is selected from D-aspartic acid, D-asparagine, D-cysteine, D-glutamic acid, D-histidine, D-lysine, D-methionine, D-glutamine, D-arginine, D-serine, D-threonine, D-tryptophan, D-tyrosine, and D-ornithine, or a derivative thereof.

The compound of any one of the preceding clauses wherein at least one unnatural amino acid is selected from D-aspartic acid, D-asparagine, D-cysteine, D-glutamic acid, D-histidine, D-lysine, D-glutamine, D-arginine, D-serine, D-threonine, D-tryptophan, and D-ornithine, or a derivative thereof.

The compound of any one of the preceding clauses wherein at least one unnatural amino acid is selected from D-aspartic acid, D-cysteine, D-glutamic acid, D-lysine, D-arginine, D-serine, and D-ornithine, or a derivative thereof.

The compound of any one of the preceding clauses wherein L comprises two or more unnatural amino acids.

The compound of any one of the preceding clauses wherein L comprises three or more unnatural amino acids.

The compound of any one of the preceding clauses wherein L comprises four or more unnatural amino acids.

The compound of any one of the preceding clauses wherein L further comprises one or more disulfides.

The compound of any one of the preceding clauses wherein at least one disulfide comprises D-cysteinyl.

The compound of any one of the preceding clauses wherein L further comprises one or more divalent hydrophilic radicals.

The compound of any one of the preceding clauses wherein L further comprises two or more divalent hydrophilic radicals.

The compound of any one of the preceding clauses wherein L further comprises three or more divalent hydrophilic radicals.

The compound of any one of the preceding clauses wherein L further comprises four or more divalent hydrophilic radicals.

The compound of any one of the preceding clauses wherein L further comprises one or more divalent polyoxy radicals.

The compound of any one of the preceding clauses wherein L further comprises two or more divalent polyoxy radicals.

The compound of any one of the preceding clauses wherein L further comprises three or more divalent polyoxy radicals.

The compound of any one of the preceding clauses wherein L further comprises four or more divalent polyoxy radicals.

The compound of any one of the preceding clauses wherein L further comprises one or more divalent polyhydroxy radicals.

The compound of any one of the preceding clauses wherein L further comprises two or more divalent polyhydroxy radicals.

The compound of any one of the preceding clauses wherein L further comprises three or more divalent polyhydroxy radicals.

The compound of any one of the preceding clauses wherein L further comprises four or more divalent polyhydroxy radicals.

The compound of any one of the preceding clauses wherein at least one unnatural amino acid comprises a polyhydroxy radical.

The compound of any one of the preceding clauses wherein at least two unnatural amino acids comprise a polyhydroxy radical.

The compound of any one of the preceding clauses wherein at least three unnatural amino acids comprise a polyhydroxy radical.

The compound of any one of the preceding clauses wherein at least four unnatural amino acids comprise a polyhydroxy radical.

The compound of any one of the preceding clauses wherein at least one of the polyhydroxy radicals is of the formula

CH₂—(CH(OH))ₙ—CH₂—OH

where n is selected from 1, 2, 3, 4, 5, and 6.

The compound of any one of the preceding clauses wherein n is selected from 1, 2, 3, and 4.

The compound of any one of the preceding clauses wherein n is selected from 3 and 4.

The compound of any one of the preceding clauses wherein L comprises a divalent polyglutamic acid radical, where at least one glutamic acid forms an amide with an aminopolyhydroxy radical.

The compound of any one of the preceding clauses wherein L comprises a divalent polyglutamic acid radical, where at least two glutamic acids form an amide with an aminopolyhydroxy radical.

The compound of any one of the preceding clauses wherein L comprises a divalent polyglutamic acid radical, where at least three glutamic acids form an amide with an aminopolyhydroxy radical.

The compound of any one of the preceding clauses wherein L comprises a divalent polyglutamic acid radical, where at least four glutamic acids form an amide with an aminopolyhydroxy radical.

The compound of any one of the preceding clauses wherein L comprises a divalent poly(D-glutamic acid) radical, where at least one glutamic acid forms an amide with an aminopolyhydroxy radical.

The compound of any one of the preceding clauses wherein L comprises a divalent poly(D-glutamic acid) radical, where at least two glutamic acids form an amide with an aminopolyhydroxy radical.

The compound of any one of the preceding clauses wherein L comprises a divalent poly(D-glutamic acid) radical, where at least three glutamic acids form an amide with an aminopolyhydroxy radical.

The compound of any one of the preceding clauses wherein L comprises a divalent poly(D-glutamic acid) radical, where at least four glutamic acids form an amide with an aminopolyhydroxy radical.

The compound of any one of the preceding clauses wherein at least one of the aminopolyhydroxy radicals is of the formula

NH—CH₂—(CH(OH))ₘ—CH₂—OH

where m is selected from 1, 2, 3, 4, 5, and 6.

The compound of any one of the preceding clauses wherein L comprises a divalent radical of the formula

S—CH₂CH₂—O—C(O).

The compound of any one of the preceding clauses wherein L comprises a divalent radical of the formula

S—S—CH₂CH₂—O—C(O).

The compound of any one of the preceding clauses wherein m is selected from 1, 2, 3, and 4.

The compound of any one of the preceding clauses wherein m is selected from 3 and 4.

The compound of any one of the preceding clauses wherein B is a folate receptor binding moiety.

The compound of any one of the preceding clauses wherein B is a folate.

The compound of any one of the preceding clauses wherein B is a folate comprising D-glutamyl.

The compound of any one of the preceding clauses wherein B is folate.

The compound of any one of the preceding clauses wherein B is an unnatural folate radical of the formula

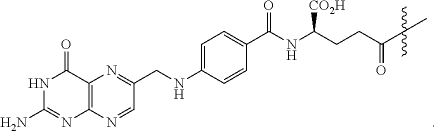

The compound of any one of the preceding clauses wherein x is 3.

The compound of any one of the preceding clauses wherein x is 2.

The compound of any one of the preceding clauses wherein x is 1.

The compound of any one of the preceding clauses wherein at least one D is a cytotoxic agent.

The compound of any one of the preceding clauses wherein at least one D is a cancer treating agent.

The compound of any one of the preceding clauses wherein at least one D is an anti-inflammatory agent.

The compound of any one of the preceding clauses wherein at least one D is a vinca alkaloid.

The compound of any one of the preceding clauses wherein at least one D is desacetylvinblastine monohydrazide.

The compound of any one of the preceding clauses wherein at least one D is a tubulysin.

The compound of any one of the preceding clauses wherein at least one D is tubulysin A.

The compound of any one of the preceding clauses wherein at least one D is tubulysin B.

The compound of any one of the preceding clauses wherein at least one D is tubulysin A hydrazide.

The compound of any one of the preceding clauses wherein at least one D is tubulysin B hydrazide.

The compound of any one of the preceding clauses wherein at least one D is an aminopterin.

The compound of any one of the preceding clauses wherein B-L is a radical of the formula

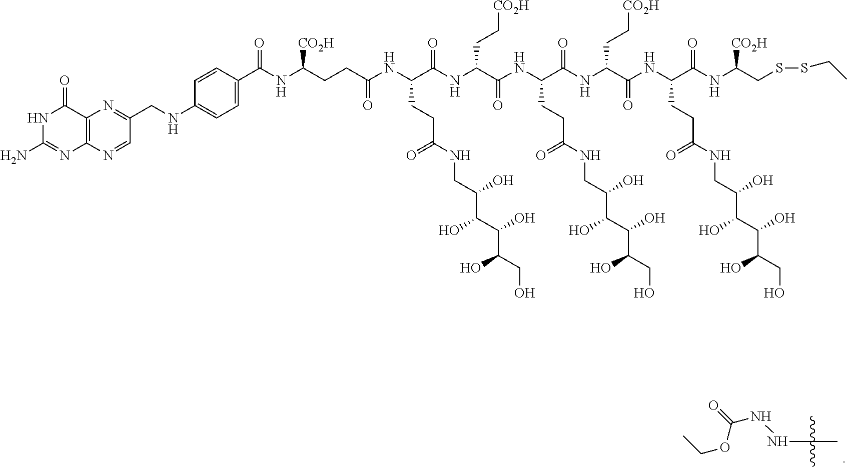

The compound of any one of the preceding clauses wherein B-L is a radical of the formula

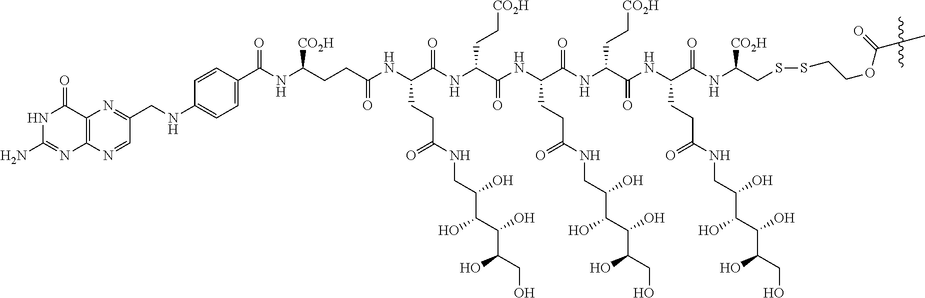

The compound of any one of the preceding clauses wherein at least one D is a radical of the formula

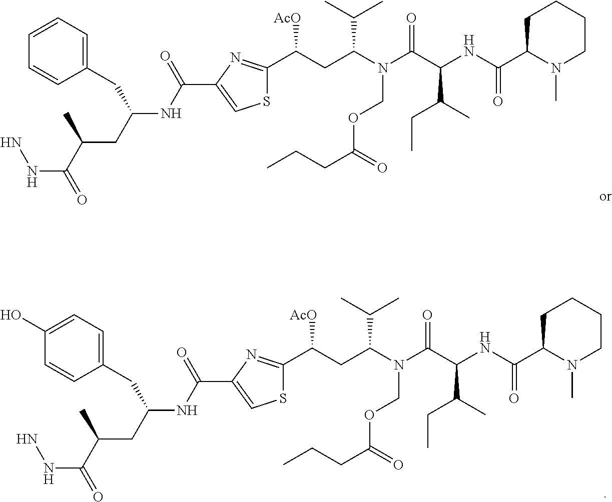

The compound of any one of the preceding clauses wherein at least one D is a radical of the formula

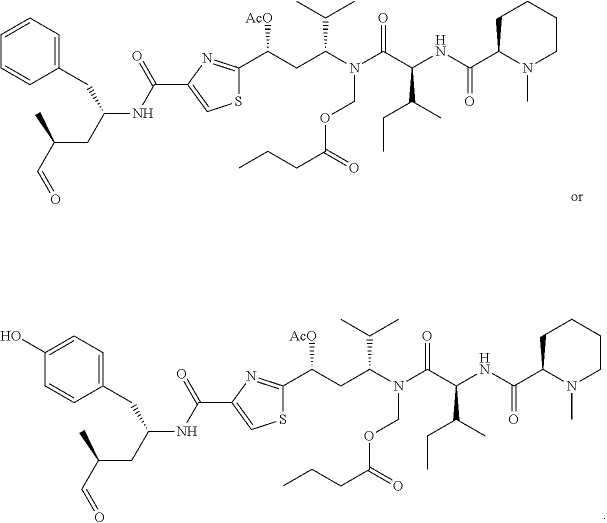

The compound of any one of the preceding clauses wherein at least one D is a radical of the formula

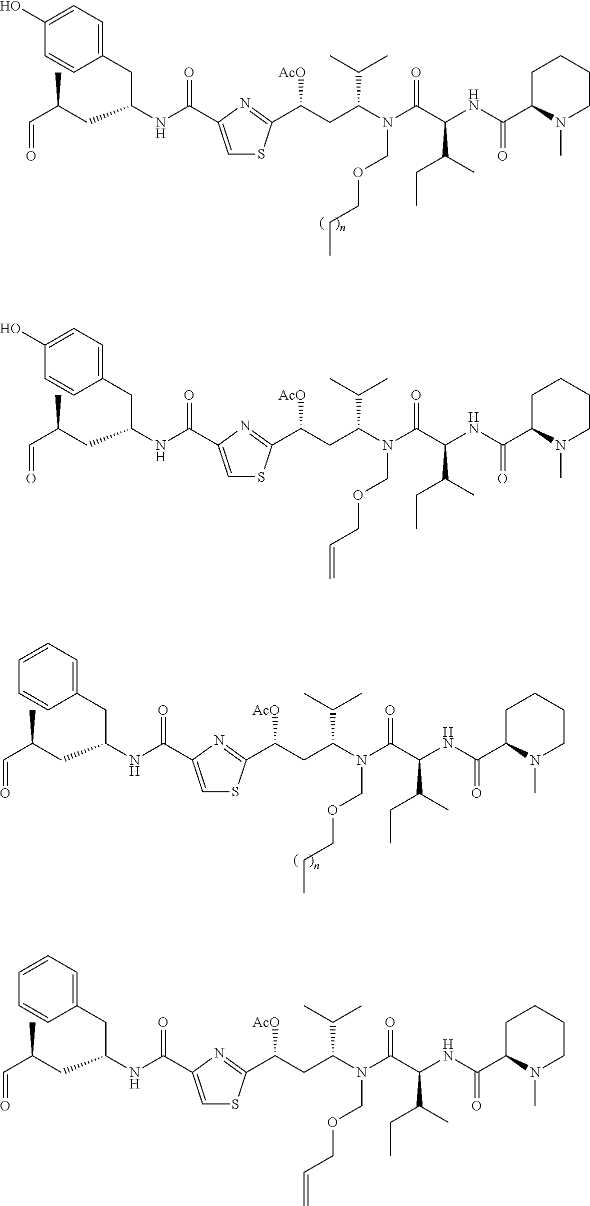

where n=1, 2, 3, 4, 5, or 6, or alternatively, n=1, 2, or 3, or alternatively, n=2 or 3.

The compound of any one of the preceding clauses wherein at least one D is a radical of the formula

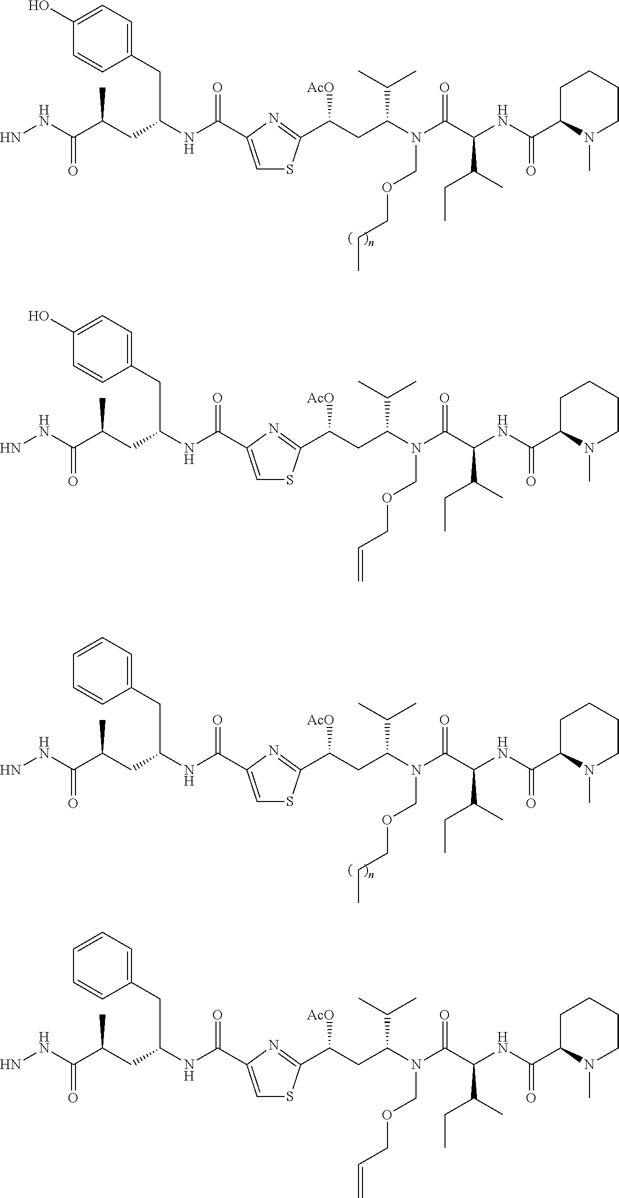

where n=1, 2, 3, 4, 5, or 6, or alternatively, n=1, 2, or 3, or alternatively, n=2 or 3.

The compound of any one of the preceding clauses wherein at least one D is a radical of the formula

The compound of any one of the preceding clauses wherein at least one D is a radical of the formula

The compound of any one of the preceding clauses wherein at least one D is a radical of the formula

The compound of any one of the preceding clauses wherein at least one D is a radical of the formula

The compound of any one of the preceding clauses wherein the compound is of the formula EC1456

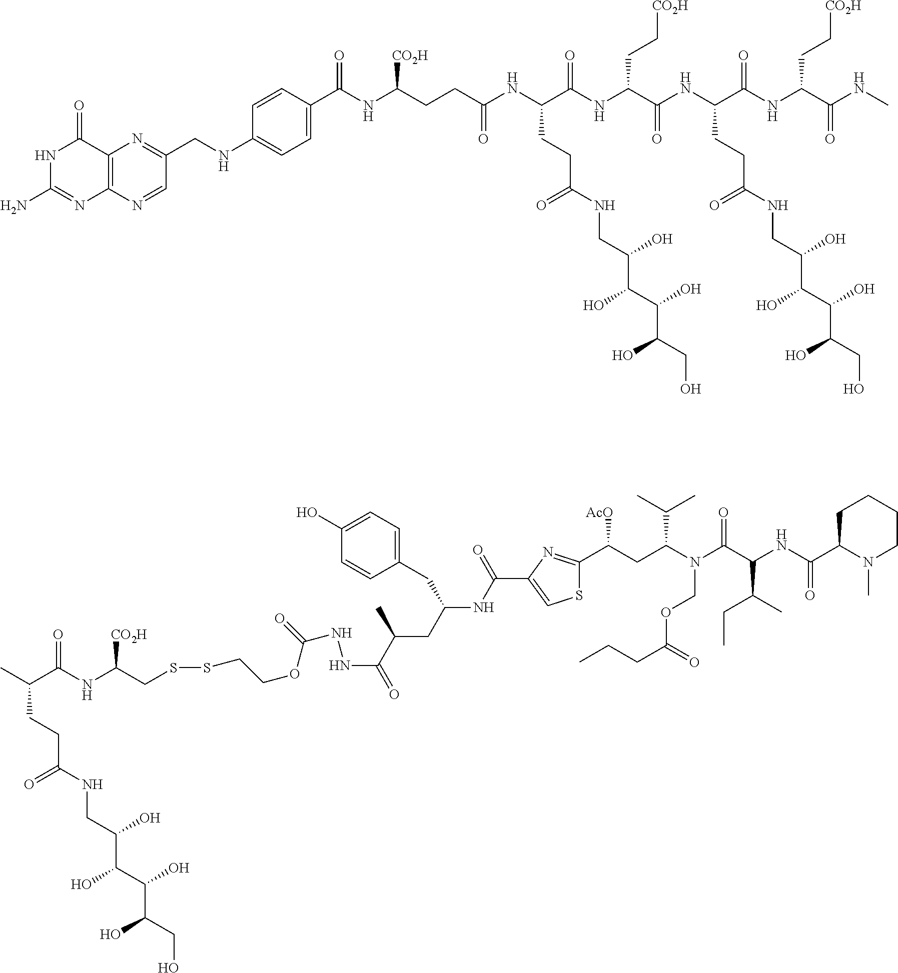

or a pharmaceutically acceptable salt thereof.

The compound of any one of the preceding clauses wherein the compound is not of the formula

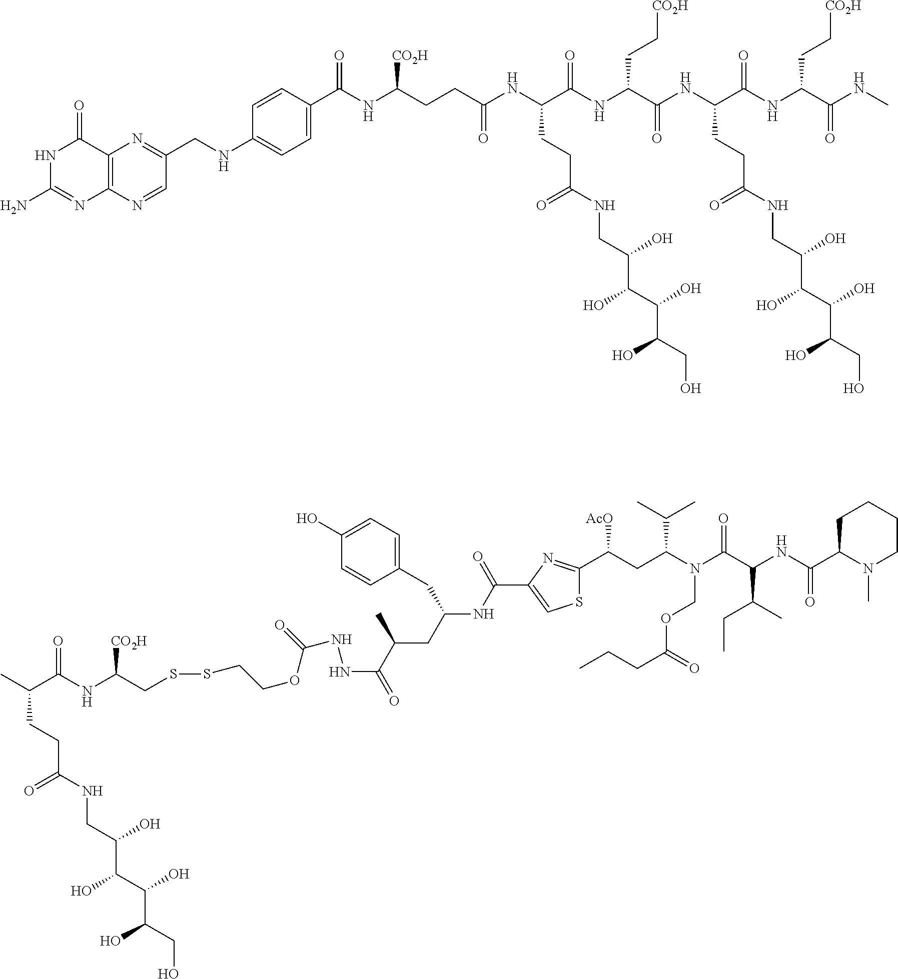

or a pharmaceutically acceptable salt thereof.

The compound of any one of the preceding clauses wherein the compound is of the formula EC1496

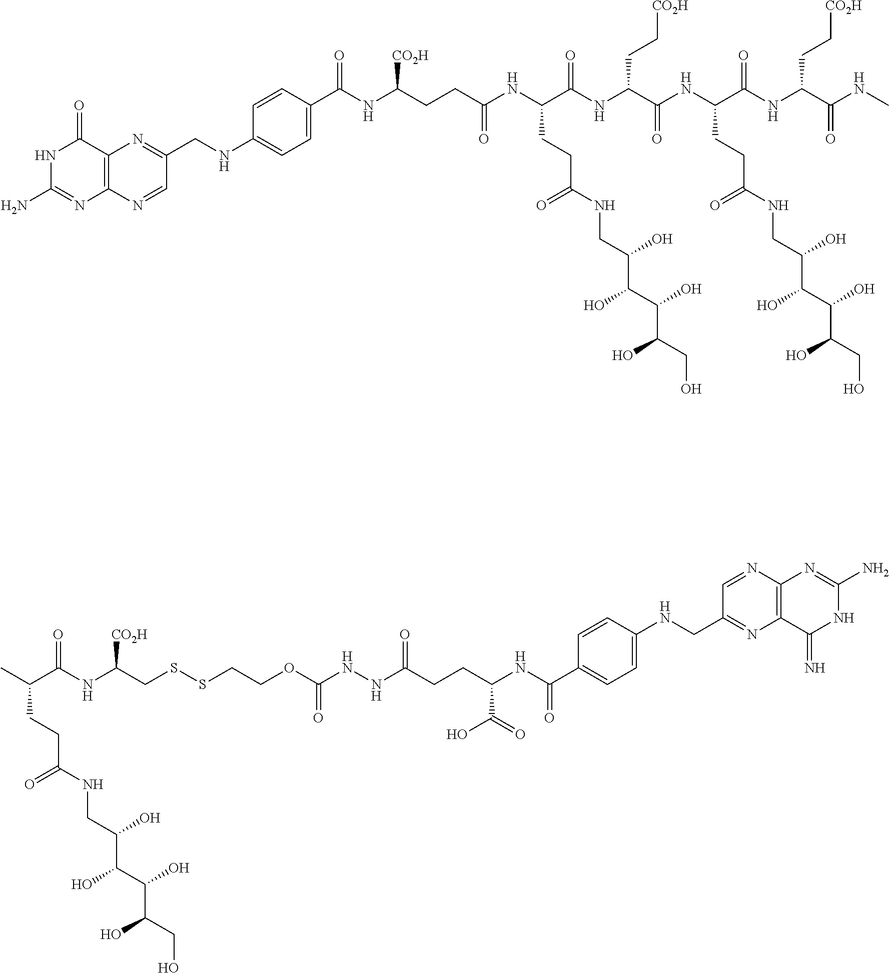

or a pharmaceutically acceptable salt thereof.

The compound of any one of the preceding clauses wherein the compound is not of the formula

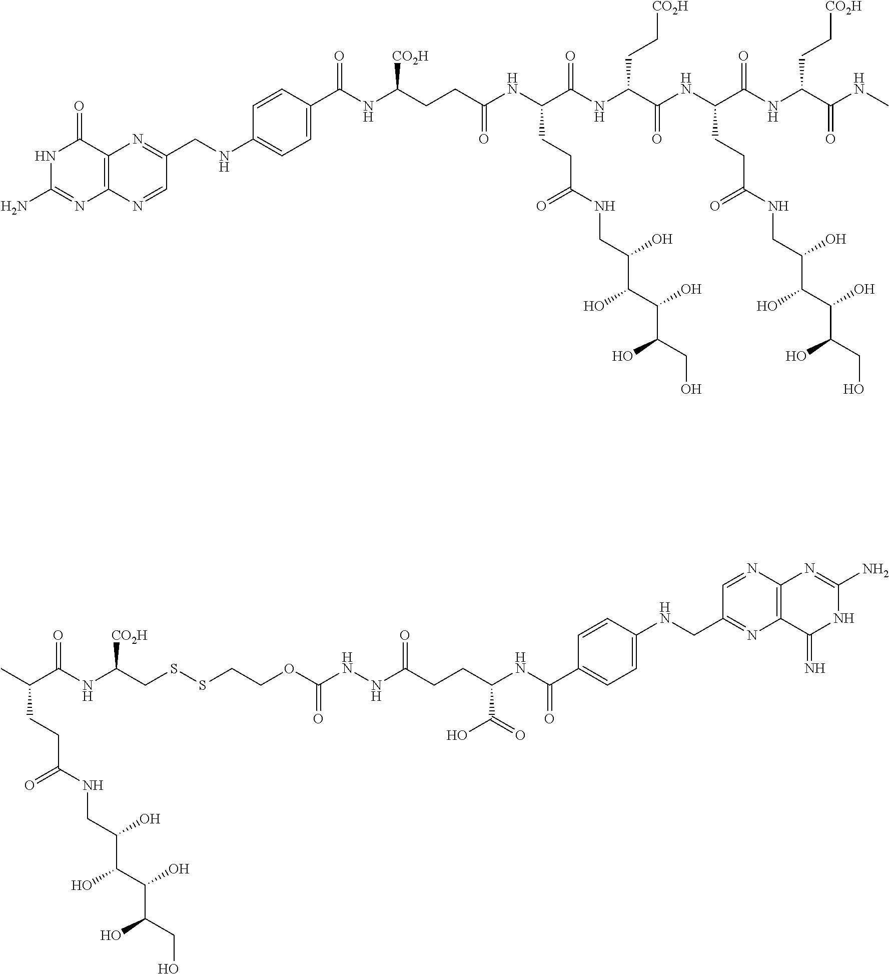

or a pharmaceutically acceptable salt thereof.

The compound of any one of the preceding clauses wherein the compound is more stable in plasma than the corresponding compound having fewer unnatural amino acids.

The compound of any one of the preceding clauses wherein the compound is more stable in vivo than the corresponding compound having fewer unnatural amino acids.

A pharmaceutical composition comprising a compound of any one of the preceding clauses in combination with one or more carriers, diluents, or excipients, or a combination thereof.

A unit dose or unit dosage form composition comprising a therapeutically effective amount of one or more compounds of any one of the preceding clauses, optionally in combination with one or more carriers, diluents, or excipients, or a combination thereof.

A method for treating cancer or inflammation in a host animal, the method comprising the step of administering to the host animal a composition comprising a therapeutically effective amount of one or more compounds of any one of the preceding clauses; or a pharmaceutical composition comprising one or more compounds of any one of the preceding clauses, optionally further comprising one or more carriers, diluents, or excipients, or a combination thereof.

A method for treating drug resistant cancer in a host animal, the method comprising the step of administering to the host animal a composition comprising a therapeutically effective amount of one or more compounds of any one of the preceding clauses; or a pharmaceutical composition comprising one or more compounds of any one of the preceding clauses, optionally further comprising one or more carriers, diluents, or excipients, or a combination thereof.

The method of any one of the preceding clauses wherein the cancer is an ovarian cancer.

The method of any one of the preceding clauses wherein the cancer is a drug resistant ovarian cancer.

The method of any one of the preceding clauses wherein the cancer is a platinum resistant ovarian cancer, such as NCI/ADR-RES or NCI/ADR-RES related ovarian cancer.

The method of any one of the preceding clauses wherein the cancer is a platinum resistant ovarian cancer, such as IGROVCDDP or IGROVCDDP related ovarian cancer.

The method of any one of the preceding clauses wherein the cancer is a cisplatin resistant ovarian cancer.

The method of any one of the preceding clauses wherein the cancer is a breast cancer.

The method of any one of the preceding clauses wherein the cancer is a drug resistant breast cancer.

The method of any one of the preceding clauses wherein the cancer is a triple negative breast cancer, such as MDA-MB-231 or MDA-MB-231 related breast cancer.

An intermediate for preparing a compound of claim 1 of the formula

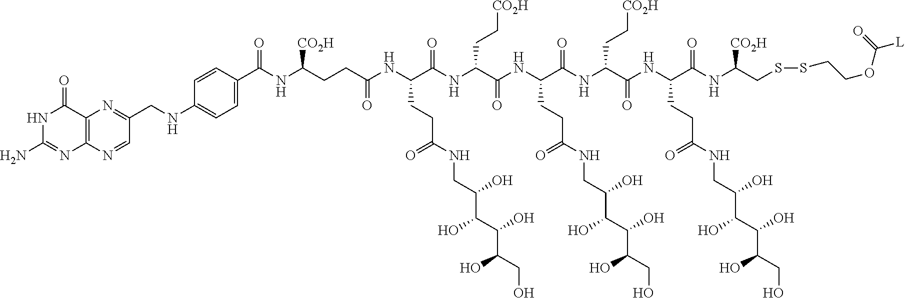

or a pharmaceutically acceptable salt thereof, wherein L is a leaving group.

An intermediate for preparing a compound of claim 1 of the formula

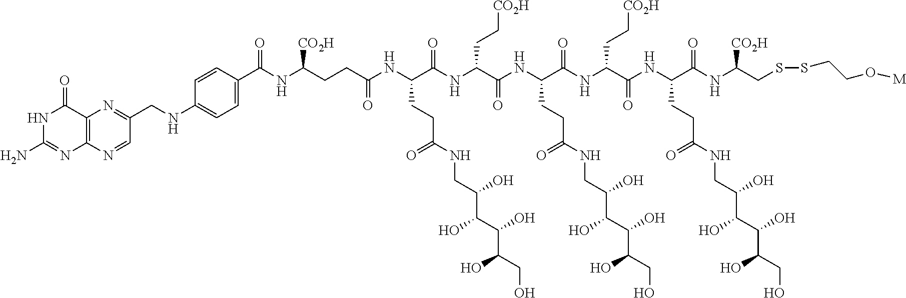

or a pharmaceutically acceptable salt thereof, wherein M is hydrogen or a cation.

An intermediate for preparing a compound of claim 1 of the formula

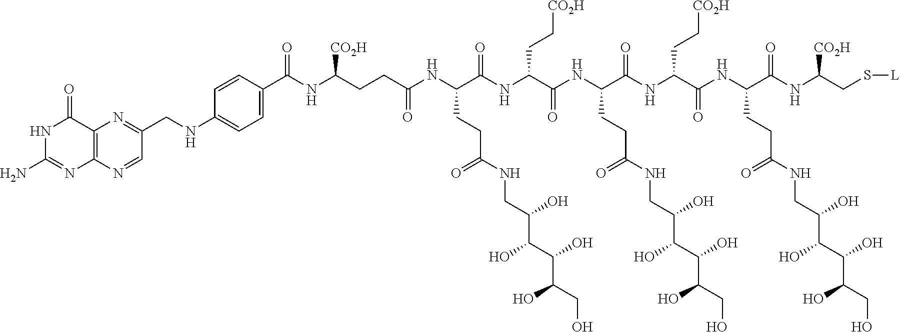

or a pharmaceutically acceptable salt thereof, wherein L is a leaving group.

The compounds described herein can be used for both human clinical medicine and veterinary applications. Thus, the host animal harboring the population of pathogenic cells and treated with the compounds described herein can be human or, in the case of veterinary applications, can be a laboratory, agricultural, domestic, or wild animal. The present invention can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

The invention is applicable to populations of pathogenic cells that cause a variety of pathologies in these host animals. In accordance with the invention “pathogenic cells” means cancer cells, infectious agents such as bacteria and viruses, bacteria- or virus-infected cells, activated macrophages capable of causing a disease state, and any other type of pathogenic cells that uniquely express, preferentially express, or overexpress vitamin receptors or receptors that bind analogs or derivatives of vitamins. Pathogenic cells can also include any cells causing a disease state for which treatment with the compounds described herein results in reduction of the symptoms of the disease. For example, the pathogenic cells can be host cells that are pathogenic under some circumstances such as cells of the immune system that are responsible for graft versus host disease, but not pathogenic under other circumstances.

Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The invention can be utilized to treat such cancers as carcinomas, sarcomas, lymphomas, Hodgekin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

In any of the embodiments described herein heteroatom linkers can be —NR¹R²—, oxygen, sulfur, and the formulae —(NHR¹NHR²)—, —SO—, —(SO₂)—, and —N(R³)O—, wherein R¹, R², and R³ are each independently selected from hydrogen, alkyl, aryl, arylalkyl, substituted aryl, substituted arylalkyl, heteroaryl, substituted heteroaryl, and alkoxyalkyl.

The releasable linkers can be methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio, and carbonylalkylthio, wherein each of the releasable linkers is optionally substituted with a substituent X², as defined below.

In any of the embodiments described herein, the heteroatom linker can be oxygen, and the releasable linkers can be methylene, 1-alkoxyalkylene, 1-alkoxycycloalkylene, 1-alkoxyalkylenecarbonyl, and 1-alkoxycycloalkylenecarbonyl, wherein each of the releasable linkers is optionally substituted with a substituent X², as defined below, and the releasable linker is bonded to the oxygen to form an acetal or ketal. Alternatively, the heteroatom linker can be oxygen, and the releasable linker can be methylene, wherein the methylene is substituted with an optionally-substituted aryl, and the releasable linker is bonded to the oxygen to form an acetal or ketal. Further, the heteroatom linker can be oxygen, and the releasable linker can be sulfonylalkyl, and the releasable linker is bonded to the oxygen to form an alkylsulfonate.

In another embodiment of the above releasable linker embodiment, the heteroatom linker can be nitrogen, and the releasable linkers can be iminoalkylidenyl, carbonylalkylideniminyl, iminocycloalkylidenyl, and carbonylcycloalkylideniminyl, wherein each of the releasable linkers is optionally substituted with a substituent X², as defined below, and the releasable linker is bonded to the nitrogen to form an hydrazone. In an alternate configuration, the hydrazone may be acylated with a carboxylic acid derivative, an orthoformate derivative, or a carbamoyl derivative to form various acylhydrazone releasable linkers.

Alternatively, the heteroatom linker can be oxygen, and the releasable linkers can be alkylene(dialkylsilyl), alkylene(alkylarylsilyl), alkylene(diarylsilyl), (dialkylsilyl)aryl, (alkylarylsilyl)aryl, and (diarylsilyl)aryl, wherein each of the releasable linkers is optionally substituted with a substituent X², as defined below, and the releasable linker is bonded to the oxygen to form a silanol.

In the above releasable linker embodiment, the drug can include a nitrogen atom, the heteroatom linker can be nitrogen, and the releasable linkers can be carbonylarylcarbonyl, carbonyl(carboxyaryl)carbonyl, carbonyl(biscarboxyaryl)carbonyl, and the releasable linker can be bonded to the heteroatom nitrogen to form an amide, and also bonded to the drug nitrogen to form an amide.

In the above releasable linker embodiment, the drug can include an oxygen atom, the heteroatom linker can be nitrogen, and the releasable linkers can be carbonylarylcarbonyl, carbonyl(carboxyaryl)carbonyl, carbonyl(biscarboxyaryl)carbonyl, and the releasable linker can be bonded to the heteroatom linker nitrogen to form an amide, and also bonded to the drug oxygen to form an ester.

The substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides.

In this embodiment the heteroatom linker can be nitrogen, and the substituent X² and the heteroatom linker can be taken together with the releasable linker to which they are bound to form an heterocycle.

The heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

In one aspect of the various conjugates described herein, the polyvalent linker comprises a 3-thiosuccinimid-1-ylalkyloxymethyloxy moiety, where the methyl is optionally substituted with alkyl or substituted aryl.

In another aspect, the polyvalent linker comprises a 3-thiosuccinimid-1-ylalkylcarbonyl, where the carbonyl forms an acylaziridine with the drug, or analog or derivative thereof.

In another aspect, the polyvalent linker comprises a 1-alkoxycycloalkylenoxy moiety.

In another aspect, the polyvalent linker comprises an alkyleneaminocarbonyl(dicarboxylarylene)carboxylate.

In another aspect, the polyvalent linker comprises a dithioalkylcarbonylhydrazide, where the hydrazide forms an hydrazone with the drug, or analog or derivative thereof.

In another aspect, the polyvalent linker comprises a 3-thiosuccinimid-1-ylalkylcarbonylhydrazide, where the hydrazide forms a hydrazone with the drug, or analog or derivative thereof.

In another aspect, the polyvalent linker comprises a 3-thioalkylsulfonylalkyl(disubstituted silyl)oxy, where the disubstituted silyl is substituted with alkyl or optionally substituted aryl.

In another aspect, the polyvalent linker comprises a plurality of spacer linkers selected from the group consisting of the naturally occurring amino acids and stereoisomers thereof.

In another aspect, the polyvalent linker comprises a 2-dithioalkyloxycarbonyl, where the carbonyl forms a carbonate with the drug, or analog or derivative thereof.

In another aspect, the polyvalent linker comprises a 2-dithioarylalkyloxycarbonyl, where the carbonyl forms a carbonate with the drug, or analog or derivative thereof, and the aryl is optionally substituted.

In another aspect, the polyvalent linker comprises a 4-dithioarylalkyloxycarbonyl, where the carbonyl forms a carbonate with the drug, or analog or derivative thereof, and the aryl is optionally substituted.

In another aspect, the polyvalent linker comprises a 3-thiosuccinimid-1-ylalkyloxyalkyloxyalkylidene, where the alkylidene forms an hydrazone with the drug, or analog or derivative thereof, each alkyl is independently selected, and the oxyalkyloxy is optionally substituted with alkyl or optionally substituted aryl.

In another aspect, the polyvalent linker comprises a 2-dithioalkyloxycarbonylhydrazide.

In another aspect, the polyvalent linker comprises a 2- or 3-dithioalkylamino, where the amino forms a vinylogous amide with the drug, or analog or derivative thereof.

In another aspect, the polyvalent linker comprises a 2-dithioalkylamino, where the amino forms a vinylogous amide with the drug, or analog or derivative thereof, and the alkyl is ethyl.

In another aspect, the polyvalent linker comprises a 2- or 3-dithioalkylaminocarbonyl, where the carbonyl forms a carbamate with the drug, or analog or derivative thereof.

In another aspect, the polyvalent linker comprises a releasable linker, a spacer linker, and a releasable linker taken together to form 2-dithioalkylaminocarbonyl, where the carbonyl forms a carbamate with the drug, or analog or derivative thereof, and the alkyl is ethyl.

In another aspect, the polyvalent linker comprises a 2-dithioarylalkyloxycarbonyl, where the carbonyl forms a carbamate or a carbamoylaziridine with the drug, or analog or derivative thereof.

In another aspect, the polyvalent linker comprises a 4-dithioarylalkyloxycarbonyl, where the carbonyl forms a carbamate or a carbamoylaziridine with the drug, or analog or derivative thereof.

In one embodiment, the polyvalent linkers described herein comprise divalent linkers of formulae (II)

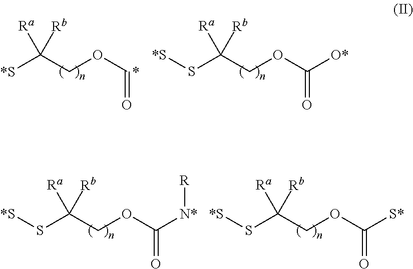

where n is an integer selected from 1 to about 4; R^{a} and R^{b} are each independently selected from the group consisting of hydrogen and alkyl, including lower alkyl such as C₁-C₄ alkyl that are optionally branched; or R^{a} and R^{b} are taken together with the attached carbon atom to form a carbocyclic ring; R is an optionally substituted alkyl group, an optionally substituted acyl group, or a suitably selected nitrogen protecting group; and (*) indicates points of attachment for the drug, vitamin, imaging agent, diagnostic agent, other bivalent linkers, or other parts of the conjugate.

In another embodiment, the polyvalent linkers described herein comprise divalent linkers of formulae (III)

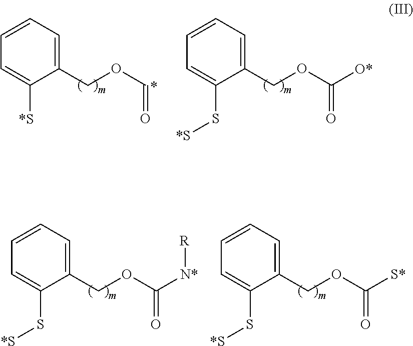

where m is an integer selected from 1 to about 4; R is an optionally substituted alkyl group, an optionally substituted acyl group, or a suitably selected nitrogen protecting group; and (*) indicates points of attachment for the drug, vitamin, imaging agent, diagnostic agent, other bivalent linkers, or other parts of the conjugate.

In another embodiment, the polyvalent linkers described herein comprise divalent linkers of formulae (IV)

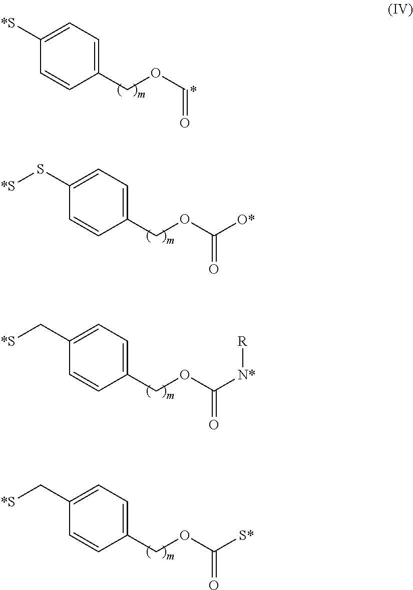

where m is an integer selected from 1 to about 4; R is an optionally substituted alkyl group, an optionally substituted acyl group, or a suitably selected nitrogen protecting group; and (*) indicates points of attachment for the drug, vitamin, imaging agent, diagnostic agent, other divalent linkers, or other parts of the conjugate.

The drug can be any molecule capable of modulating or otherwise modifying cell function, including pharmaceutically active compounds. Suitable molecules can include, but are not limited to: peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins; antigens and antibodies thereto; haptens and antibodies thereto; hormones, lipids, phospholipids, liposomes; toxins; antibiotics; analgesics; bronchodilators; beta-blockers; antimicrobial agents; antihypertensive agents; cardiovascular agents including antiarrhythmics, cardiac glycosides, antianginals and vasodilators; central nervous system agents including stimulants, psychotropics, antimanics, and depressants; antiviral agents; antihistamines; cancer drugs including chemotherapeutic agents; tranquilizers; anti-depressants; H-2 antagonists; anticonvulsants; antinauseants; prostaglandins and prostaglandin analogs; muscle relaxants; anti-inflammatory substances; immunosuppressants, stimulants; decongestants; antiemetics; diuretics; antispasmodics; antiasthmatics; anti-Parkinson agents; expectorants; cough suppressants; mucolytics; and mineral and nutritional additives.

Further, the drug can be any drug known in the art which is cytotoxic, enhances tumor permeability, inhibits tumor cell proliferation, promotes apoptosis, decreases anti-apoptotic activity in target cells, is used to treat diseases caused by infectious agents, enhances an endogenous immune response directed to the pathogenic cells, or is useful for treating a disease state caused by any type of pathogenic cell. Drugs suitable for use in accordance with this invention include adrenocorticoids and cortico steroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, rhizoxin, T2 toxin, plant alkaloids, prednisone, hydroxyurea, teniposide, mitomycins, discodermolides, microtubule inhibitors, epothilones, tubulysins, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vinca alkaloids, such as vincristine, vinblastine, vindesine, vinorelbine and analogs and derivative thereof such as deacetylvinblastine monohydrazide (DAVLBH), colchicine, colchicine derivatives, allocolchicine, thiocolchicine, trityl cysteine, halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin. Other drugs that can be used in accordance with the invention include rapamycins, such as sirolimus or everolimus, penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, and any other art-recognized antimicrobial compound.

In another embodiment, the drug is selected from a cryptophycin, bortezomib, thiobortezomib, a tubulysin, aminopterin, rapamycin, paclitaxel, docetaxel, doxorubicin, daunorubicin, everolimus, α-amanatin, verucarin, didemnin B, geldanomycin, purvalanol A, everolimus, ispinesib, budesonide, dasatinib, an epothilone, a maytansine, and a tyrosine kinase inhibitor, including analogs and derivatives of the foregoing. In another embodiment, the conjugate includes at least two drugs (D) selected illustratively from a vinca alkaloid, a cryptophycin, bortezomib, thiobortezomib, a tubulysin, aminopterin, a rapamycin, such as everolimus or sirolimus, paclitaxel, docetaxel, doxorubicin, daunorubicin, everolimus, α-amanatin, verucarin, didemnin B, geldanomycin, purvalanol A, ispinesib, budesonide, dasatinib, an epothilone, a maytansine, and a tyrosine kinase inhibitor, including analogs and derivatives of the foregoing. In one variation, the drugs (D) are the same. In another variation, the drugs (D) are different.

The drug delivery conjugates described herein can be administered in a combination therapy with any other known drug whether or not the additional drug is targeted. Illustrative additional drugs include, but are not limited to, peptides, oligopeptides, retro-inverso oligopeptides, proteins, protein analogs in which at least one non-peptide linkage replaces a peptide linkage, apoproteins, glycoproteins, enzymes, coenzymes, enzyme inhibitors, amino acids and their derivatives, receptors and other membrane proteins, antigens and antibodies thereto, haptens and antibodies thereto, hormones, lipids, phospholipids, liposomes, toxins, antibiotics, analgesics, bronchodilators, beta-blockers, antimicrobial agents, antihypertensive agents, cardiovascular agents including antiarrhythmics, cardiac glycosides, antianginals, vasodilators, central nervous system agents including stimulants, psychotropics, antimanics, and depressants, antiviral agents, antihistamines, cancer drugs including chemotherapeutic agents, tranquilizers, anti-depressants, H-2 antagonists, anticonvulsants, antinauseants, prostaglandins and prostaglandin analogs, muscle relaxants, anti-inflammatory substances, stimulants, decongestants, antiemetics, diuretics, antispasmodics, antiasthmatics, anti-Parkinson agents, expectorants, cough suppressants, mucolytics, and mineral and nutritional additives.

At least one additional composition comprising a therapeutic factor can be administered to the host in combination or as an adjuvant to the above-detailed methodology, to enhance the drug delivery conjugate-mediated elimination of the population of pathogenic cells, or more than one additional therapeutic factor can be administered. The therapeutic factor can be selected from a compound capable of stimulating an endogenous immune response, a chemotherapeutic agent, or another therapeutic factor capable of complementing the efficacy of the administered drug delivery conjugate. The method of the invention can be performed by administering to the host, in addition to the above-described conjugates, compounds or compositions capable of stimulating an endogenous immune response (e.g. a cytokine) including, but not limited to, cytokines or immune cell growth factors such as interleukins 1-18, stem cell factor, basic FGF, EGF, G-CSF, GM-CSF, FLK-2 ligand, HILDA, MIP-1α, TGF-α, TGF-β, M-CSF, IFN-α, IFN-β, IFN-γ, soluble CD23, LIF, and combinations thereof.

Therapeutically effective combinations of these factors can be used. In one embodiment, for example, therapeutically effective amounts of IL-2, for example, in amounts ranging from about 0.1 MIU/m²/dose/day to about 15 MIU/m²/dose/day in a multiple dose daily regimen, and IFN-α, for example, in amounts ranging from about 0.1 MIU/m²/dose/day to about 7.5 MIU/m²/dose/day in a multiple dose daily regimen, can be used along with the drug delivery conjugates to eliminate, reduce, or neutralize pathogenic cells in a host animal harboring the pathogenic cells (MIU=million international units; m²=approximate body surface area of an average human). In another embodiment IL-12 and IFN-α are used in the above-described therapeutically effective amounts for interleukins and interferons, and in yet another embodiment IL-15 and IFN-α are used in the above described therapeutically effective amounts for interleukins and interferons. In an alternate embodiment IL-2, IFN-α or IFN-γ, and GM-CSF are used in combination in the above described therapeutically effective amounts. The invention also contemplates the use of any other effective combination of cytokines including combinations of other interleukins and interferons and colony stimulating factors.

Chemotherapeutic agents, which are, for example, cytotoxic themselves or can work to enhance tumor permeability, are also suitable for use in the method of the invention in combination with the drug delivery conjugates. Such chemotherapeutic agents include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin. Other drugs that can be used in accordance with the invention include penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, maytansines and analogs and derivatives thereof, gemcitabine, and any other art-recognized antimicrobial compound.

In another embodiment, amino acid refers to beta, gamma, and longer amino acids, such as amino acids of the formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornitine, threonine, and the like.

In another embodiment, a folate ligand intermediate is described having the following formula

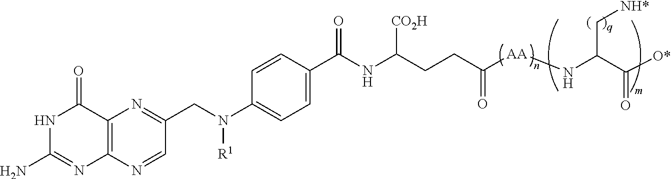

wherein m, n, and q are integers that are independently selected from the range of 0 to about 8; AA is an amino acid, R¹ is hydrogen, alkyl, or a nitrogen protecting group, and drugs are optionally attached at the (*) atoms. In one aspect, AA is a naturally occurring amino acid of either the natural or unnatural configuration. In another aspect, one or more of AA is a hydrophilic amino acid. In another aspect, one or more of AA is Asp and/or Arg. In another aspect, the integer o is 1 or greater. In another aspect, the integer m is 2 or greater. The drugs, or analogs or derivatives thereof, and optionally additional linkers and additional receptor-binding ligands may be connected to the above formula at the free NH side chains of the 2,ω-diaminoalkanoic acid fragments, or at the terminal carboxylate as indicated by the free valences therein.

In another embodiment, a folate ligand intermediate is described having the following formula

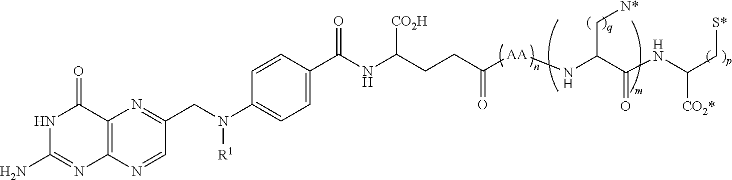

wherein m, n, q, and p are integers that are independently selected from the range of 0 to about 8; AA is an amino acid, R¹ is hydrogen, alkyl, or a nitrogen protecting group, and drugs are optionally attached at the (*) atoms. In one aspect, AA is as a naturally occurring amino acid of either the natural or unnatural configuration. In another aspect, one or more of AA is a hydrophilic amino acid. In another aspect, one or more of AA is Asp and/or Arg. In another aspect, the integers o and p are 1 or greater. In another aspect, the integer m is 2 or greater. The drugs, or analogs or derivatives thereof, and optionally additional linkers and additional receptor-binding ligands may be connected to the above formula at the free NH side chains of the 2,ω-diaminoalkanoic acid fragments, at the cysteinyl thiol groups, or at the terminal carboxylate, as indicated by the free valences therein.

In another embodiment, a folate ligand intermediate is described having the following formula

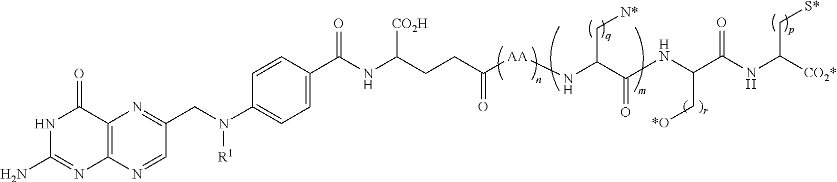

wherein m, n, q, p, and r are integers that are independently selected from the range of 0 to about 8; AA is an amino acid, R¹ is hydrogen, alkyl, or a nitrogen protecting group, and drugs are optionally attached at the (*) atoms. In one aspect, AA is as a naturally occurring amino acid of either the natural or unnatural configuration. In another aspect, one or more of AA is a hydrophilic amino acid. In another aspect, one or more of AA is Asp and/or Arg. In another aspect, the integers o, p, and r are 1 or greater. In another aspect, the integer m is 2 or greater. The drugs, or analogs or derivatives thereof, and optionally additional linkers and additional receptor-binding ligands may be connected to the above formula at the free NH side chains of the 2,ω-diaminoalkanoic acid fragments, at the cyteinyl thiol groups, at the serinyl hydroxy groups, or at the terminal carboxylate, as indicated by the free valences therein.

As used herein, tubulysins refer generally to tetrapeptide compounds of the formula

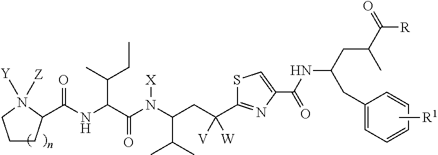

and pharmaceutical salts thereof, where

n is 1-3;

V is H, OR², or halo, and W is H, OR², or alkyl, where R² is independently selected in each instance from H, alkyl, and C(O)R³, where R³ is alkyl, cycloalkyl, alkenyl, aryl, or arylalkyl, each of which is optionally substituted; providing that R² is not H when both V and W are OR²; or V and W are taken together with the attached carbon to form a carbonyl;

X=H, C₁₋₄ alkyl, alkenyl, each of which is optionally substituted, or CH₂QR⁹; where Q is —N—, —O—, or —S—; R⁹=H, C₁₋₄ alkyl, alkenyl, aryl, or C(O)R¹⁰; and R¹⁰=C₁₋₆ alkyl, alkenyl, aryl, or heteroaryl, each of which is optionally substituted;

Z is alkyl and Y is O; or Z is alkyl or C(O)R⁴, and Y is absent, where R⁴ is alkyl, CF₃, or aryl;

R¹ is H, or R¹ represents 1 to 3 substituents selected from halo, nitro, carboxylate or a derivative thereof, cyano, hydroxyl, alkyl, haloalkyl, alkoxy, haloalkoxy, and OR⁶, where R⁶ is hydrogen or optionally substituted aryl, a phenol protecting group, a prodrug moiety, alkyl, arylalkyl, C(O)R⁷, P(O)(OR⁸)₂, or SO₃R⁸, where R⁷ and R⁸ are independently selected in each instance from H, alkyl, alkenyl, cycloalkyl, heterocyclyl, aryl, and arylalkyl, each of which is optionally substituted, or R⁸ is a metal cation; and

R is OH or a leaving group, or R forms a carboxylic acid derivative, such as an acylhydrazide.

Conjugates of each of the foregoing tubulysins are described herein. In one variation, Z is methyl. In another variation, R¹ is H. In another variation, R¹ is OR⁶ at C(4), where R⁶ is H, alkyl, or COR⁷. In another variation, V is H, and W is OC(O)R³. In another variation, X=CH₂QR⁹. In another variation, X=CH₂OR⁹. In another variation, R⁹ is alkyl or alkenyl. In another variation, R⁹ is C(O)R¹⁰. In another variation, R¹⁰=optionally substituted C₁₋₆ alkyl. In another variation, R¹⁰=C₁₋₆ alkyl. In another variation, R forms an acylhydrazide. It is to be understood that the foregoing description is an explicit description of each chemically possible combination of variations of the general tubulysin structure. For example, it is to be understood that the foregoing description is a description of the variation where Z is methyl, and R¹ is H; where R¹ is OR⁶ at C(4), and R⁶ is H; where Z is methyl, R¹ is OR⁶ at C(4), R⁶ is H, and X=CH₂OR⁹; and the like.

Natural tubulysins are generally linear tetrapeptides consisting of N-methyl pipecolic acid (Mep), isoleucine (Ile), an unnatural aminoacid called tubuvalin (Tuv), and either an unnatural aminoacid called tubutyrosine (Tut, an analog of tyrosine) or an unnatural aminoacid called tubuphenylalanine (Tup, an analog of phenylalanine). In another embodiment, naturally occurring tubulysins, and analogs and derivatives thereof, of the following general formula are described

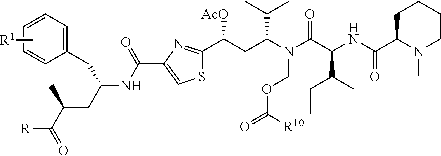

and pharmaceutical salts thereof, where R, R¹, and R¹⁰ are as described in the various embodiments herein. Conjugates of each of the foregoing tubulysins are described herein.

In another embodiment, conjugates of naturally occurring tubulysins of the following general formula are described

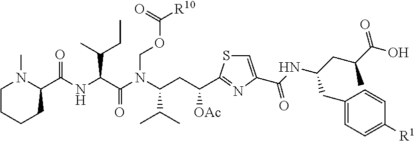

| Factor | R¹⁰ | R¹ |
|---|---|---|
| A | (CH₃)₂CHCH₂ | OH |
| B | CH₃(CH₂)₂ | OH |
| C | CH₃CH₂ | OH |
| D | (CH₃)₂CHCH₂ | H |
| E | CH₃(CH₂)₂ | H |
| F | CH₂CH₃ | H |
| G | (CH₃)₂C═CH | OH |
| H | CH₃ | H |
| I | CH₃ | OH |

and pharmaceutical salts thereof.

In another embodiment, compounds are described herein where the conjugate is formed at the terminal carboxylic acid group or the terminal acylhydrazine group of each of the tybulysins described herein.

It is appreciated that the arrangement and/or orientation of the various hydrophilic linkers may be in a linear or branched fashion, or both. For example, the hydrophilic linkers may form the backbone of the linker forming the conjugate between the folate and the drug, imagining agent, or diagnostic agent. Alternatively, the hydrophilic portion of the linker may be pendant to or attached to the backbone of the chain of atoms connecting the binding ligand B to the drug D. In this latter arrangement, the hydrophilic portion may be proximal or distal to the backbone chain of atoms.

In another embodiment, the linker is more or less linear, and the hydrophilic groups are arranged largely in a series to form a chain-like linker in the conjugate. Said another way, the hydrophilic groups form some or all of the backbone of the linker in this linear embodiment.

In another embodiment, the linker is branched with hydrophilic groups. In this branched embodiment, the hydrophilic groups may be proximal to the backbone or distal to the backbone. In each of these arrangements, the linker is more spherical or cylindrical in shape. In one variation, the linker is shaped like a bottle-brush. In one aspect, the backbone of the linker is formed by a linear series of amides, and the hydrophilic portion of the linker is formed by a parallel arrangement of branching side chains, such as by connecting monosaccharides, sulfonates, and the like, and derivatives and analogs thereof.

It is understood that the linker may be neutral or ionizable under certain conditions, such as physiological conditions encountered in vivo. For ionizable linkers, under the selected conditions, the linker may deprotonate to form a negative ion, or alternatively become protonated to form a positive ion. It is appreciated that more than one deprotonation or protonation event may occur. In addition, it is understood that the same linker may deprotonate and protonate to form inner salts or zwitterionic compounds.

In another embodiment, the hydrophilic spacer linkers are neutral, i.e. under physiological conditions, the linkers do not significantly protonate nor deprotonate. In another embodiment, the hydrophilic spacer linkers may be protonated to carry one or more positive charges. It is understood that the protonation capability is condition dependent. In one aspect, the conditions are physiological conditions, and the linker is protonated in vivo. In another embodiment, the spacers include both regions that are neutral and regions that may be protonated to carry one or more positive charges. In another embodiment, the spacers include both regions that may be deprotonated to carry one or more negative charges and regions that may be protonated to carry one or more positive charges. It is understood that in this latter embodiment that zwitterions or inner salts may be formed.

In one aspect, the regions of the linkers that may be deprotonated to carry a negative charge include carboxylic acids, such as aspartic acid, glutamic acid, and longer chain carboxylic acid groups, and sulfuric acid esters, such as alkyl esters of sulfuric acid. In another aspect, the regions of the linkers that may be protonated to carry a positive charge include amino groups, such as polyaminoalkylenes including ethylene diamines, propylene diamines, butylene diamines and the like, and/or heterocycles including pyrollidines, piperidines, piperazines, and other amino groups, each of which is optionally substituted. In another embodiment, the regions of the linkers that are neutral include poly hydroxyl groups, such as sugars, carbohydrates, saccharides, inositols, and the like, and/or polyether groups, such as polyoxyalkylene groups including polyoxyethylene, polyoxypropylene, and the like.

In one embodiment, the hydrophilic spacer linkers described herein include are formed primarily from carbon, hydrogen, and oxygen, and have a carbon/oxygen ratio of about 3:1 or less, or of about 2:1 or less. In one aspect, the hydrophilic linkers described herein include a plurality of ether functional groups. In another aspect, the hydrophilic linkers described herein include a plurality of hydroxyl functional groups. Illustrative fragments that may be used to form such linkers include polyhydroxyl compounds such as carbohydrates, polyether compounds such as polyethylene glycol units, and acid groups such as carboxyl and alkyl sulfuric acids. In one variation, oligoamide spacers, and the like may also be included in the linker.

Illustrative carbohydrate spacers include saccharopeptides as described herein that include both a peptide feature and sugar feature; glucuronides, which may be incorporated via [2+3] Huisgen cyclization, also known as click chemistry; β-alkyl glycosides, such as of 2-deoxyhexapyranoses (2-deoxyglucose, 2-deoxyglucuronide, and the like), and β-alkyl mannopyranosides. Illustrative PEG groups include those of a specific length range from about 4 to about 20 PEG groups. Illustrative alkyl sulfuric acid esters may also be introduced with click chemistry directly into the backbone. Illustrative oligoamide spacers include EDTA and DTPA spacers, β-amino acids, and the like.

In another embodiment, the hydrophilic spacer linkers described herein include a polyether, such as the linkers of the following formulae:

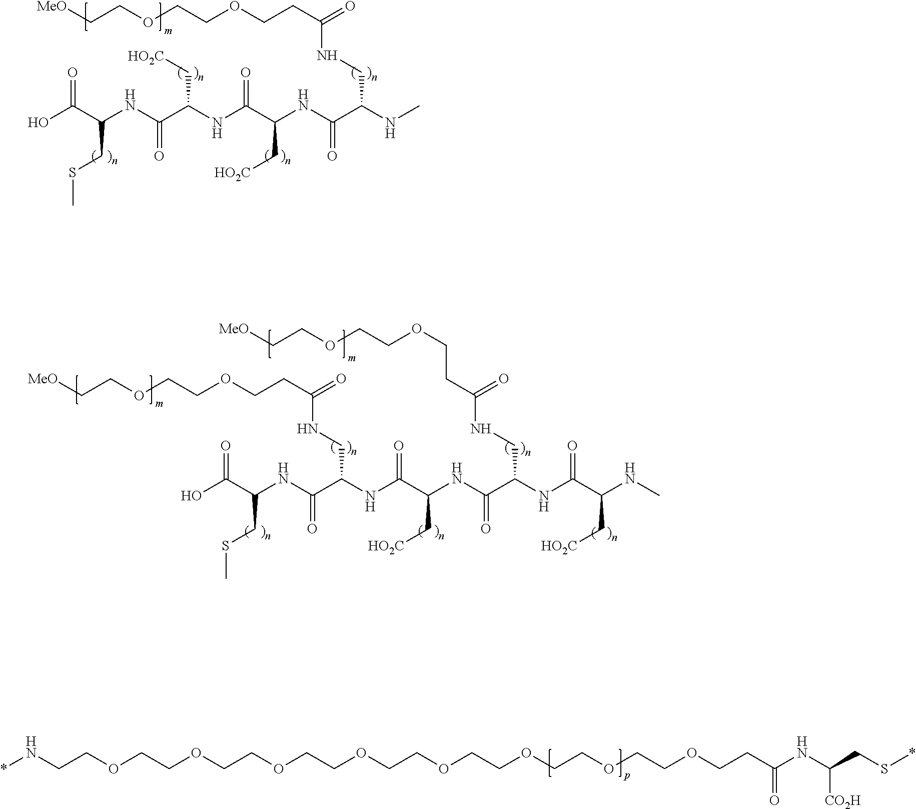

where m is an integer independently selected in each instance from 1 to about 8; p is an integer selected 1 to about 10; and n is an integer independently selected in each instance from 1 to about 3. In one aspect, m is independently in each instance 1 to about 3. In another aspect, n is 1 in each instance. In another aspect, p is independently in each instance about 4 to about 6. Illustratively, the corresponding polypropylene polyethers corresponding to the foregoing are contemplated herein and may be included in the conjugates as hydrophilic spacer linkers. In addition, it is appreciated that mixed polyethylene and polypropylene polyethers may be included in the conjugates as hydrophilic spacer linkers. Further, cyclic variations of the foregoing polyether compounds, such as those that include tetrahydrofuranyl, 1,3-dioxanes, 1,4-dioxanes, and the like are contemplated herein.

In another illustrative embodiment, the hydrophilic spacer linkers described herein include a plurality of hydroxyl functional groups, such as linkers that incorporate monosaccharides, oligosaccharides, polysaccharides, and the like. It is to be understood that the polyhydroxyl containing spacer linkers comprises a plurality of —(CROH)— groups, where R is hydrogen or alkyl.

In another embodiment, the spacer linkers include one or more of the following fragments:

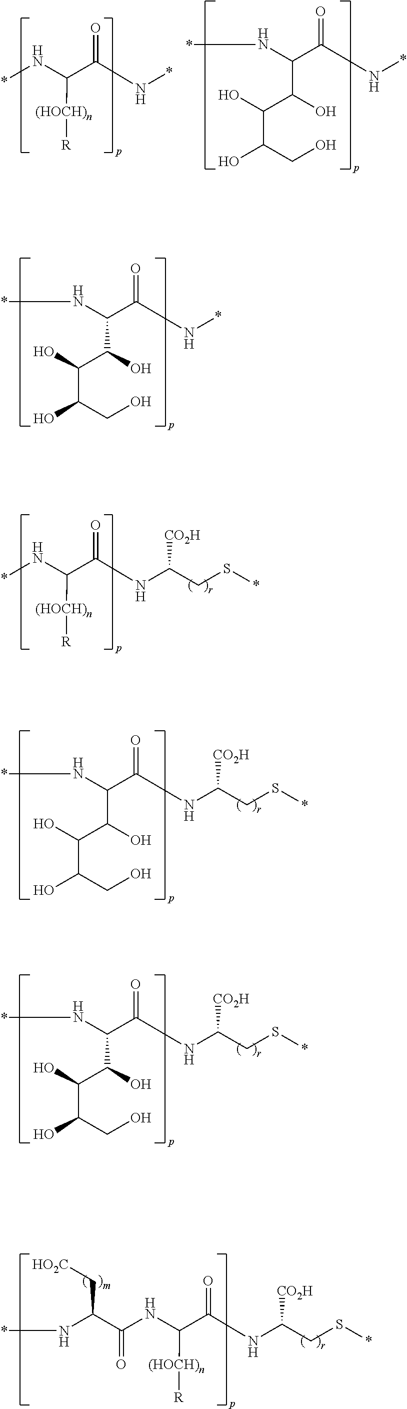

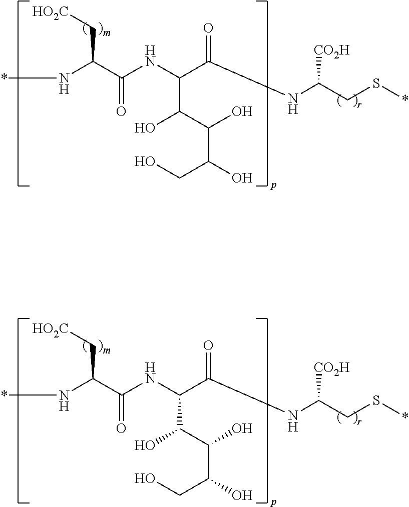

wherein R is H, alkyl, cycloalkyl, or arylalkyl; m is an integer from 1 to about 3; n is an integer from 1 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the spacer linker includes one or more of the following cyclic polyhydroxyl groups:

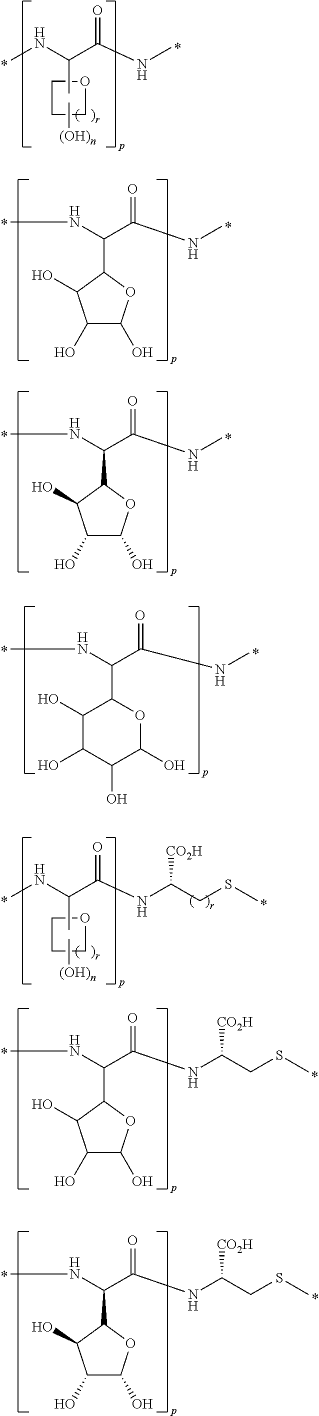

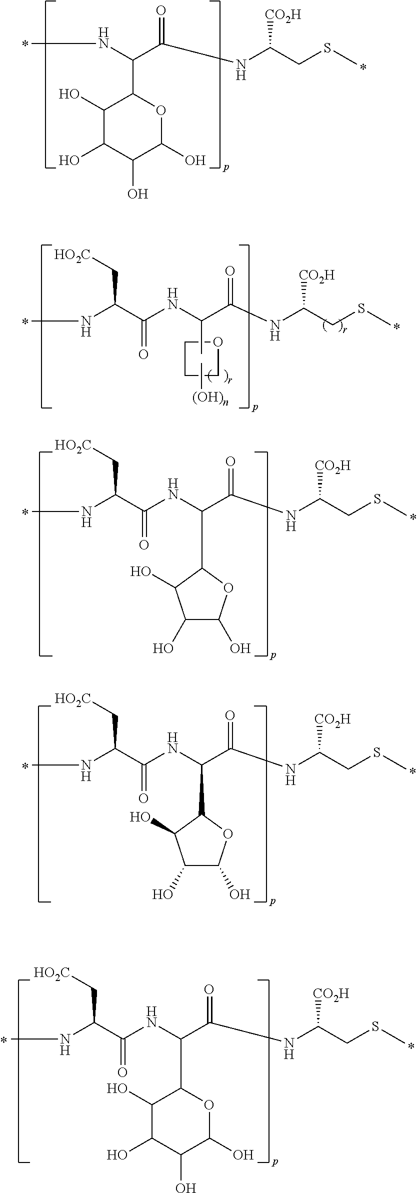

wherein n is an integer from 2 to about 5, p is an integer from 1 to about 5, and r is an integer from 1 to about 4. In one aspect, the integer n is 3 or 4. In another aspect, the integer p is 3 or 4. In another aspect, the integer r is 2 or 3. It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy compounds are also contemplated. Illustratively, compounds of the following formulae are contemplated:

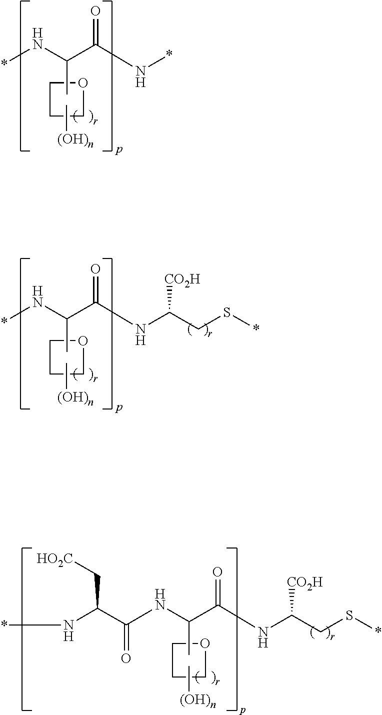

wherein n is equal to or less than r, such as when r is 2 or 3, n is 1 or 2, or 1, 2, or 3, respectively.

In another embodiment, the spacer linker includes a polyhydroxyl compound of the following formula:

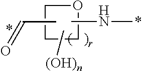

wherein n and r are each an integer selected from 1 to about 3. In one aspect, the spacer linker includes one or more polyhydroxyl compounds of the following formulae:

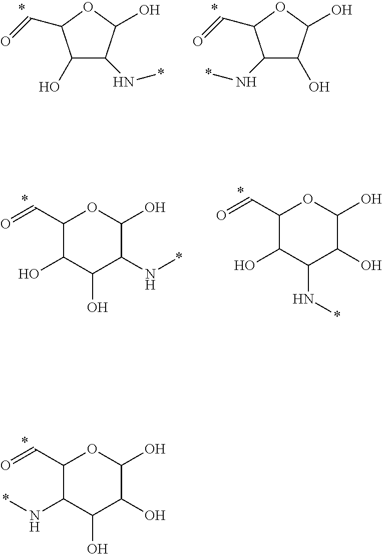 [SMILES: *NC1C(O)OC(C=O)C1O ; *NC1C(C=O)OC(O)C1O ; *NC1C(O)OC(C=O)C(O)C1O ; *NC1C(O)C(O)OC(C=O)C1O ; *NC1C(C=O)OC(O)C(O)C1O]

It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules.

In another configuration, the hydrophilic linkers L described herein include polyhydroxyl groups that are spaced away from the backbone of the linker. In one embodiment, such carbohydrate groups or polyhydroxyl groups are connected to the back bone by a triazole group, forming triazole-linked hydrophilic spacer linkers. Illustratively, such linkers include fragments of the following formulae:

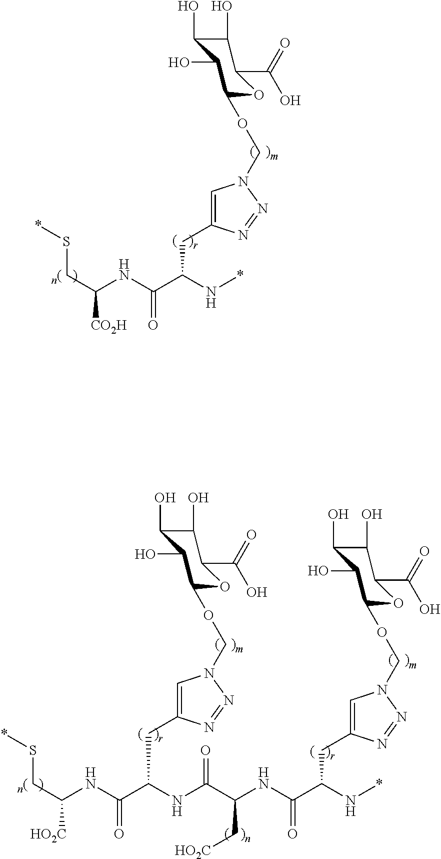

wherein n, m, and r are integers and are each independently selected in each instance from 1 to about 5. In one illustrative aspect, m is independently 2 or 3 in each instance. In another aspect, r is 1 in each instance. In another aspect, n is 1 in each instance. In one variation, the group connecting the polyhydroxyl group to the backbone of the linker is a different heteroaryl group, including but not limited to, pyrrole, pyrazole, 1,2,4-triazole, furan, oxazole, isoxazole, thienyl, thiazole, isothiazole, oxadiazole, and the like. Similarly, divalent 6-membered ring heteroaryl groups are contemplated. Other variations of the foregoing illustrative hydrophilic spacer linkers include oxyalkylene groups, such as the following formulae:

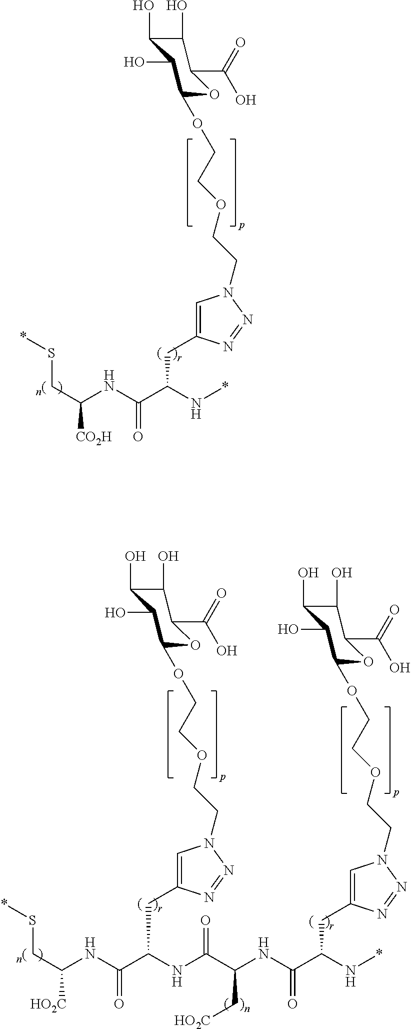

wherein n and r are integers and are each independently selected in each instance from 1 to about 5; and p is an integer selected from 1 to about 4.

In another embodiment, such carbohydrate groups or polyhydroxyl groups are connected to the back bone by an amide group, forming amide-linked hydrophilic spacer linkers. Illustratively, such linkers include fragments of the following formulae:

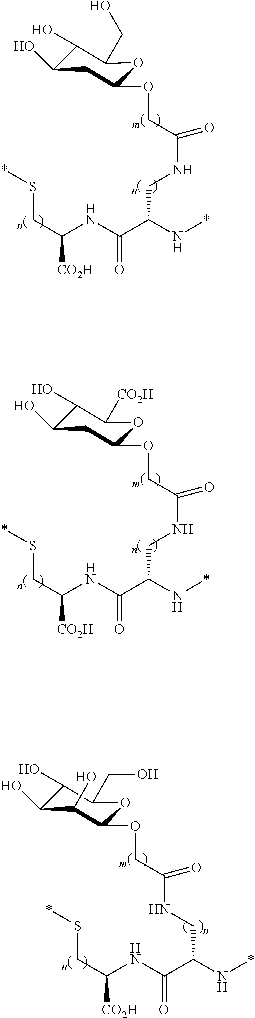

wherein n is an integer selected from 1 to about 3, and m is an integer selected from 1 to about 22. In one illustrative aspect, n is 1 or 2. In another illustrative aspect, m is selected from about 6 to about 10, illustratively 8. In one variation, the group connecting the polyhydroxyl group to the backbone of the linker is a different functional group, including but not limited to, esters, ureas, carbamates, acylhydrazones, and the like. Similarly, cyclic variations are contemplated. Other variations of the foregoing illustrative hydrophilic spacer linkers include oxyalkylene groups, such as the following formulae:

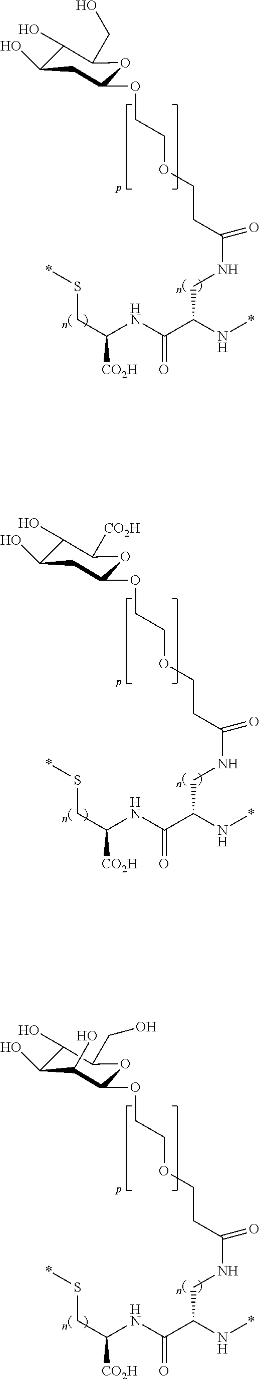

wherein n and r are integers and are each independently selected in each instance from 1 to about 5; and p is an integer selected from 1 to about 4.

In another embodiment, the spacer linkers include one or more of the following fragments:

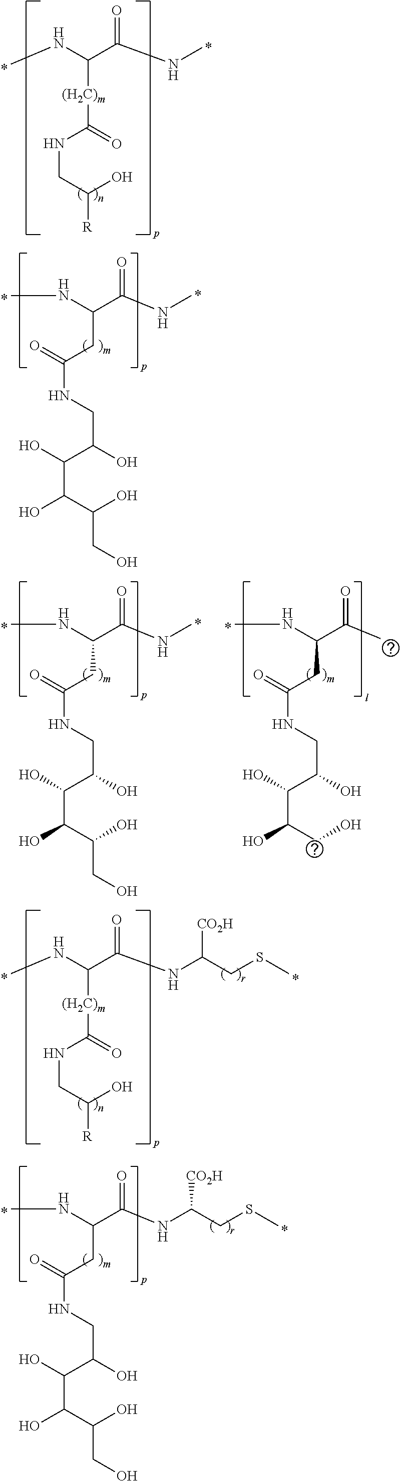

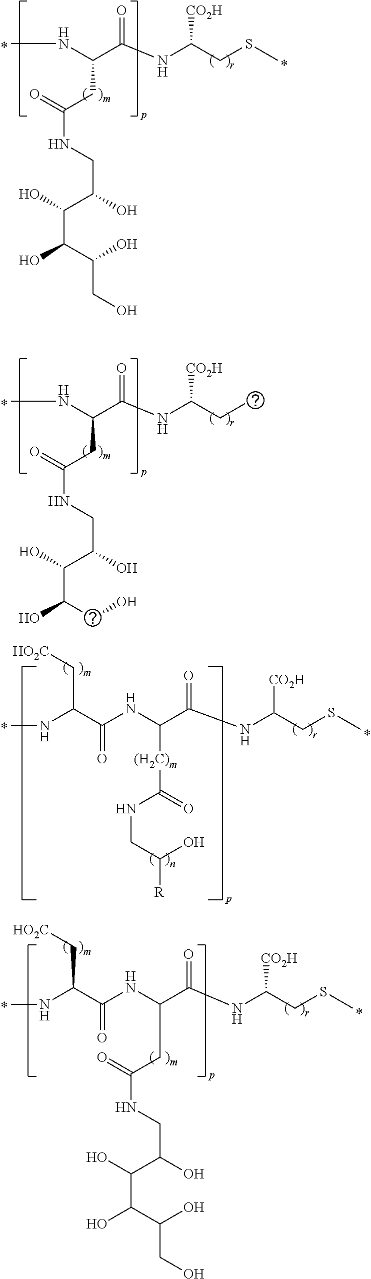

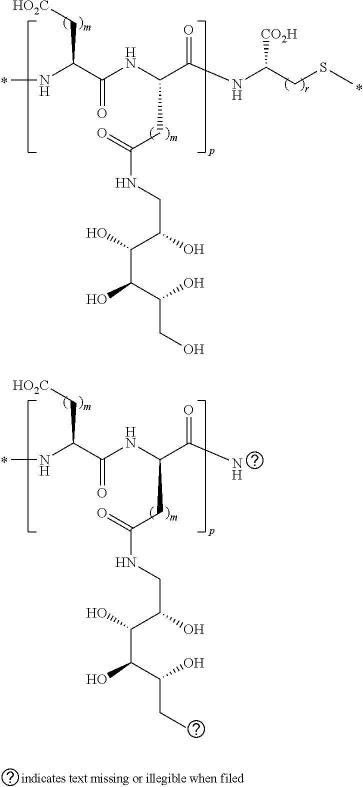

wherein R is H, alkyl, cycloalkyl, or arylalkyl; m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the spacer linkers include one or more of the following fragments:

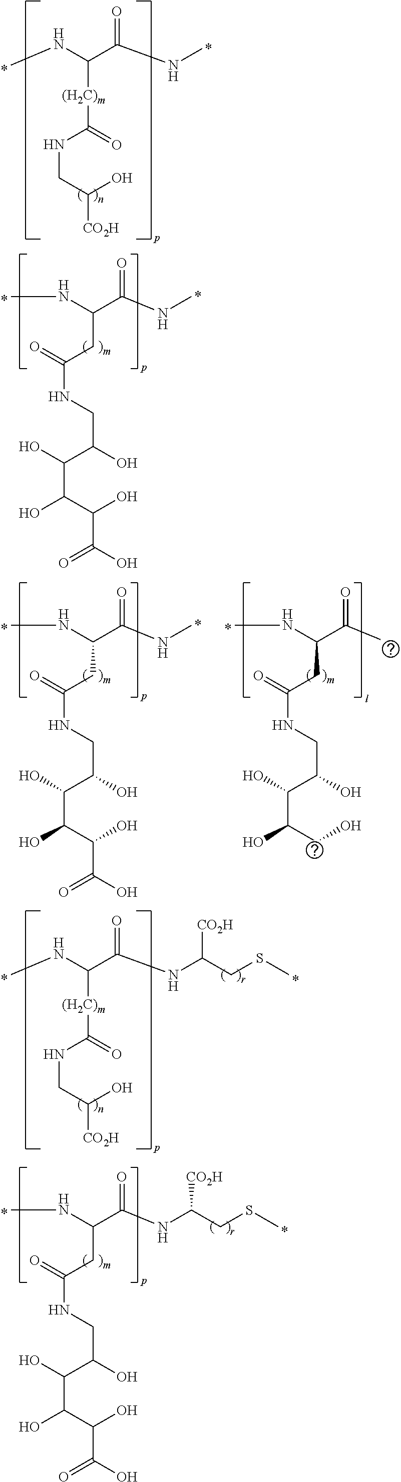

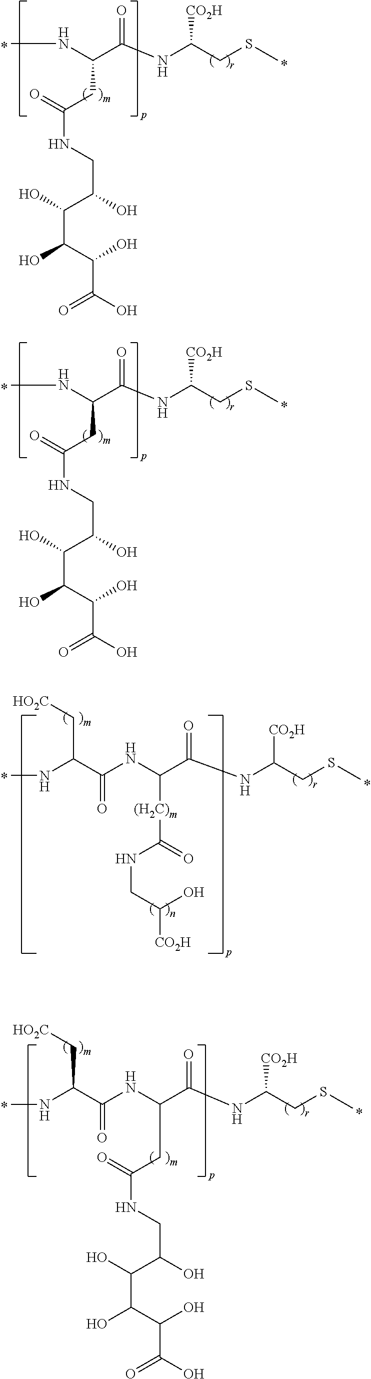

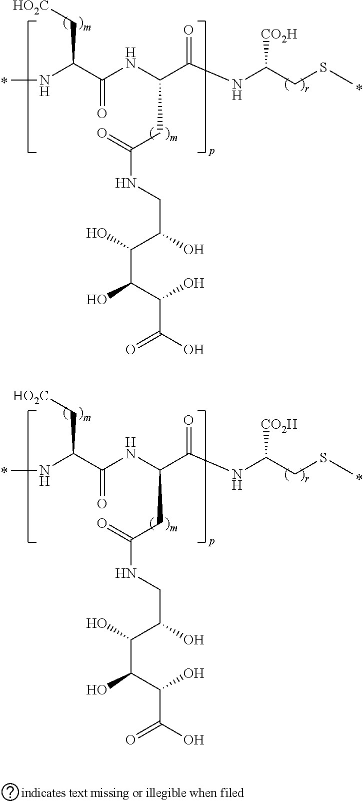

wherein m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the spacer linkers include one or more of the following fragments:

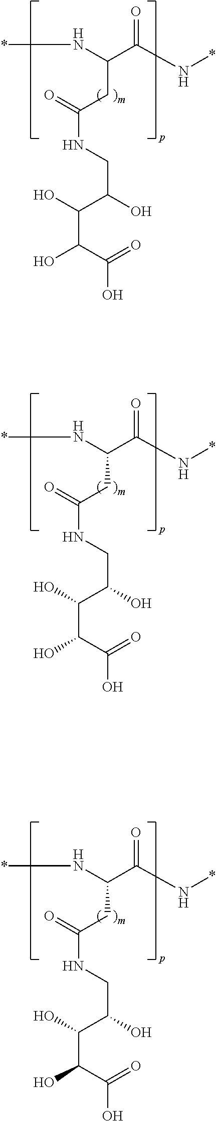

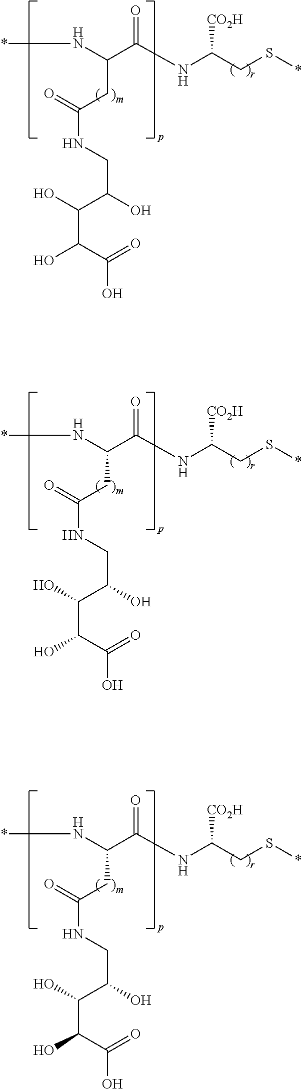

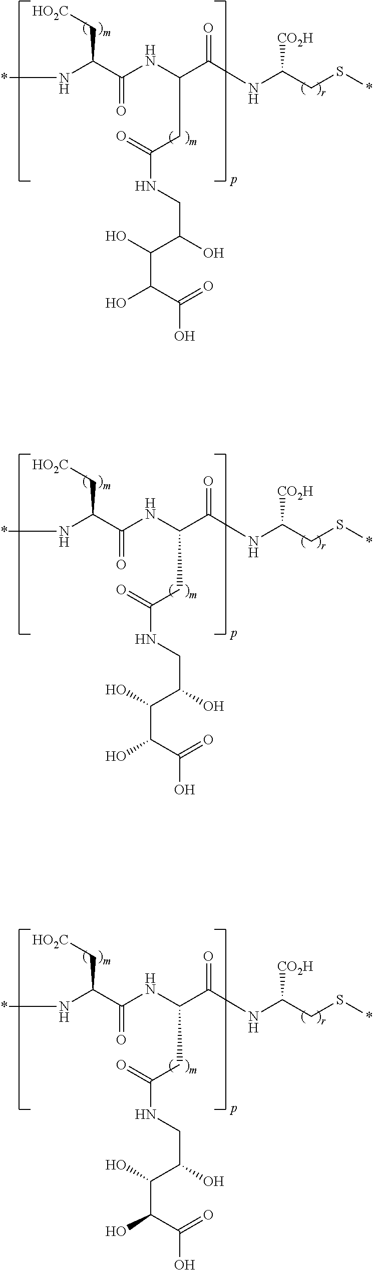

wherein m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the hydrophilic spacer linker is a combination of backbone and branching side motifs such as is illustrated by the following formulae

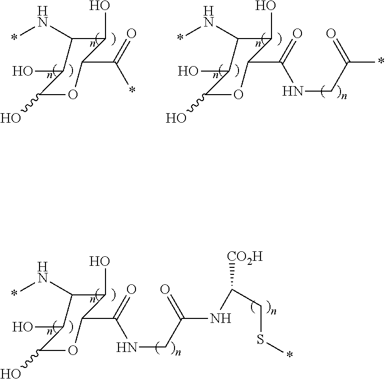

wherein n is an integer independently selected in each instance from 0 to about 3. The above formula are intended to represent 4, 5, 6, and even larger membered cyclic sugars. In addition, it is to be understood that the above formula may be modified to represent deoxy sugars, where one or more of the hydroxy groups present on the formulae are replaced by hydrogen, alkyl, or amino. In addition, it is to be understood that the corresponding carbonyl compounds are contemplated by the above formulae, where one or more of the hydroxyl groups is oxidized to the corresponding carbonyl. In addition, in this illustrative embodiment, the pyranose includes both carboxyl and amino functional groups and (a) can be inserted into the backbone and (b) can provide synthetic handles for branching side chains in variations of this embodiment. Any of the pendant hydroxyl groups may be used to attach other chemical fragments, including additional sugars to prepare the corresponding oligosaccharides. Other variations of this embodiment are also contemplated, including inserting the pyranose or other sugar into the backbone at a single carbon, i.e. a spiro arrangement, at a geminal pair of carbons, and like arrangements. For example, one or two ends of the linker, or the drug D, or the binding ligand B may be connected to the sugar to be inserted into the backbone in a 1,1; 1,2; 1,3; 1,4; 2,3, or other arrangement.

In another embodiment, the hydrophilic spacer linkers described herein include are formed primarily from carbon, hydrogen, and nitrogen, and have a carbon/nitrogen ratio of about 3:1 or less, or of about 2:1 or less. In one aspect, the hydrophilic linkers described herein include a plurality of amino functional groups.

In another embodiment, the spacer linkers include one or more amino groups of the following formulae:

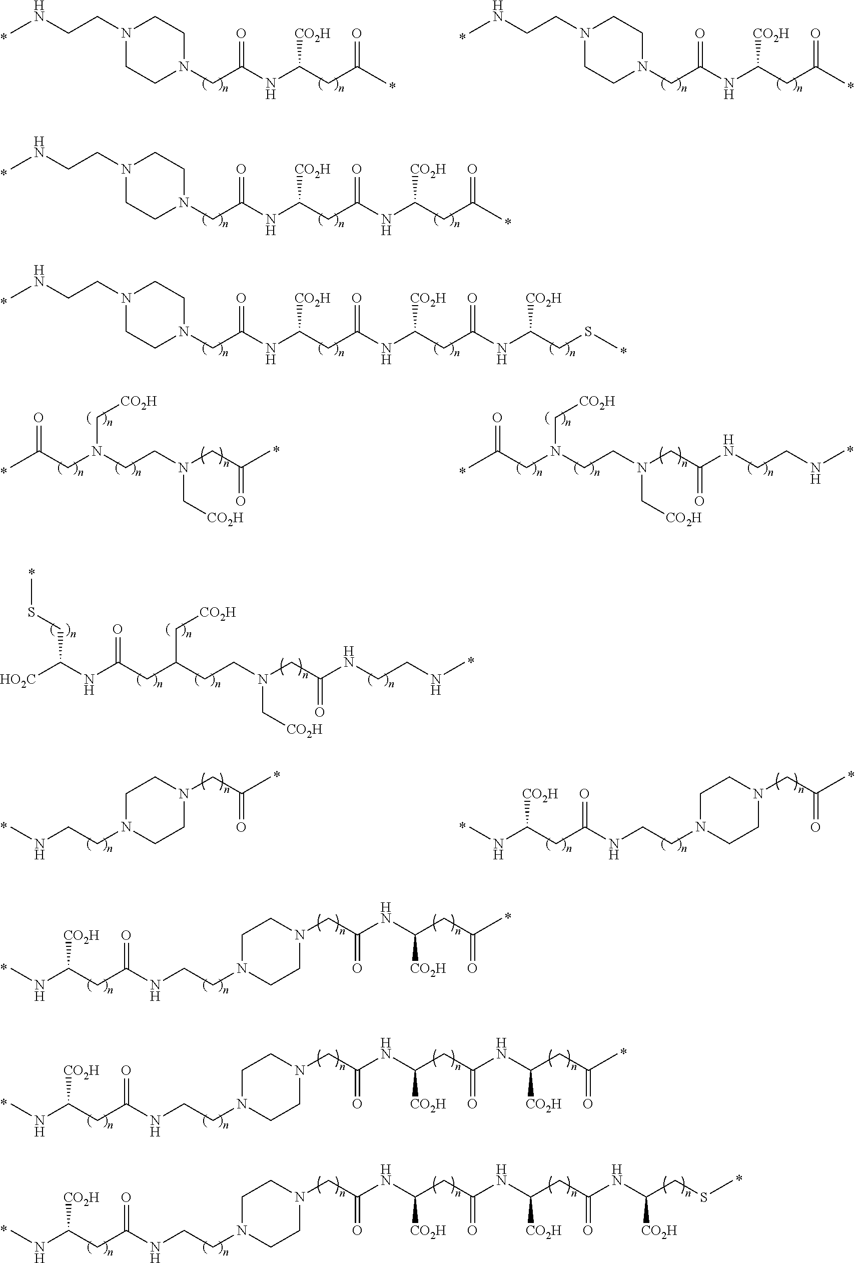

where n is an integer independently selected in each instance from 1 to about 3. In one aspect, the integer n is independently 1 or 2 in each instance. In another aspect, the integer n is 1 in each instance.

In another embodiment, the hydrophilic spacer linker is a sulfuric acid ester, such as an alkyl ester of sulfuric acid. Illustratively, the spacer linker is of the following formula:

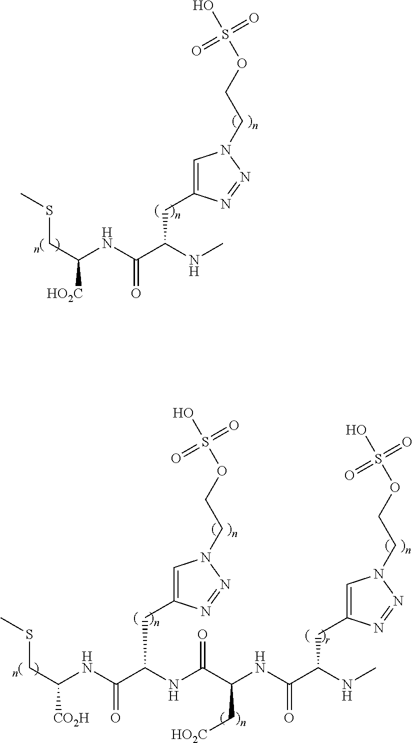

where n is an integer independently selected in each instance from 1 to about 3. Illustratively, n is independently 1 or 2 in each instance.

It is understood, that in such polyhydroxyl, polyamino, carboxylic acid, sulfuric acid, and like linkers that include free hydrogens bound to heteroatoms, one or more of those free hydrogen atoms may be protected with the appropriate hydroxyl, amino, or acid protecting group, respectively, or alternatively may be blocked as the corresponding pro-drugs, the latter of which are selected for the particular use, such as pro-drugs that release the parent drug under general or specific physiological conditions.

In each of the foregoing illustrative examples of linkers L, there are also included in some cases additional spacer linkers L_{S}, and/or additional releasable linkers L_{R}. Those spacer linker and releasable linkers also may include asymmetric carbon atoms. It is to be further understood that the stereochemical configurations shown herein are merely illustrative, and other stereochemical configurations are contemplated. For example in one variation, the corresponding unnatural amino acid configurations may be included in the conjugated described herein as follows:

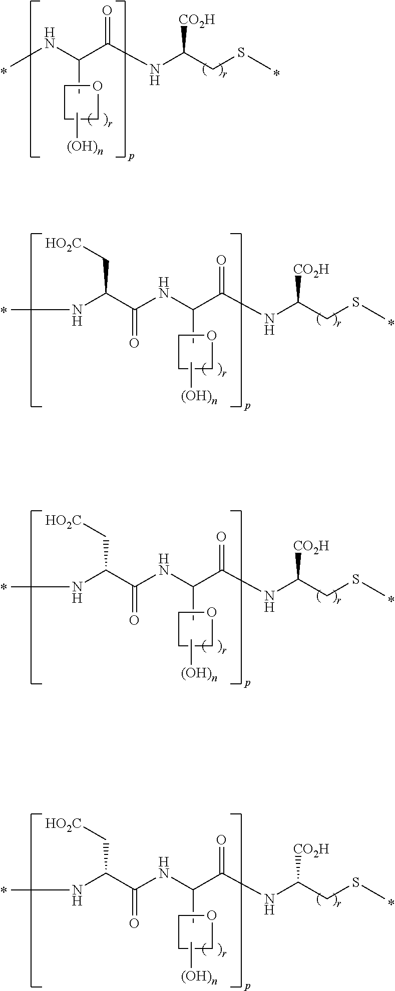

wherein n is an integer from 2 to about 5, p is an integer from 1 to about 5, and r is an integer from 1 to about 4, as described above.

It is to be further understood that in the foregoing embodiments, open positions, such as (*) atoms are locations for attachment of the binding ligand (B) or the drug (D) to be delivered. In addition, it is to be understood that such attachment of either or both of B and A may be direct or through an intervening linker. Intervening linkers include other spacer linkers and/or releasable linkers. Illustrative additional spacer linkers and releasable linkers that are included in the conjugated described herein are described in U.S. patent application Ser. No. 10/765,335, the disclosure of which is incorporated herein by reference.

In one embodiment, the hydrophilic spacer linker comprises one or more carbohydrate containing or polyhydroxyl group containing linkers. In another embodiment, the hydrophilic spacer linker comprises at least three carbohydrate containing or polyhydroxyl group containing linkers. In another embodiment, the hydrophilic spacer linker comprises one or more carbohydrate containing or polyhydroxyl group containing linkers, and one or more aspartic acids. In another embodiment, the hydrophilic spacer linker comprises one or more carbohydrate containing or polyhydroxyl group containing linkers, and one or more glutamic acids. In another embodiment, the hydrophilic spacer linker comprises one or more carbohydrate containing or polyhydroxyl group containing linkers, one or more glutamic acids, one or more aspartic acids, and one or more beta amino alanines. In a series of variations, in each of the foregoing embodiments, the hydrophilic spacer linker also includes one or more cysteines. In another series of variations, in each of the foregoing embodiments, the hydrophilic spacer linker also includes at least one arginine.

In another embodiment, the hydrophilic spacer linker comprises one or more divalent 1,4-piperazines that are included in the chain of atoms connecting at least one of the binding ligands (L) with at least one of the drugs (D). In one variation, the hydrophilic spacer linker includes one or more carbohydrate containing or polyhydroxyl group containing linkers.

In another variation, the hydrophilic spacer linker includes one or more carbohydrate containing or polyhydroxyl group containing linkers and one or more aspartic acids. In another variation, the hydrophilic spacer linker includes one or more carbohydrate containing or polyhydroxyl group containing linkers and one or more glutamic acids. In a series of variations, in each of the foregoing embodiments, the hydrophilic spacer linker also includes one or more cysteines. In another series of variations, in each of the foregoing embodiments, the hydrophilic spacer linker also includes at least one arginine.

In another embodiment, the hydrophilic spacer linker comprises one or more oligoamide hydrophilic spacers, such as but not limited to aminoethylpiperazinylacetamide.

In another embodiment, the hydrophilic spacer linker comprises one or more triazole linked carbohydrate containing or polyhydroxyl group containing linkers. In another embodiment, the hydrophilic spacer linker comprises one or more amide linked carbohydrate containing or polyhydroxyl group containing linkers. In another embodiment, the hydrophilic spacer linker comprises one or more PEG groups and one or more cysteines. In another embodiment, the hydrophilic spacer linker comprises one or more EDTE derivatives.

Illustrative embodiments of vitamin analogs and/or derivatives include folate and analogs and derivatives of folate such as folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this invention are the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are conventionally termed “folates,” reflecting their ability to bind with folate-receptors, and such ligands when conjugated with exogenous molecules are effective to enhance transmembrane transport, such as via folate-mediated endocytosis as described herein.

In another embodiment, the drug has the formula

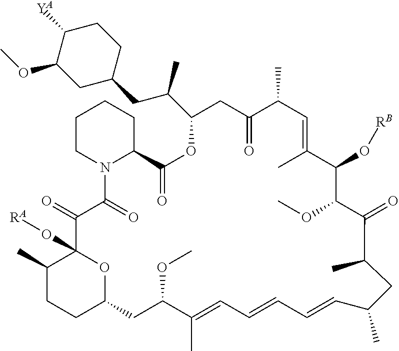

wherein

Y^{A} is OR^{C} or OCH₂CH₂OR^{C};

one of R^{A}, R^{B}, or R^{C} is a bond connected to L; and

the other two of R^{A}, R^{B}, and R^{C} are independently selected in each case from the group consisting of hydrogen, optionally substituted heteroalkyl, prodrug forming group, and C(O)R^{D}, where R^{D} is in each instance independently selected from the group consisting of hydrogen, and alkyl, alkenyl, heteroalkyl, cycloalkyl, cycloheteroalkyl, aryl, arylalkyl, heteroaryl, and heteroarylalkyl, each of which is optionally substituted is described.

In another embodiment, the compound of any of the embodiment described herein wherein L comprises a divalent linker of the formula

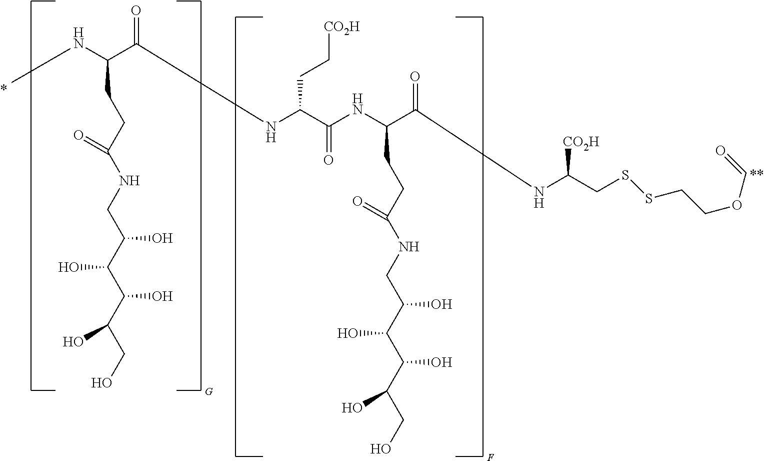

wherein * indicates the point of attachment to a folate and ** indicates the point of attachment to a drug; and F and G are each independently 1, 2, 3 or 4 are described.

In another embodiment, the of any one of the embodiments described herein wherein L is a linker comprises a divalent linker of the formula

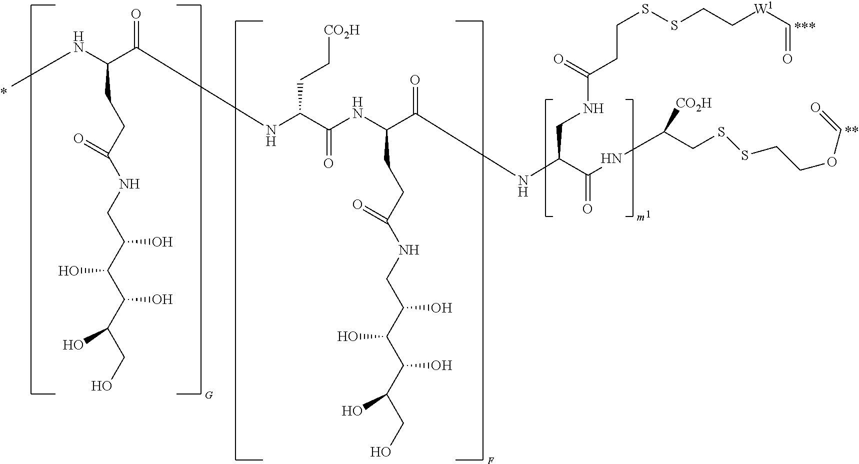

wherein *, **, *** each indicate points of attachment to the folate receptor binding moiety B, and the one or more drugs D. It is to be understood that when there are fewer drugs, *, **, *** are substituted with hydrogen or a heteroatom. F and G are each independently 1, 2, 3 or 4; and W¹ is NH or O is described. In another aspect, m¹ is 0 or 1.

In another embodiment, the method or pharmaceutical composition of any one of the preceding embodiments wherein the disease is selected from the group consisting of arthritis, including rheumatoid arthritis and osteoarthritis, glomerulonephritis, proliferative retinopathy, restenosis, ulcerative colitis, Crohn's disease, fibromyalgia, psoriasis and other inflammations of the skin, osteomyelitis, Sjögren's syndrome, multiple sclerosis, diabetes, atherosclerosis, pulmonary fibrosis, lupus erythematosus, sarcoidosis, systemic sclerosis, organ transplant rejection (GVHD) and chronic inflammations is described.

The compounds described herein may contain one or more chiral centers, or may otherwise be capable of existing as multiple stereoisomers. It is to be understood that in one embodiment, the invention described herein is not limited to any particular sterochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may be include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. Illustratively, such particularly limited length alkyl groups, including C₁-C₈, C₁-C₆, and C₁-C₄ may be referred to as lower alkyl. It is to be further understood that in certain embodiments alkenyl and/or alkynyl may each be advantageously of limited length, including C₂-C₂₄, C₂-C₁₂, C₂-C₈, C₂-C₆, and C₂-C₄. Illustratively, such particularly limited length alkenyl and/or alkynyl groups, including C₂-C₈, C₂-C₆, and C₂-C₄ may be referred to as lower alkenyl and/or alkynyl. It is appreciated herein that shorter alkyl, alkenyl, and/or alkynyl groups may add less lipophilicity to the compound and accordingly will have different pharmacokinetic behavior. In embodiments of the invention described herein, it is to be understood, in each case, that the recitation of alkyl refers to alkyl as defined herein, and optionally lower alkyl. In embodiments of the invention described herein, it is to be understood, in each case, that the recitation of alkenyl refers to alkenyl as defined herein, and optionally lower alkenyl. In embodiments of the invention described herein, it is to be understood, in each case, that the recitation of alkynyl refers to alkynyl as defined herein, and optionally lower alkynyl. Illustrative alkyl, alkenyl, and alkynyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl, and the like, and the corresponding groups containing one or more double and/or triple bonds, or a combination thereof.

As used herein, the term “alkylene” includes a divalent chain of carbon atoms, which is optionally branched. As used herein, the term “alkenylene” and “alkynylene” includes a divalent chain of carbon atoms, which is optionally branched, and includes at least one double bond or triple bond, respectively. It is to be understood that alkynylene may also include one or more double bonds. It is to be further understood that in certain embodiments, alkylene is advantageously of limited length, including C₁-C₂₄, C₁-C₁₂, C₁-C₈, C₁-C₆, and C₁-C₄. Illustratively, such particularly limited length alkylene groups, including C₁-C₈, C₁-C₆, and C₁-C₄ may be referred to as lower alkylene. It is to be further understood that in certain embodiments alkenylene and/or alkynylene may each be advantageously of limited length, including C₂-C₂₄, C₂-C₁₂, C₂-C₈, C₂-C₆, and C₂-C₄. Illustratively, such particularly limited length alkenylene and/or alkynylene groups, including C₂-C₈, C₂-C₆, and C₂-C₄ may be referred to as lower alkenylene and/or alkynylene. It is appreciated herein that shorter alkylene, alkenylene, and/or alkynylene groups may add less lipophilicity to the compound and accordingly will have different pharmacokinetic behavior. In embodiments of the invention described herein, it is to be understood, in each case, that the recitation of alkylene, alkenylene, and alkynylene refers to alkylene, alkenylene, and alkynylene as defined herein, and optionally lower alkylene, alkenylene, and alkynylene. Illustrative alkyl groups are, but not limited to, methylene, ethylene, n-propylene, isopropylene, n-butylene, isobutylene, sec-butylene, pentylene, 1,2-pentylene, 1,3-pentylene, hexylene, heptylene, octylene, and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylic acid and derivatives thereof” includes the group CO₂H and salts thereof, and esters and amides thereof, and CN.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

Illustrative substituents include, but are not limited to, a radical —(CH₂)ₓZ^{X}, where x is an integer from 0-6 and Z^{X} is selected from halogen, hydroxy, alkanoyloxy, including C₁-C₆ alkanoyloxy, optionally substituted aroyloxy, alkyl, including C₁-C₆ alkyl, alkoxy, including C₁-C₆ alkoxy, cycloalkyl, including C₃-C₈ cycloalkyl, cycloalkoxy, including C₃-C₈ cycloalkoxy, alkenyl, including C₂-C₆ alkenyl, alkynyl, including C₂-C₆ alkynyl, haloalkyl, including C₁-C₆ haloalkyl, haloalkoxy, including C₁-C₆ haloalkoxy, halocycloalkyl, including C₃-C₈ halocycloalkyl, halocycloalkoxy, including C₃-C₈ halocycloalkoxy, amino, C₁-C₆ alkylamino, (C₁-C₆ alkyl)(C₁-C₆ alkyl)amino, alkylcarbonylamino, N—(C₁-C₆ alkyl)alkylcarbonylamino, aminoalkyl, C₁-C₆ alkylaminoalkyl, (C₁-C₆ alkyl)(C₁-C₆ alkyl)aminoalkyl, alkylcarbonylaminoalkyl, N—(C₁-C₆ alkyl)alkylcarbonylaminoalkyl, cyano, and nitro; or Z^{X} is selected from —CO₂R⁴ and —CONR⁵R⁶, where R⁴, R⁵, and R⁶ are each independently selected in each occurrence from hydrogen, C₁-C₆ alkyl, aryl-C₁-C₆ alkyl, and heteroaryl-C₁-C₆ alkyl.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

### EXAMPLES

### Compound Examples

The compounds described herein may be prepared using the process and syntheses described herein, as well as using general organic synthetic methods. In particular, methods for preparing the compounds are described in U.S. patent application publication 2005/0002942, the disclosure of which is incorporated herein by reference.

### Example

General formation of folate-peptides. The folate-containing peptidyl fragment Pte-Glu-(AA)ₙ-NH(CHR₂)CO₂H (3) is prepared by a polymer-supported sequential approach using standard methods, such as the Fmoc-strategy on an acid-sensitive Fmoc-AA-Wang resin (1), as shown in the following Scheme:

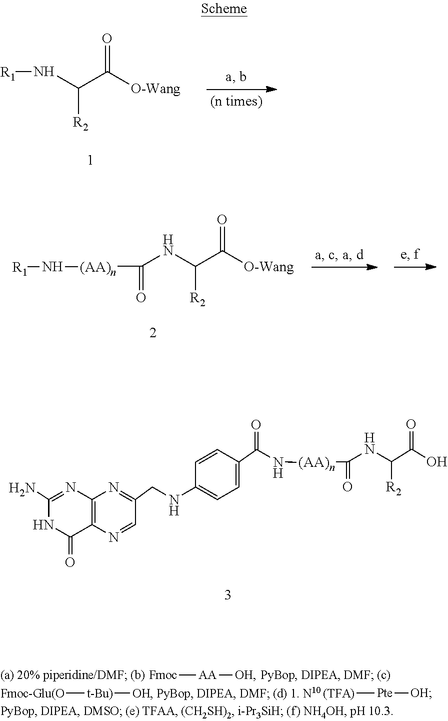

It is to be understood that unnatural amino acids may be included in the foregoing process using the appropriate starting materials.

In this illustrative embodiment of the processes described herein, R₁ is Fmoc, R₂ is the desired appropriately-protected amino acid side chain, and DIPEA is diisopropylethylamine. Standard coupling procedures, such as PyBOP and others described herein or known in the art are used, where the coupling agent is illustratively applied as the activating reagent to ensure efficient coupling. Fmoc protecting groups are removed after each coupling step under standard conditions, such as upon treatment with piperidine, tetrabutylammonium fluoride (TBAF), and the like. Appropriately protected amino acid building blocks, such as Fmoc-Glu-OtBu, Fmoc-D-Glu-OtBu, N¹⁰-TFA-Pte-OH, and the like, are used, as described in the Scheme, and represented in step (b) by Fmoc-AA-OH. Thus, AA refers to any amino acid starting material, that is appropriately protected. It is to be understood that the term amino acid as used herein is intended to refer to any reagent having both an amine and a carboxylic acid functional group separated by one or more carbons, and includes the naturally occurring alpha and beta amino acids, as well as amino acid derivatives and analogs of these amino acids. In particular, amino acids having side chains that are protected, such as protected serine, threonine, cysteine, aspartate, and the like may also be used in the folate-peptide synthesis described herein. Further, gamma, delta, or longer homologous amino acids may also be included as starting materials in the folate-peptide synthesis described herein. Further, amino acid analogs having homologous side chains, or alternate branching structures, such as norleucine, isovaline, β-methyl threonine, β-methyl cysteine, β,β-dimethyl cysteine, and the like, may also be included as starting materials in the folate-peptide synthesis described herein.

The coupling sequence (steps (a) & (b)) involving Fmoc-AA-OH is performed “n” times to prepare solid-support peptide (2), where n is an integer and may equal 0 to about 100. Following the last coupling step, the remaining Fmoc group is removed (step (a)), and the peptide is sequentially coupled to a glutamate derivative (step (c)), deprotected, and coupled to TFA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups that may form part of the appropriately-protected amino acid side chain. The TFA protecting group is removed upon treatment with base (step (f)) to provide the folate-containing peptidyl fragment (3).

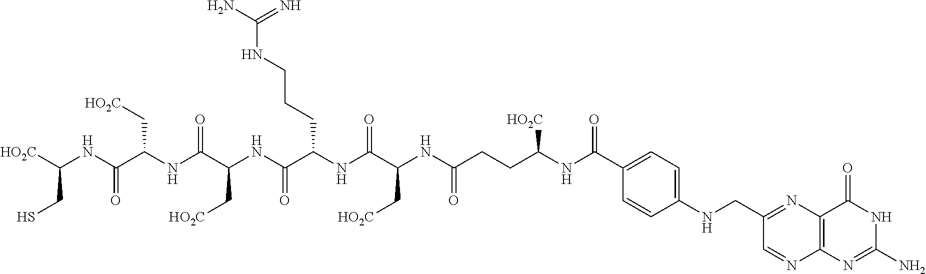 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

### Example

The corresponding compounds containing one or more D-amino acids may also be prepared, such as the following:

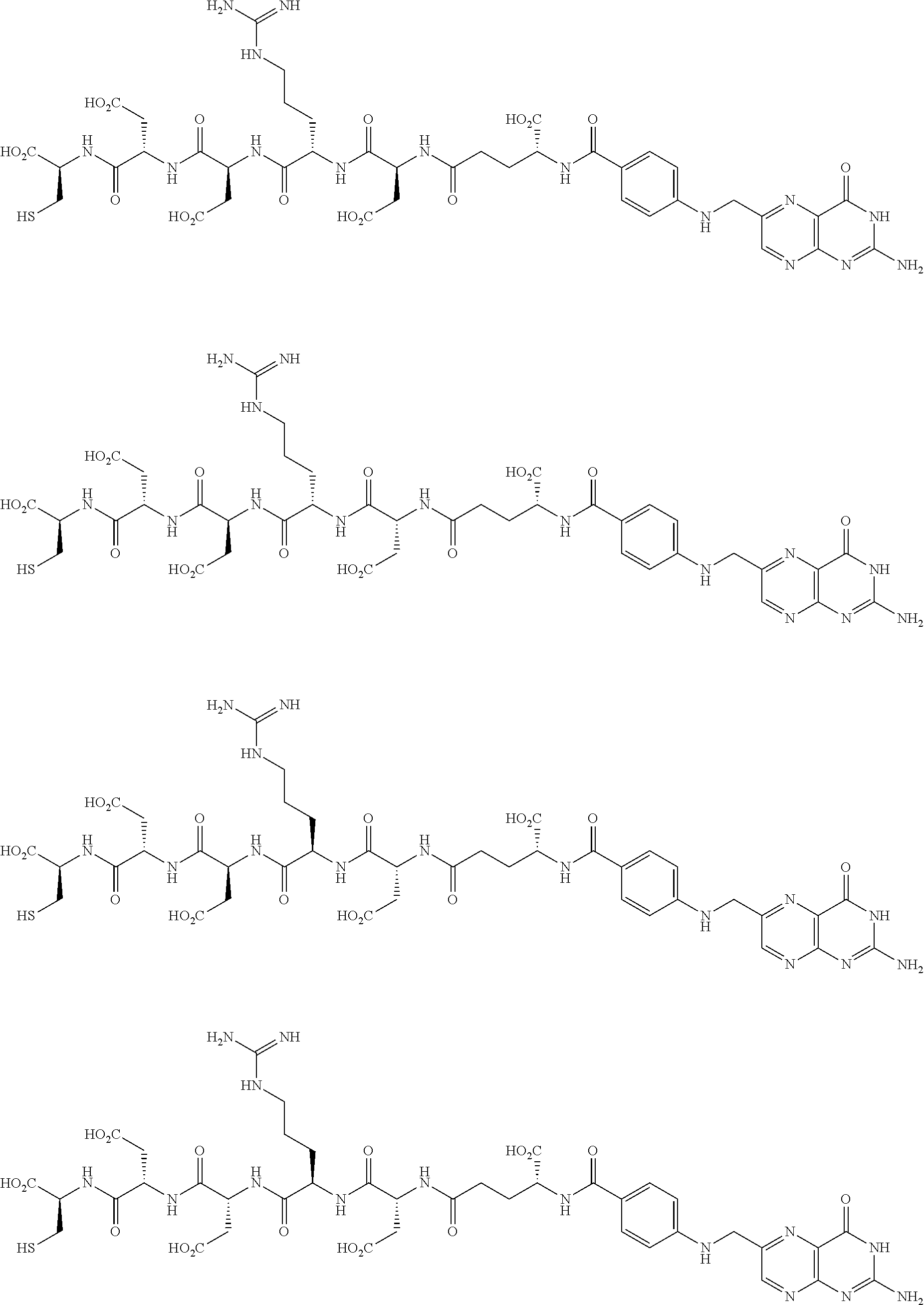

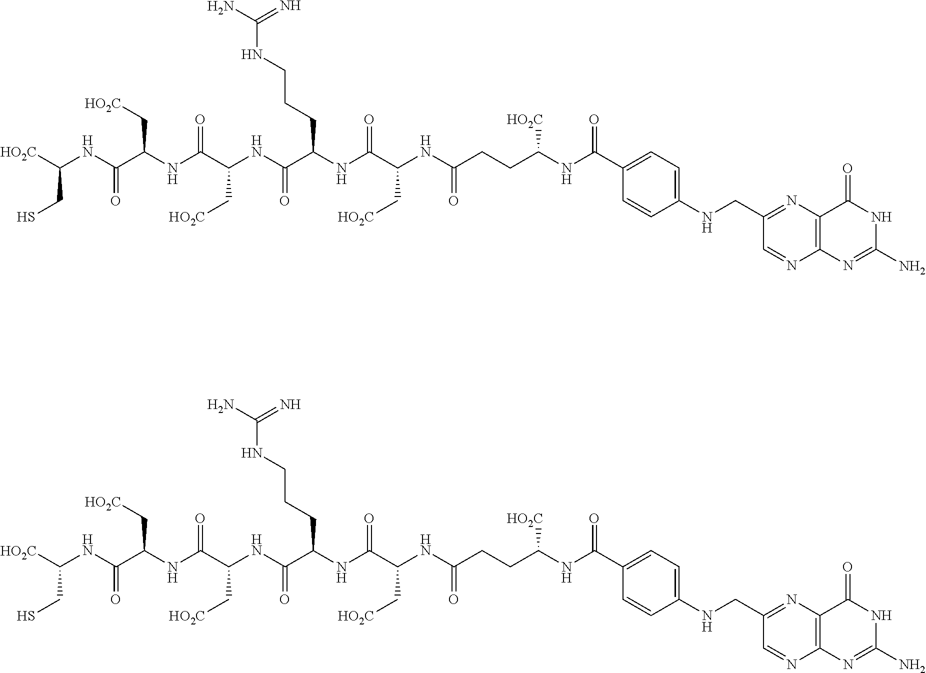 [SMILES: N=C(N)NCCC[C@@H](NC(=O)[C@@H](CC(=O)O)NC(=O)CC[C@@H](NC(=O)c1ccc(NCc2cnc3nc(N)[nH]c(=O)c3n2)cc1)C(=O)O)C(=O)N[C@H](CC(=O)O)C(=O)N[C@H](CC(=O)O)C(=O)N[C@@H](CS)C(=O)O ; N=C(N)NCCC[C@@H](NC(=O)[C@@H](CC(=O)O)NC(=O)CC[C@@H](NC(=O)c1ccc(NCc2cnc3nc(N)[nH]c(=O)c3n2)cc1)C(=O)O)C(=O)N[C@H](CC(=O)O)C(=O)N[C@H](CC(=O)O)C(=O)N[C@H](CS)C(=O)O]

and the like.

### Example

Preparation of tubulysin hydrazides. Illustrated by preparing EC0347. N,N-Diisopropylethylamine (DIPEA, 6.1 μL) and isobutyl chloroformate (3.0 μL) were added with via syringe in tandem into a solution of tubulysin B (0.15 mg) in anhydrous EtOAc (2.0 mL) at −15° C. After stirring for 45 minutes at −15° C. under argon, the reaction mixture was cooled down to −20° C. and to which was added anhydrous hydrazine (5.0 μL). The reaction mixture was stirred under argon at −20° C. for 3 hours, quenched with 1.0 mM sodium phosphate buffer (pH 7.0, 1.0 mL), and injected into a preparative HPLC for purification. Column: Waters XTerra Prep MS C₁₈ 10 μm, 19×250 mm; Mobile phase A: 1.0 mM sodium phosphate buffer, pH 7.0; Mobile phase B: acetonitrile; Method: 10% B to 80% B over 20 minutes, flow rate=25 mL/min. Fractions from 15.14-15.54 minutes were collected and lyophilized to produce EC0347 as a white solid (2.7 mg). The foregoing method is equally applicable for preparing other tubulysin hydrazides by the appropriate selection of the tubulysin starting compound.

### Example

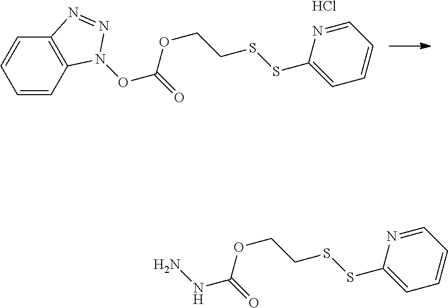 [SMILES: Cl ; O=C(OCCSSc1ccccn1)On1nnc2ccccc21 ; NNC(=O)OCCSSc1ccccn1]

Synthesis of coupling reagent EC0311. DIPEA (0.60 mL) was added to a suspension of HOBt-OCO₂—(CH₂)₂—SS-2-pyridine HCl (685 mg, 91%) in anhydrous DCM (5.0 mL) at 0° C., stirred under argon for 2 minutes, and to which was added anhydrous hydrazine (0.10 mL). The reaction mixture was stirred under argon at 0° C. for 10 minutes and room temperature for an additional 30 minutes, filtered, and the filtrate was purified by flash chromatography (silica gel, 2% MeOH in DCM) to afford EC0311 as a clear thick oil (371 mg), solidified upon standing.

### Example

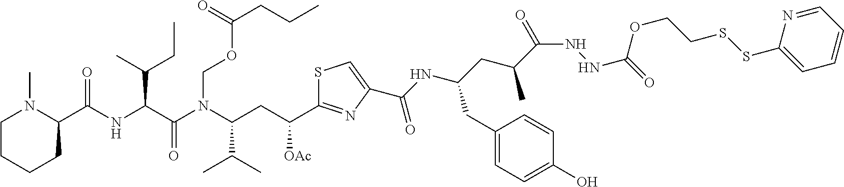 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ccccn2)cs1)C(C)C]

Preparation of tubulysin disulfides (stepwise process). Illustrated for EC0312. DIPEA (36 μL) and isobutyl chloroformate (13 μL) were added with the help of a syringe in tandem into a solution of tubulysin B (82 mg) in anhydrous EtOAc (2.0 mL) at −15° C. After stirring for 45 minutes at −15° C. under argon, to the reaction mixture was added a solution of EC0311 in anhydrous EtOAc (1.0 mL). The resulting solution was stirred under argon at −15° C. for 15 minutes and room temperature for an additional 45 minutes, concentrated, and the residue was purified by flash chromatography (silica gel, 2 to 8% MeOH in DCM) to give EC0312 as a white solid (98 mg). The foregoing method is equally applicable for preparing other tubulysin derivatives by the appropriate selection of the tubulysin starting compound.

### Example

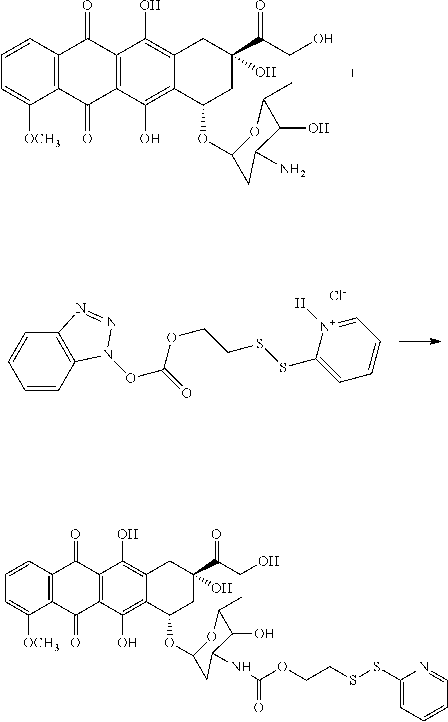 [SMILES: COc1cccc2c1C(=O)c1c(O)c3c(c(O)c1C2=O)C[C@@](O)(C(=O)CO)C[C@@H]3OC1CC(N)C(O)C(C)O1 ; [Cl-] ; O=C(OCCSSc1cccc[nH+]1)On1nnc2ccccc21 ; COc1cccc2c1C(=O)c1c(O)c3c(c(O)c1C2=O)C[C@@](O)(C(=O)CO)C[C@@H]3OC1CC(NC(=O)OCCSSc2ccccn2)C(O)C(C)O1]

Hydroxydaunorubucin pyridyldisulfide. Similarly, this compound was prepared as described herein in 65% yield, and according to the foregoing scheme.

### Example

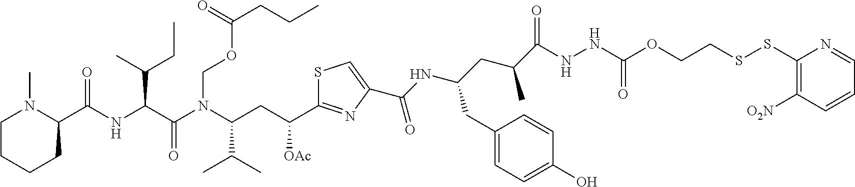 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ncccc2[N+](=O)[O-])cs1)C(C)C]

Tubulysin B pyridyldisulfide. Similarly, this compound is prepared as described herein.

### Example

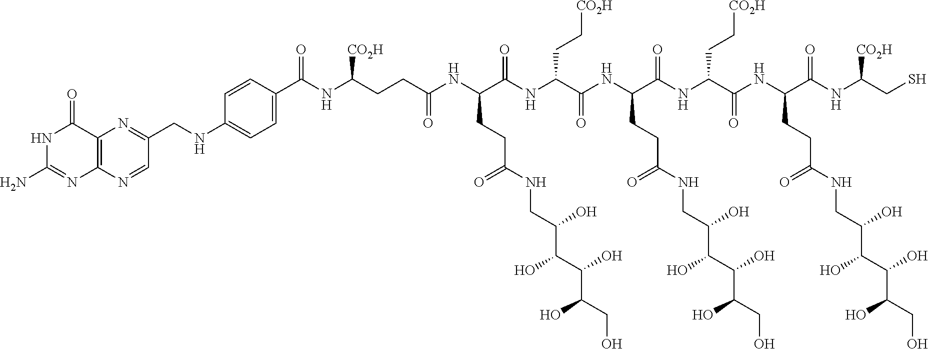

D-EC0488. This compound was prepared by SPPS according to the general peptide synthesis procedure described herein starting from H-Cys(4-methoxytrityl)-2-chlorotrityl-Resin, and the following SPPS reagents:

| Reagents | mmol | equivalent | MW | amount |
|---|---|---|---|---|
| H-D-Cys(4-methoxytrityl)-2- | 0.10 | | | 0.17 g |
| chlorotrityl-Resin | | | | |
| (loading 0.6 mmol/g) | | | | |
| D-EC0475 | 0.13 | 1.3 | 612.67 | 0.082 g |
| Fmoc-D-Glu(OtBu)-OH | 0.19 | 1.9 | 425.47 | 0.080 g |
| D-EC0475 | 0.13 | 1.3 | 612.67 | 0.082 g |
| Fmoc-D-Glu(OtBu)-OH | 0.19 | 1.9 | 425.47 | 0.080 g |
| D-EC0475 | 0.13 | 1.3 | 612.67 | 0.082 g |
| Fmoc-D-Glu-OtBu | 0.19 | 1.9 | 425.47 | 0.080 g |
| N¹⁰TFA-Pteroic Acid | 0.16 | 1.6 | 408.29 | 0.066 g |
| (dissolve in 10 ml DMSO) | | | | |
| DIPEA | | 2.0 eq of AA | | |
| PyBOP | | 1.0 eq of AA | | |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3×(10 min), before each amino acid coupling. Continue to complete all 9 coupling steps. At the end treat the resin with 2% hydrazine in DMF 3×(5 min) to cleave TFA protecting group on Pteroic acid, wash the resin with DMF (3×), IPA (3×), MeOH (3×), and bubble the resin with argon for 30 min.

Cleavage step. Reagent: 92.5% TFA, 2.5% H₂O, 2.5% triisopropylsilane, 2.5% ethanedithiol. Treat the resin with cleavage reagent 3×(10 min, 5 min, 5 min) with argon bubbling, drain, wash the resin once with cleavage reagent, and combine the solution. Rotavap until 5 ml remains and precipitate in diethyl ether (35 mL). Centrifuge, wash with diethyl ether, and dry. About half of the crude solid (−100 mg) was purified by HPLC.

HPLC Purification step. Column: Waters Xterra Prep MS C18 10 μm 19×250 mm; Solvent A: 10 mM ammonium acetate, pH 5; Solvent B: ACN; Method: 5 min 0% B to 25 min 20% B 26 mL/min. Fractions containing the product was collected and freeze-dried to give 43 mg EC0488 (51% yield). ¹H NMR and LC/MS (exact mass 1678.62) were consistent with the product.

### Example

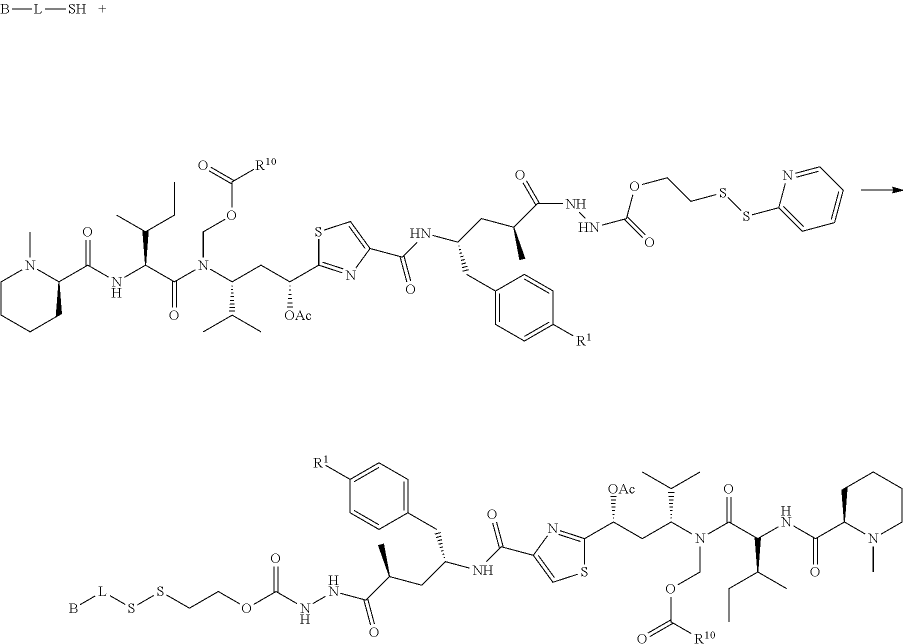

General Synthesis of Disulfide Containing Tubulysin Conjugates. Illustrated with pyridinyl disulfide derivatives of certain naturally occurring tubulysins, where R¹ is H or OH, and R¹⁰, is alkyl or alkenyl. A binding ligand-linker intermediate containing a thiol group is taken in deionized water (ca. 20 mg/mL, bubbled with argon for 10 minutes prior to use) and the pH of the suspension was adjusted by saturated NaHCO₃ (bubbled with argon for 10 minutes prior to use) to about 6.9 (the suspension may become a solution when the pH increased). Additional deionized water is added (ca. 20-25%) to the solution as needed, and to the aqueous solution is added immediately a solution of EC0312 in THF (ca. 20 mg/mL). The reaction mixture becomes homogenous quickly. After stirring under argon, e.g. for 45 minutes, the reaction mixture is diluted with 2.0 mM sodium phosphate buffer (pH 7.0, ca 150 volume percent) and the THF is removed by evacuation. The resulting suspension is filtered and the filtrate may be purified by preparative HPLC (as described herein). Fraction are lyophilized to isolate the conjugates. The foregoing method is equally applicable for preparing other tubulysin conjugates by the appropriate selection of the tubulysin starting compound.

### Example

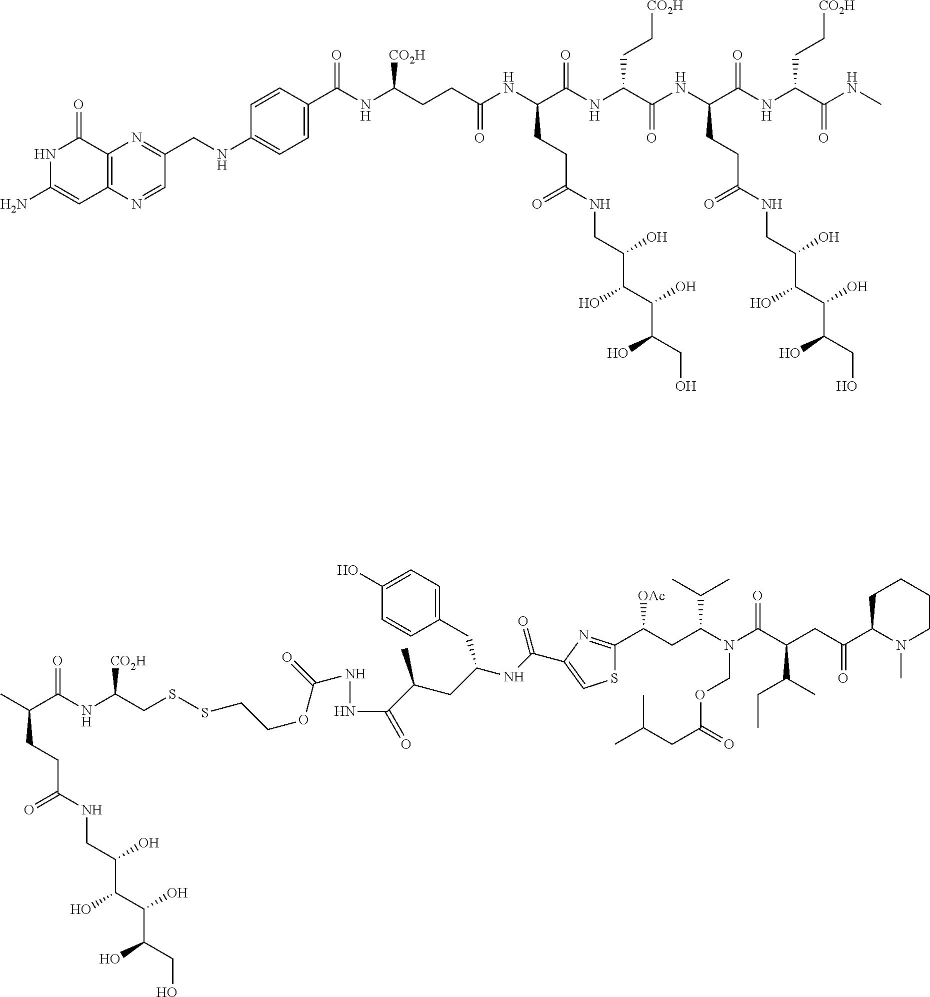

General Method 2 for Preparing Conjugates (one-pot). Illustrated with preparation of EC1456. DIPEA (7.8 μL) and isobutyl chloroformate (3.1 μL) were added with the help of a syringe in tandem into a solution of tubulysin A (18 mg) in anhydrous EtOAc (0.50 mL) at −15° C. After stirring for 35 minutes at −15° C. under argon, to the reaction mixture was added a solution of EC0311 (5.8 mg) in anhydrous EtOAc (0.50 mL). The cooling was removed and the reaction mixture was stirred under argon for an additional 45 minutes, concentrated, vacuumed, and the residue was dissolved in THF (2.0 mL). Meanwhile, D-EC0488 (40 mg) was dissolved in deionized water (bubbled with argon for 10 minutes prior to use) and the pH of the aqueous solution was adjusted to 6.9 by saturated NaHCO₃. Additional deionized water was added to the D-EC0488 solution to make a total volume of 2.0 mL and to which was added immediately the THF solution containing the activated tubulysin. The reaction mixture, which became homogeneous quickly, was stirred under argon for 50 minutes and quenched with 2.0 mM sodium phosphate buffer (pH 7.0, 15 mL). The resulting cloudy solution was filtered and the filtrate was injected into a preparative HPLC for purification. Column: Waters XTerra Prep MS C₁₈ 10 μm, 19×250 mm; Mobile phase A: 2.0 mM sodium phosphate buffer, pH 7.0; Mobile phase B: acetonitrile; Method: 1% B for 5 minutes, then 1% B to 60% B over the next 30 minutes, flow rate=26 mL/min. Fractions from 20.75-24.50 minutes were collected and lyophilized to afford EC1456 as a pale yellow fluffy solid (26 mg). The foregoing method is equally applicable for preparing other tubulysin and other conjugates by the appropriate selection of the tubulysin or other drug starting compound.

### Example

EC1426 is prepared according to the following process.

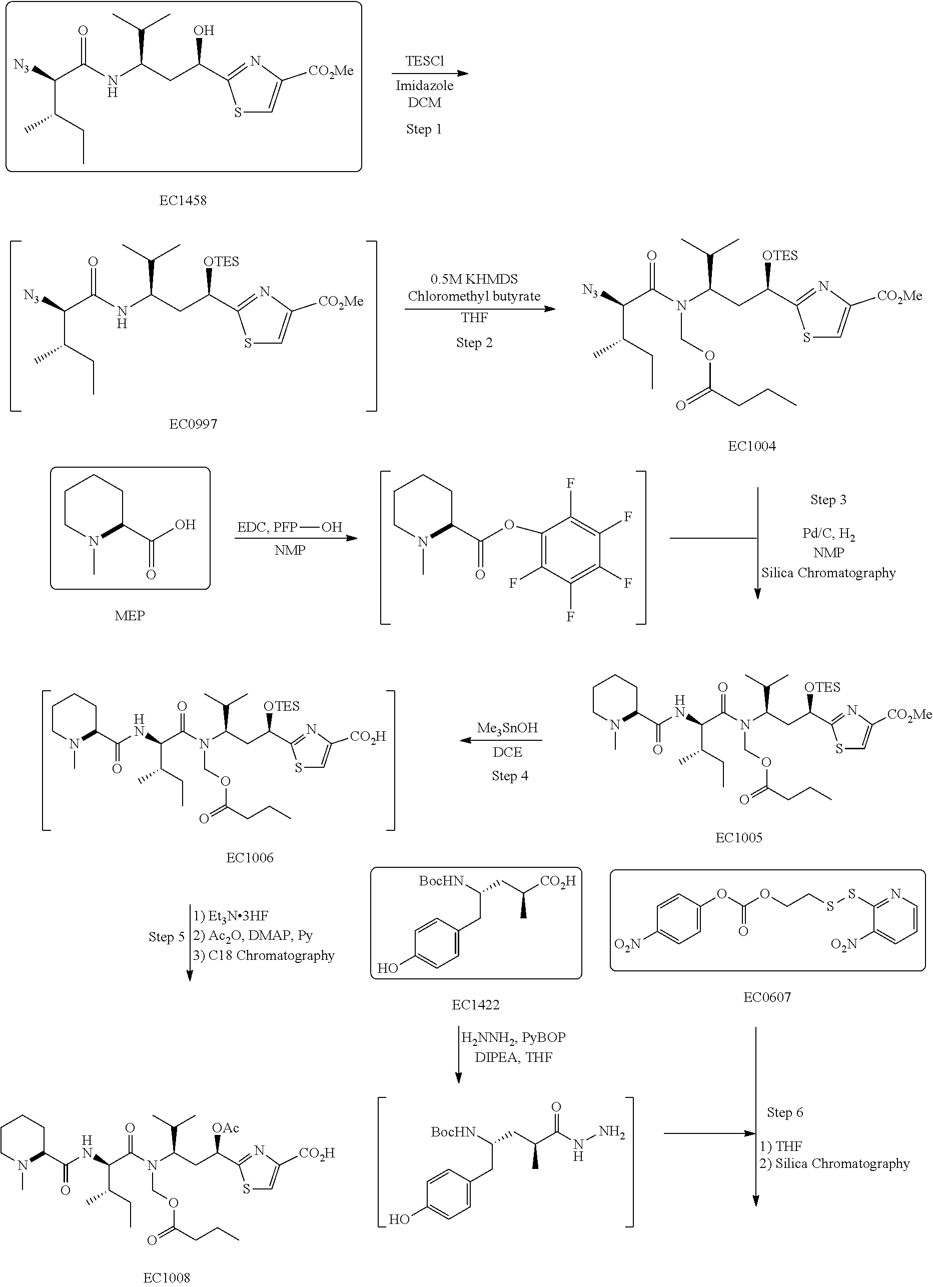

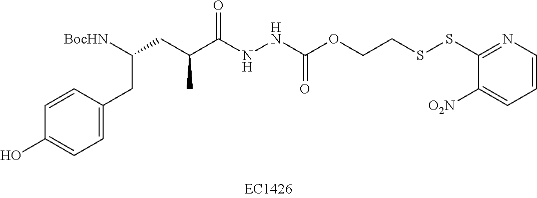 [SMILES: C[C@@H](C[C@H](Cc1ccc(O)cc1)NC(=O)OC(C)(C)C)C(=O)NNC(=O)OCCSSc1ncccc1[N+](=O)[O-]]

### Example

EC1456 is prepared according to the following process.

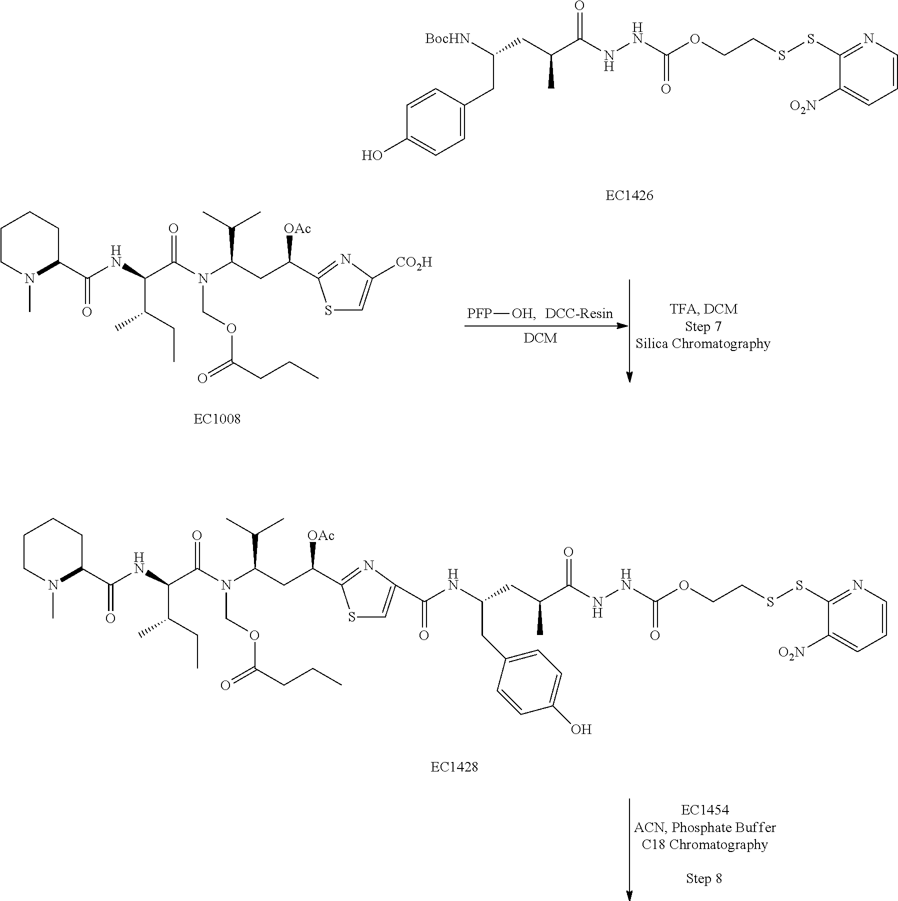

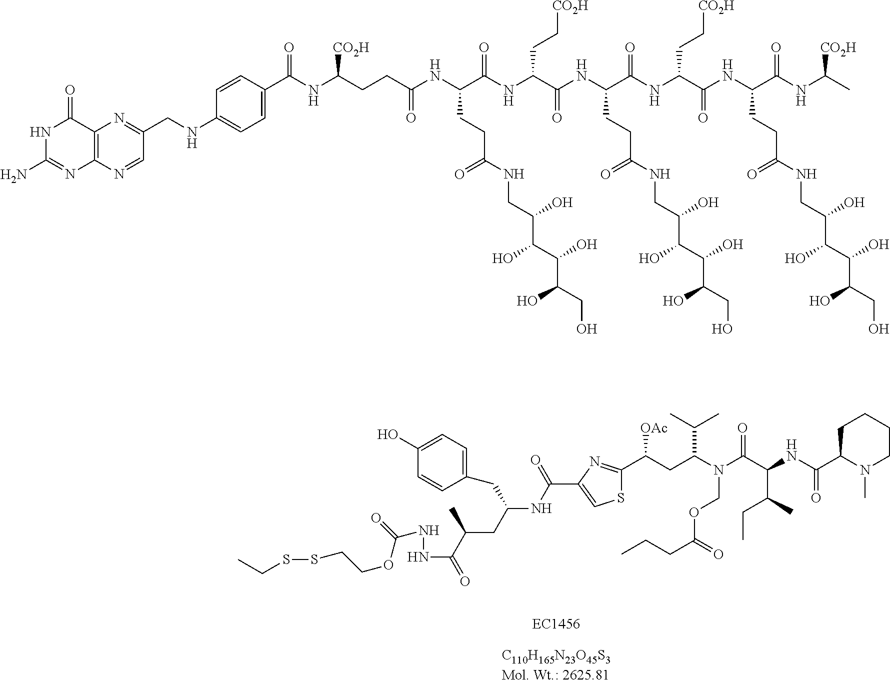 [SMILES: C[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@@H](NC(=O)c1ccc(NCc2cnc3nc(N)[nH]c(=O)c3n2)cc1)C(=O)O)C(=O)O ; CCCC(=O)OCN(C(=O)[C@@H](NC(=O)[C@H]1CCCCN1C)[C@@H](C)CC)[C@H](C[C@@H](OC(C)=O)c1nc(C(=O)N[C@@H](Cc2ccc(O)cc2)C[C@H](C)C(=O)NNC(=O)OCCSSCC)cs1)C(C)C ; "C110H165N23O45S3Mol. Wt.: 2625.81"]

### Example

N¹⁰-TFA Protected EC1454 is prepared according to the following process.

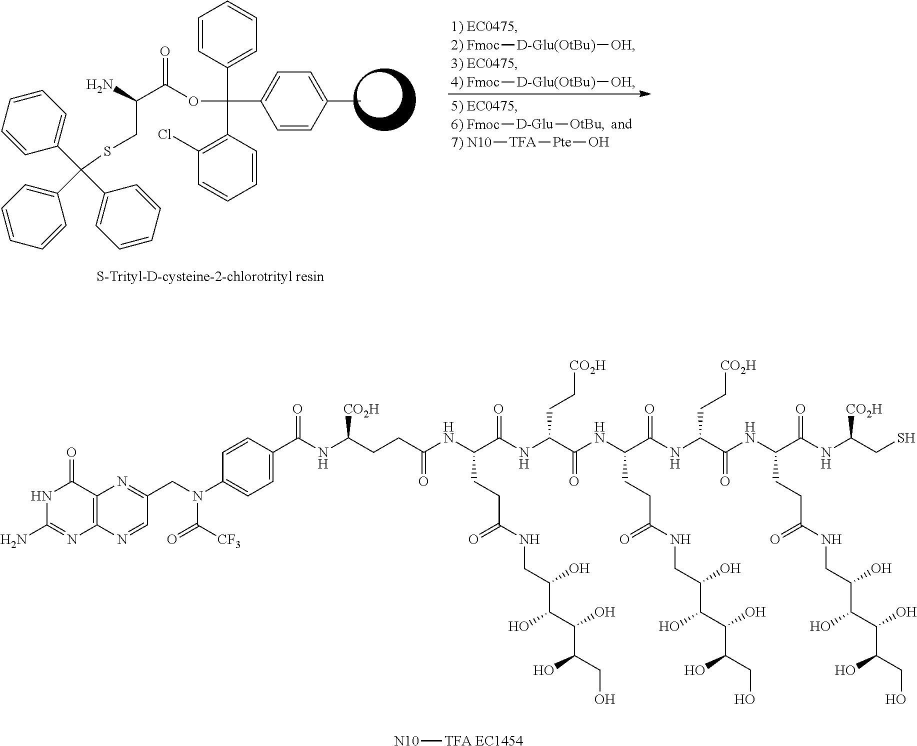

### Example

EC1454 is prepared according to the following process.

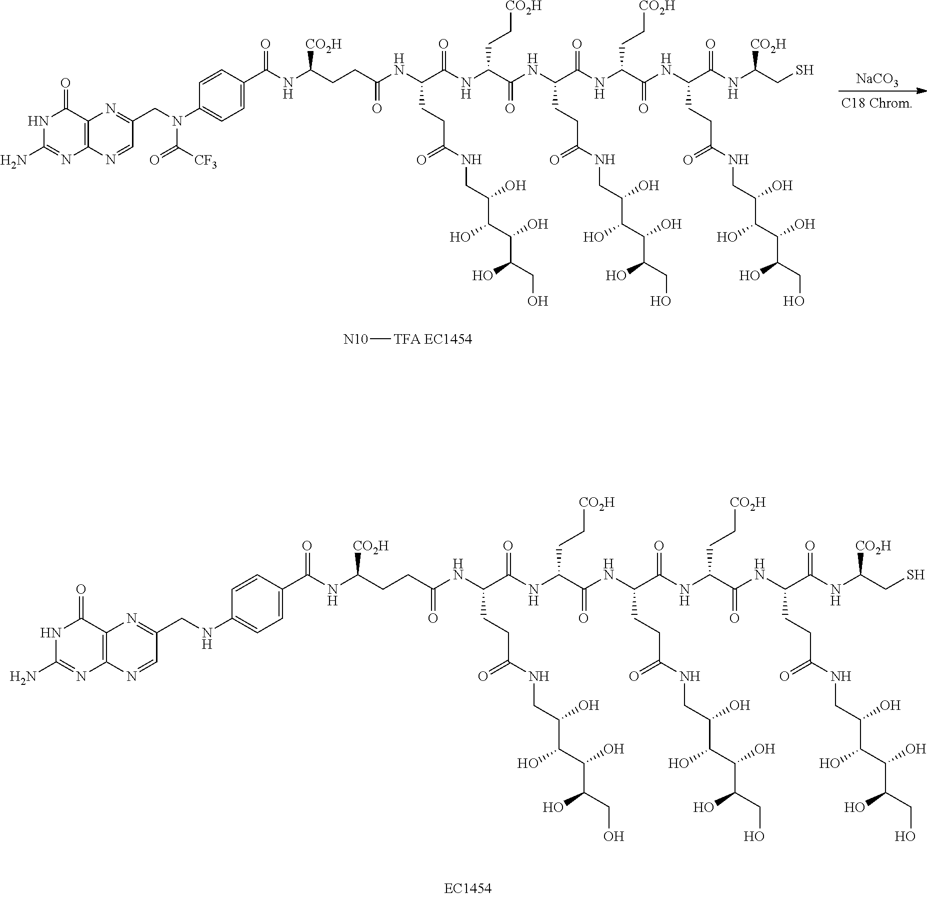

### Example

EC1004 is prepared according to the following process.

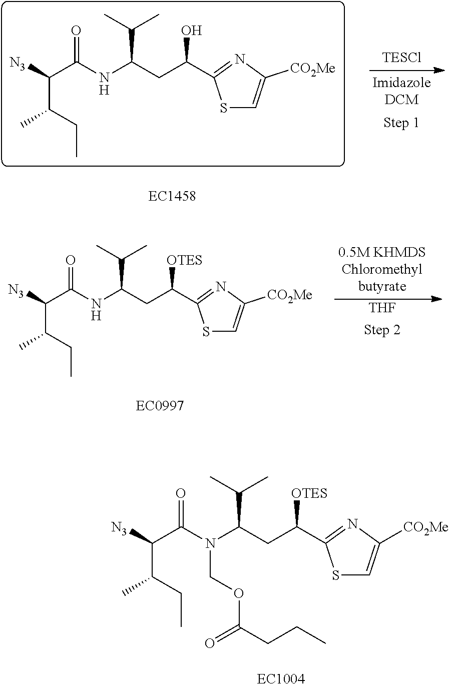

Into a round bottomed flask equipped with magnetic stir bar and temperature probe dipeptide EC1458, imidazole, and methylene chloride is added. Once all the solids have dissolved, the solution is cooled using an ice bath. Chlorotriethylsilane (TESC1) is added drop wise and the ice bath is removed. The reaction is monitored for completion. A second portion of chlorotriethylsilane and/or imidazole is added if necessary. The imidazole HCl salt is removed by filtration and methylene chloride is added. The organics are washed with a saturated solution of sodium chloride (brine), the aqueous layer is back extracted once with methylene chloride, and the combined organic layers are washed with brine. The organic layer is dried over sodium sulfate and concentrated on a rotary evaporator. The residue is dissolved in tetrahydrofuran (THF) and cooled to approximately −45° C. A solution of potassium bis(trimethylsilyl)amide (KHMDS) in toluene is added drop wise. With stirring, chloromethyl butyrate is added and the reaction is monitored. The reaction is quenched with methanol and then ethyl acetate and brine are added. The aqueous layer is discarded and the organics are washed once with brine. The organic layer is concentrated on a rotary evaporator and the oily residue is passed through a short plug of silica gel. The plug is washed with a 20% solution of ethyl acetate in petroleum ether. The combined organics are concentrated on a rotary evaporator until distillation ceases. The crude EC1004 oil is analyzed by LC and NMR and stored in a freezer until use.

### Example

EC1005 is prepared according to the following process.

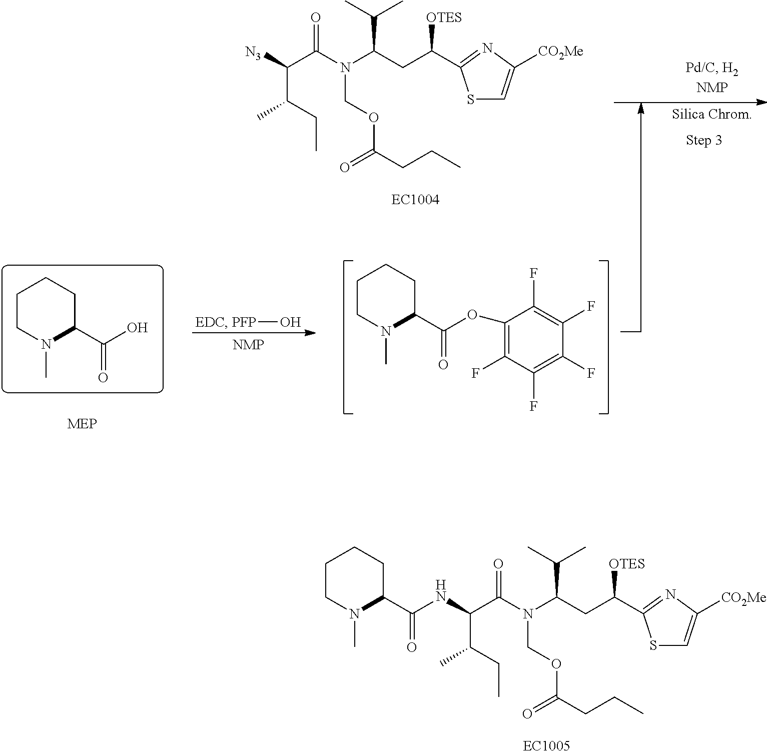

Into an appropriately sized hydrogenation flask place R—N-methyl pipecolinate (MEP), pentafluorophenol, N-methylpyrrolidinone (NMP), and ethyl dimethylaminopropyl carbodiimide (EDC). The mixture is stirred for at least 16 h. EC1004 dissolved in N-methyl pyrrolidinone (NMP) and 10 wt % Pd/C are added. The reaction mixture is stirred/shaken under hydrogen pressure until the reaction is complete by LC analysis. The Pd/C is removed by filtration through celite. The celite is washed with ethyl acetate and the combined organics are washed three times with a 1% sodium bicarbonate/10% sodium chloride solution. The organic layer is dried over sodium sulfate and concentrated on a rotary evaporator. The residue is dissolved in DCM and purified by silica gel chromatography using ethyl acetate and petroleum ether as eluents. Fractions are collected, checked for purity, combined and dried on a rotary evaporator. The EC1005 oil is assayed by LC and stored in a freezer until use.

### Example

EC1008 is prepared according to the following process.

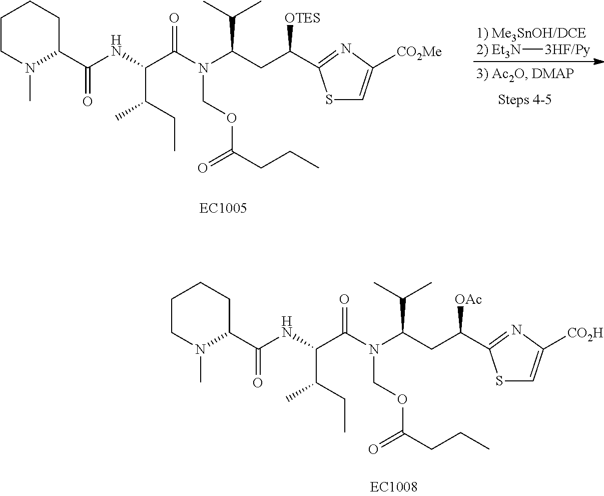

EC1005 is dissolved in 1,2-dichloroethane (DCE) and trimethyltin hydroxide is added. The reaction mixture is heated and reaction is monitored by LC. On completion, the mixture is cooled with an ice bath and filtered. The solids are then washed with DCE. The organic layer is washed once with water and dried over sodium sulfate. The solution is concentrated on a rotary evaporator and the residue dissolved in tetrahydrofuran (THF). Triethylamine trihydrofluoride is added and the mixture stirred while monitoring with LC. Pyridine, dimethylaminopyridine (DMAP), and acetic anhydride are added. The reaction is stirred and monitored by LC. The reaction mixture is concentrated to a residue and the product is purified by C18 column chromatography with acetonitrile and water as eluents. Product fractions are collected, concentrated, and lyophilized to yield a white to off-white powder.

### Example

EC1426 is prepared according to the following process.

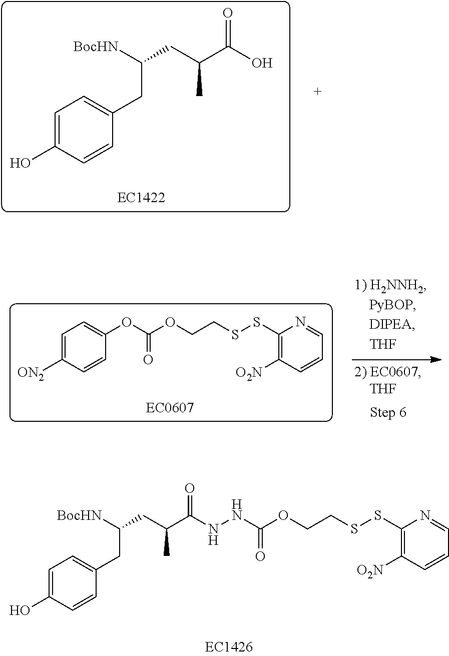

EC1422 is dissolved in tetrahydrofuran (THF) and (Benzotriazol-1-yloxy)tripyrrolidinophosphonium hexafluorophosphate (PyB op) and diisopropylethylamine (DIPEA) are added. Once all the solids have dissolved hydrazine is added and the reaction is stirred and monitored for completion. EC0607 is added and the mixture stirred and monitored for completion by LC. Ethyl acetate is added and the organics are washed once with saturated ammonium chloride, twice with saturated sodium bicarbonate, and once with saturated sodium chloride. The organics are dried over sodium sulfate and concentrated on a rotary evaporator. The crude EC1426 is purified by silica column chromatography with dichloromethane and methanol as eluents. Fractions are collected and the combined product fractions are concentrated on a rotary evaporator to yield a yellow solid.

### Example

EC1428 is prepared according to the following process.

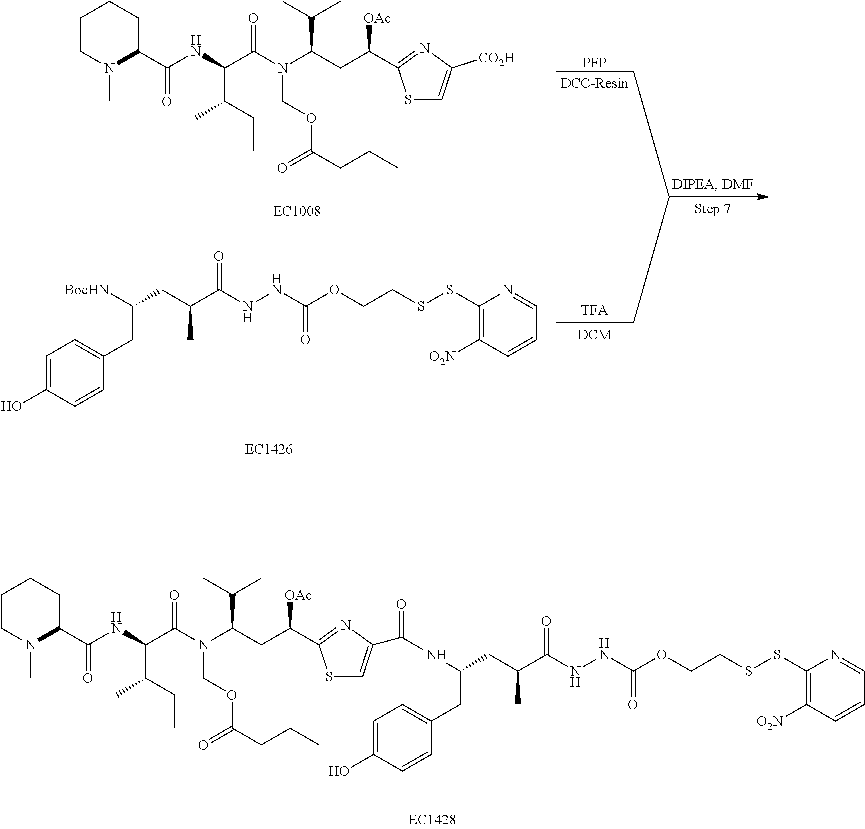

EC1008 is dissolved in dichloromethane and pentafluorophenol dissolved in DCM along with N-cyclohexylcarbodiimide,N′-methyl polystyrene (DCC-resin) are added. The mixture is stirred and reaction completion is monitored by LC. The mixture is filtered to remove the resin and the organic layer is concentrated on a rotary evaporator to yield activated EC1008. In a separate flask, EC1426 is dissolved in dichloromethane and trifluoroacetic acid is added. The reaction mixture is stirred and monitored for completion by LC. The reaction mixture is concentrated on a rotary evaporator to yield deprotected EC1426. The activated EC1008 is dissolved in DMF and diisopropylethylamine (DIPEA) is added. The deprotected EC1426 is dissolved in DMF and added to the reaction mixture. The reaction is stirred and monitored for completion by LC. Ethyl acetate is added and the organics are washed three times with saturated aqueous sodium chloride. The organic layer is dried over sodium sulfate and the volatiles removed by rotary evaporation. The crude EC1428 is purified by silica column chromatography using dichloromethane and methanol as eluents. Fractions are collected, checked for purity, and the combined product fractions are concentrated by rotary evaporation to yield a yellow solid. The EC1428 is stored in a freezer.

### Example

EC1454 is prepared according to the following process.

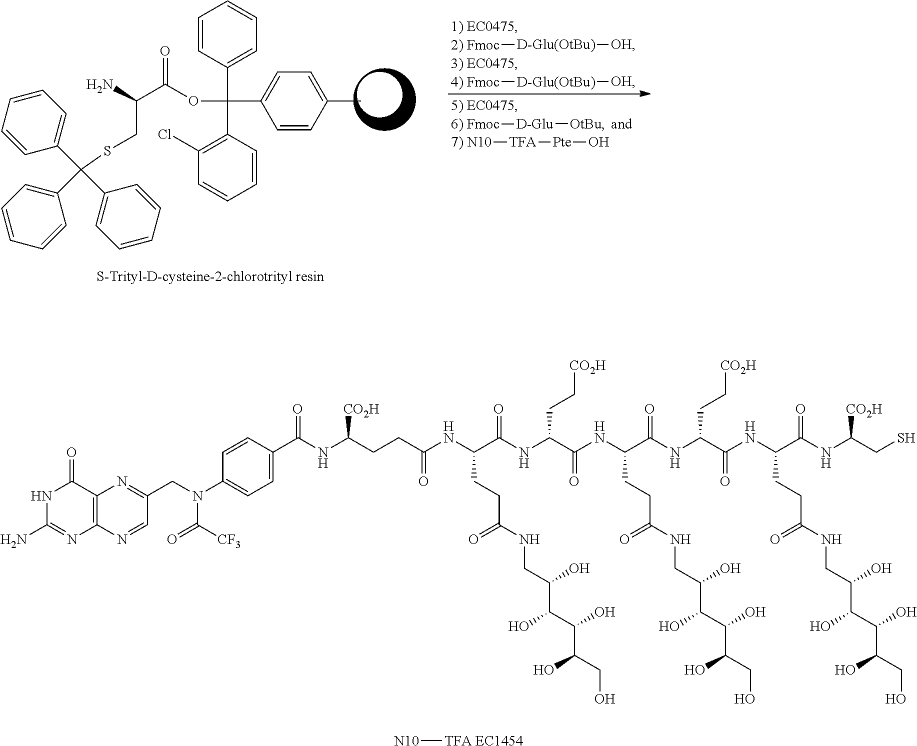

The solid phase synthesis of N¹⁰-TFA protected EC1454 starts with resin bound trityl protected D-cysteine. The resin is suspended in dimethylformamide (DMF) and washed twice with DMF. EC0475 (glucamine modified L-glutamic acid), (Benzotriazol-1-yloxy)tripyrrolidinophosphonium hexafluorophosphate (PyBOP), and diisopropylethylamine (DIPEA) are added to reaction mixture. After at least 1 hour, a Kaiser test is performed to ensure the coupling is complete. The resin is washed three times with DMF, three times with IPA, and three times with DMF. The resin is slowly washed three times with piperidine in DMF, three times with DMF, and three times with IPA. A Kaiser test is performed to confirm deprotection. The resin is washed three times with DMF and the next amino acid in the sequence is coupled following the same process. Monomers are coupled in the following order: 1) EC0475, 2) Fmoc-D-Glu(OtBu)-OH, 3) EC0475, 4) Fmoc-D-Glu(OtBu)-OH, 5) EC0475, 6) Fmoc-D-Glu-OtBu, and 7) N¹⁰-TFA-Pte-OH.

Once the final coupling is complete, the resin is washed three times with methanol and dried by passing argon through the resin at room temperature. The dried resin is suspended in a mixture of TFA, water, ethanedithiol, and triisopropylsilane. After 1 hour the resin is removed by filtration and washed with TFA. The product is precipitated by addition to cold ethyl ether, filtered, and washed with ether. The solids are dried under vacuum at room temperature and stored in a freezer.

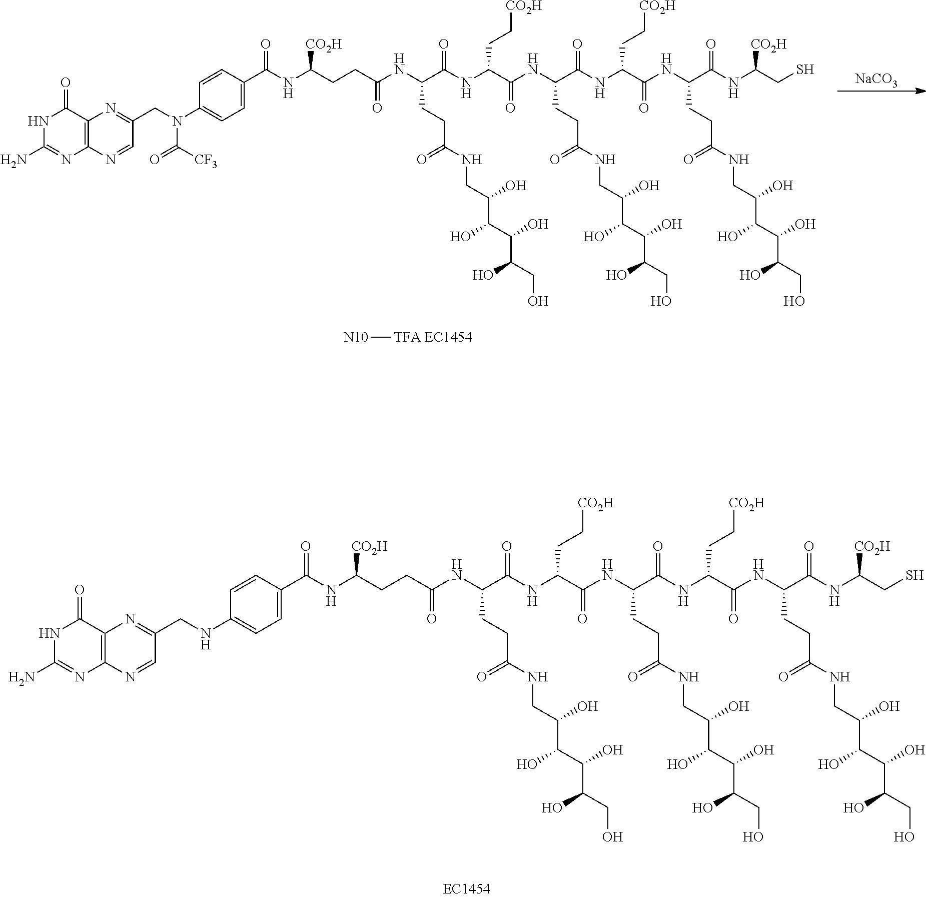

N¹⁰-TFA EC1454 is dissolved in argon sparged water. Sodium carbonate (1M in water, argon sparged) is added to achieve a pH of 9.4-10.1. The reaction mixture is stirred for at least 20 minutes. Once the reaction is complete as determined by LC, it is quenched by adjusting the pH to 1.9-2.3 with 2M HCl. The product is purified by C18 column chromatography using acetonitrile and pH 5 ammonium acetate buffer as eluents. Fractions are collected and checked for purity by HPLC. The combined product fractions are concentrated on a rotary evaporator and then lyophilized to yield EC1454 as a yellow solid. The product is stored at −20° C.

### Example

EC1456 is prepared according to the following process.

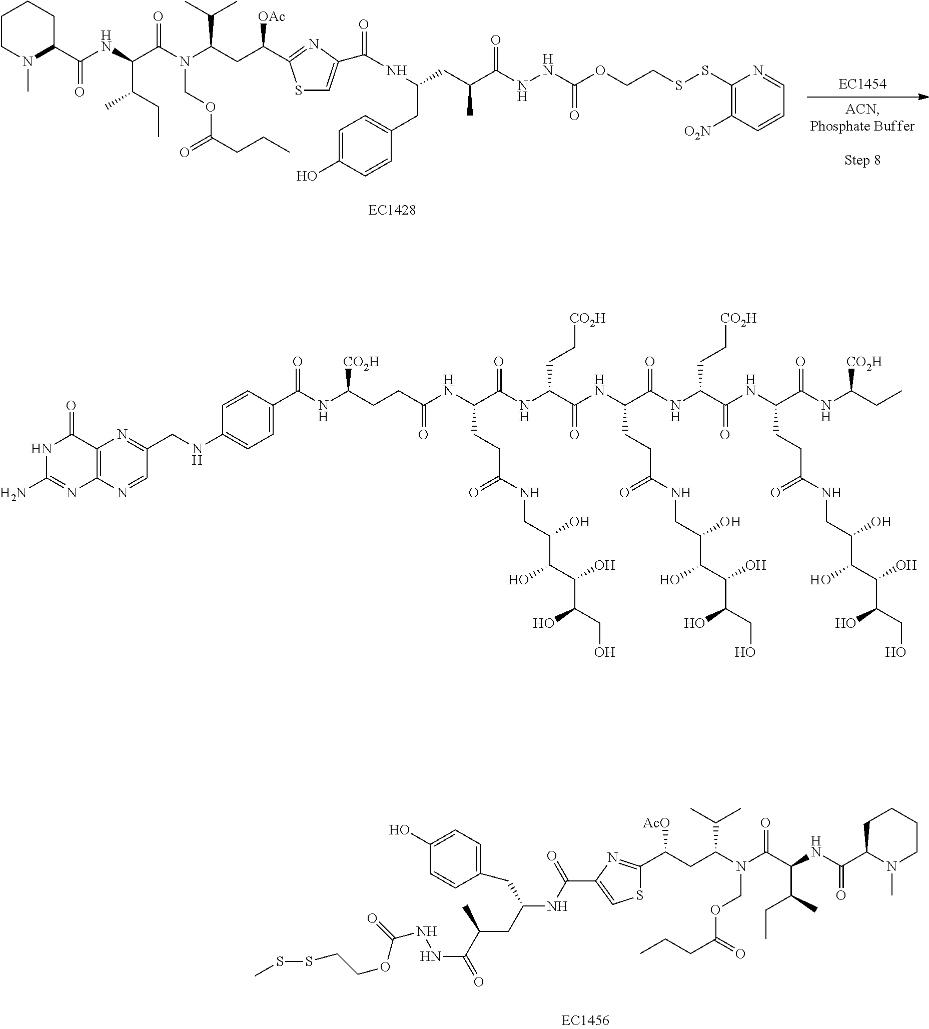

EC1428 is dissolved in acetonitrile and a solution of EC1454 in pH 7.4 Sodium phosphate buffer is added. The solutions are sparged with argon before and after addition. The reaction mixture is stirred for at least 15 minutes and then checked for completion. The desired product is purified by C18 column chromatography using acetonitrile and pH 7.4 phosphate buffer as eluents. The product fractions are collected, checked for purity, combined and concentrated by ultra-filtration to yield an aqueous solution that is 10-20 mg/mL EC1456. The final product solution is sampled for assay and then stored in a freezer.

The positive electrospray mass spectrum of EC1456 was obtained on a high resolution Waters Acquity UPLC Xevo Gs-S QTOF mass spectrometer. The spectrum was obtained following separation of the major component on a UPLC inlet system, the resolving power was approximately 35,000. The accurate mass measurement of the M+H monoisotopic peak was 2625.0598, which is 1.1 ppm error difference from the theoretical value of 2625.0570 for an ion of formula C₁₁₀H₁₆₆N₂₃O₄₅S₃. The isotopic distribution is also consistent with that formula.

Mass spectral features of the ES+ spectrum for EC1456

| Observed Ion | Interpretation |
|---|---|
| 2626.06 | ¹³C isotope of the (M + H)⁺ ion for the MW 2624 |
| | drug substance |
| 1313.54 | ¹³C isotope of the (M + 2H)⁺⁺ ion for the MW 2624 |
| | drug substance |
| 1150.43 | ¹³C isotope of the (M + 2H − 326)⁺⁺ fragment, |
| | corresponding to the cleavage of the peptide bond |
| | at the tertiary nitrogen and the loss of the |
| | butyric acid moiety. |
| 876.03 | ¹³C isotope of the (M + 3H)⁺⁺⁺ ion for the MW 2624 |
| | drug substance |
| 657.27 | ¹³C isotope of the (M + 4H)⁺⁺⁺⁺ ion for the MW 2624 |
| | drug substance |

A sample of ˜30 mg EC1456 was dissolved in 665 μL of a 9:1 mixture of deuterated dimethylsulfoxide and deuterated water. The ¹H NMR spectrum was obtained at 500 MHz at 26 deg. C. on an Agilent model DD2 spectrometer fitted with a 2 channel probe containing both broadband and proton observe coils. The ¹³C NMR spectrum was obtained at 125 MHz on the same instrument under identical conditions. All spectra were referenced to the DMSO solvent residual signals at 2.5 ppm (¹H) and 39.50 ppm (¹³C).

All spectral features are assigned for both NMR spectra in the tables below (¹H and ¹³C) using the atom numbering in the following figure (where the * symbols indicates the connection for the disulfide bond).

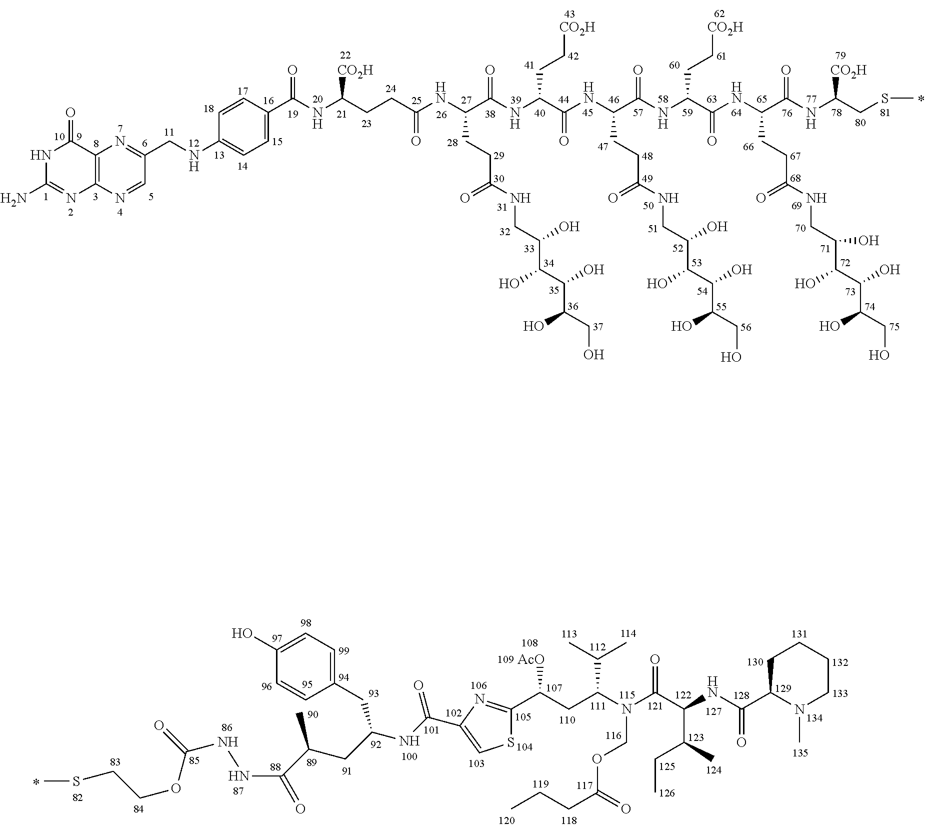

Assignments were made on the basis of both 1D and 2D NMR experiments, including through bond H—H connectivity using the COSY and TCSY 2D experiments, through space H—H proximity using 2D NOESY, carbon multiplicity measurement using the 1D DEPT experiment and through bond C—H connectivity using the proton detected 2D experiments HSQC and HMBC. In most cases of overlap in the 1D spectra (different protons or carbons resonating at the same chemical shift) could be resolved in the 2D spectra, in these cases the tables reflect the chemical shifts measured from the 2D spectra but summed integrations for the group of co-resonating species. In some cases of 1D overlap (such as the nearly identical glutamic acid and glucamine subunits) there was also overlap in the 2D correlation spectra which precludes unambiguous assignment of single or multiple resonances between multiple atom numbers, in these cases there are multiple entries for chemical shift and/or atom number assignments in a single table row.

NH and OH protons were exchanged by the D₂O deuterium atoms and are mostly absent from the spectrum, except weak broad peaks in the 5-10 ppm region. The ¹H peaks in the spectrum that are not listed in the table include a broad HOD peak at 3.75 ppm, and a DMSO peak at 2.50 ppm. The HOD peak does not obscure any resonances, but elevates the integrations for nearby resonances at 4.2 and 3.4-3.7 ppm due to the broad baseline rise. The DMSO peak obscures the resonance for H129, which is not integrated for this reason. The ¹³C peaks in spectrum not listed in the table include the very large DMSO solvent at 39.50 ppm. The DMSO peak obscures both the signals from C91 and C93. The C116 peak is not observable in the ¹³C spectrum due to extensive broadening due to conformational changes around the nearby amide group. All three chemical shifts (C91, C93, C116) are visible in and measured in the proton detected 2D correlation spectra.

Proton NMR assignments for EC1456

| Proton | | |
|---|---|---|
| Chemical Shift (ppm) | Assignment | # protons |
| 8.61 | 5 | 1 |
| 8.16 | 103 | 1 |
| 7.58 | 15, 17 | 2 |
| 6.96 | 95, 99 | 2 |
| 6.62 | 14, 18 | 4 |
| 6.59 | 96, 98 | |
| 6.18 | 116 Ha | 1 |
| 5.7 | 107 | 1 |
| 5.24 | 116 Hb | 1 |
| 4.47 | 11 | 2 |
| 4.39 | 111, 122 | 2 |
| 4.21 | 78 | 10 |
| 4.21 | 65 | |
| 4.18 | 84 | |
| 4.15 | 46 | |
| 4.15 | 59 | |
| 4.13 | 21 | |
| 4.13 | 40 | |
| 4.09 | 27 | |
| 4.09 | 92 | |
| 3.61 | 33, 52, 71 | 3 |
| 3.56 | 34, 53, 72 | 6 |
| 3.54 | 37Ha, 56Ha, 75Ha | |
| 3.46 | 36, 55, 74 | 3 |
| 3.4 | 35, 54, 73 | 6 |
| 3.38 | 37Hb, 56Hb, 75Hb | |
| 3.21 | 80Ha, 32Ha, 51 Ha, | 4 |
| | 70 Ha | |
| 3.05 | 32Hb, 51Hb, 70Hb | 3 |
| 2.93 | 80 Hb | 3 |
| 2.91 | 83 | |
| 2.8 | 133Ha | 1 |
| 2.68 | 93 | 2 |
| 2.49 (see text) | 129 | 1 |
| 2.35 | 89 | 2 |
| 2.33 | 110Ha | |
| 2.8 | 133Hb | 37 |
| 2.17 | 118 | |
| 2.14-2.08 | 24, 29, 42, 48, 61, 67 | |
| 2.09 | 110Hb | |
| 2.08 | 109 | |
| 2.02 | 135 | |
| 1.97-1.70 | 28, 41, 47, 60, 66 | |
| 1.92 | 23Ha | |
| 1.88 | 123 | |
| 1.8 | 91Ha | |
| 1.79 | 23Hb | |
| 1.77 | 112 | |
| 1.6 | 131Ha | 9 |
| 1.56 | 130Ha | |
| 1.5 | 132Ha | |
| 1.5 | 91Hb | |
| 1.45 | 125Ha | |
| 1.42 | 119 | |
| 1.4 | 132Hb | |
| 1.33 | 130Hb | |
| 1.14 | 131Hb | 2 |
| 1.07 | 125Hb | |
| 1 | 90 | 3 |
| 0.94 | 114 | 3 |
| 0.79 | 124 | 3 |
| 0.77 | 126 | 3 |
| 0.75 | 120 | 3 |
| 0.64 | 113 | 3 |

Carbon NMR assignments for EC1456

| Carbon | |
|---|---|
| Chemical shift | |
| (ppm) | Assignment |
| 176.77, 176.32 | 43, 62 |
| 175.74 | 88 |
| 175.42 | 22 |
| 174.75 | 121 |
| 173.87, 172.68, | 25, 38, 44, 57, 63 |
| 172.15, 171.94, | |
| 171.84 | |
| 173.43 | 79 |
| 173.3 | 128 |
| 172.79 (2x), 172.72 | 30, 49, 68 |
| 172.46 | 117 |
| 170.87 | 76 |
| 170.39 | 108 |
| 169.3 | 105 |
| 166.09 | 19 |
| 162.4 | 9 |
| 160.7 | 101 |
| 156.4 | 85 |
| 156.09 | 3 |
| 155.71 | 97 |
| 154.59 | 1 |
| 150.84 | 13 |
| 149.63 | 102 |
| 149.11 | 6 |
| 148.99 | 5 |
| 130.44 | 95, 99 |
| 128.99 | 15, 17 |
| 128.89 | 94 |
| 127.99 | 8 |
| 124.97 | 103 |
| 122.24 | 16 |
| 115.25 | 96, 98 |
| 111.86 | 14, 18 |
| 72.17 (3x) | 35, 54, 73 |
| 71.78, 71.74, 71.71 | 33, 52, 71 |
| 71.62, 71.59 (2x) | 36, 55, 74 |
| 69.65, 69.57 (2x) | 34, 53, 72 |
| 69.45 | 107 |
| 69.34 | 116 |
| 68.51 | 129 |
| 63.42 (3x) | 37, 56, 75 |
| 63.03 | 84 |
| 55.08 | 133 |
| 54.05 | 40 |
| 53.88 | 78 |
| 53.46 (2x) | 46, 59 |
| 53.33 | 27 |
| 52.96 (2x) | 122, 111 |
| 52.89 | 21 |
| 52.55 | 65 |
| 49.77 | 92 |
| 46.07 | 11 |
| 44.02 | 135 |
| 42.85 | 80 |
| 42.34 (2x), 42.29 | 32, 51, 70 |
| 39.52 | 93 |
| 38.95 | 91 |
| 37.43 | 83 |
| 35.95 | 118 |
| 35.43 | 123 |
| 35.38 | 89 |
| 34.86 | 110 |
| 32.56, 32.36, | 24, 29, 42, 48, 61, 67 |
| 32.16, 32.09 (2x), | |
| 31.81 | |
| 30.5 | 112 |
| 29.95 | 130 |
| 28.60, 28.04, 27.78 | 28, 41, 47, 60, 66 |
| (2x), 27.66 | |
| 27 | 23 |
| 25.01 | 132 |
| 24.43 | 125 |
| 23.04 | 131 |
| 20.86 | 109 |
| 20.56 | 114 |
| 19.64 | 113 |
| 18.36 | 90 |
| 18.04 | 119 |
| 15.64 | 124 |
| 13.72 | 120 |
| 10.28 | 126 |

The IR spectrum of EC1456 was acquired on a Nexus 6700® Fourier transform infrared (FT-IR) spectrophotometer (Thermo Nicolet) equipped with an Ever-Glo mid/far IR source, an extended range potassium bromide (KBr) beam splitter, and a deuterated triglycine sulfate (DTGS) detector. An attenuated total reflectance (ATR) accessory (Thunderdome™, Thermo Spectra-Tech), with a germanium (Ge) crystal was used for data acquisition. The spectrum represents 256 co-added scans collected at a spectral resolution of 4 cm⁻¹. A background data set was acquired with a clean Ge crystal. A Log 1/R (R=reflectance) spectrum was acquired by taking a ratio of these two data sets against each other. Wavelength calibration was performed using polystyrene.

Infrared band assignments for EC1456 reference substance

| Characteristic Absorption(s) | |
|---|---|
| (cm⁻¹) | Functional Group |
| 1700-1500 (m, m) | Aromatic C═C Bending |
| 2950-2850 (m or s) | Alkyl C—H Stretch |
| ~3030 (v) | Aromatic C—H Stretch |
| 3550-3200 (broad, s) | Alcohol/Phenol O—H Stretch |
| 3700-3500 (m) | Amide C═O Stretch |

The ultraviolet spectrum EC 1456 acquired on a Perkin-Elmer Lambda 25 UV/Vis spectrometer. The spectrum was recorded at 40.7 uM in 0.1M NaOH solvent on a 1 cm path-length cell at 25 deg. C. The local maxima at 366 nm, 288 nm and 243 nm are due primarily to the Pteroic acid, benzamide/phenol and thiazole-amide substructures, respectively, although the molecule contains dozens of chromaphores with overlapping absorption in the UV region.

### Example

The following additional compounds are described and are prepared according to the general processes described herein.

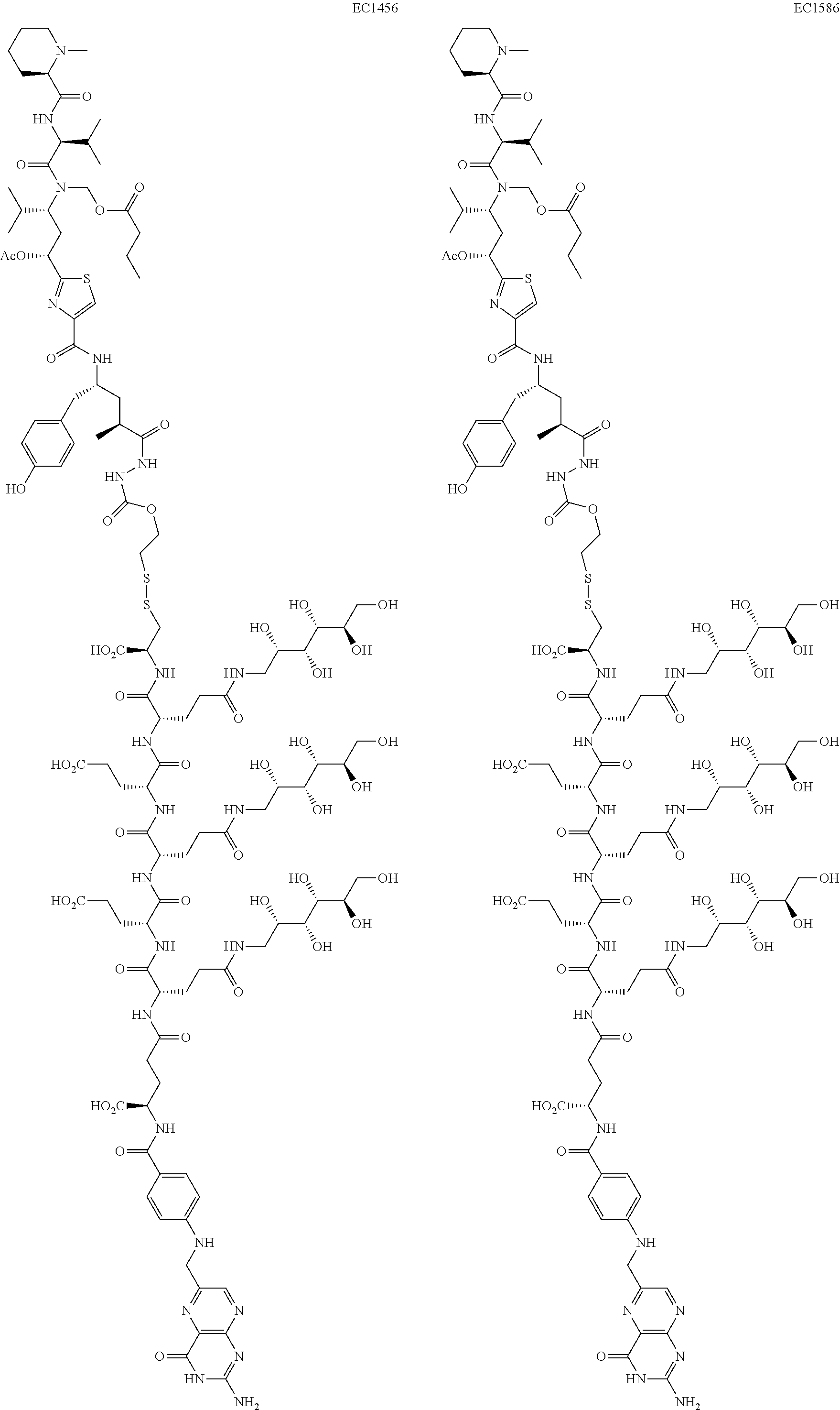

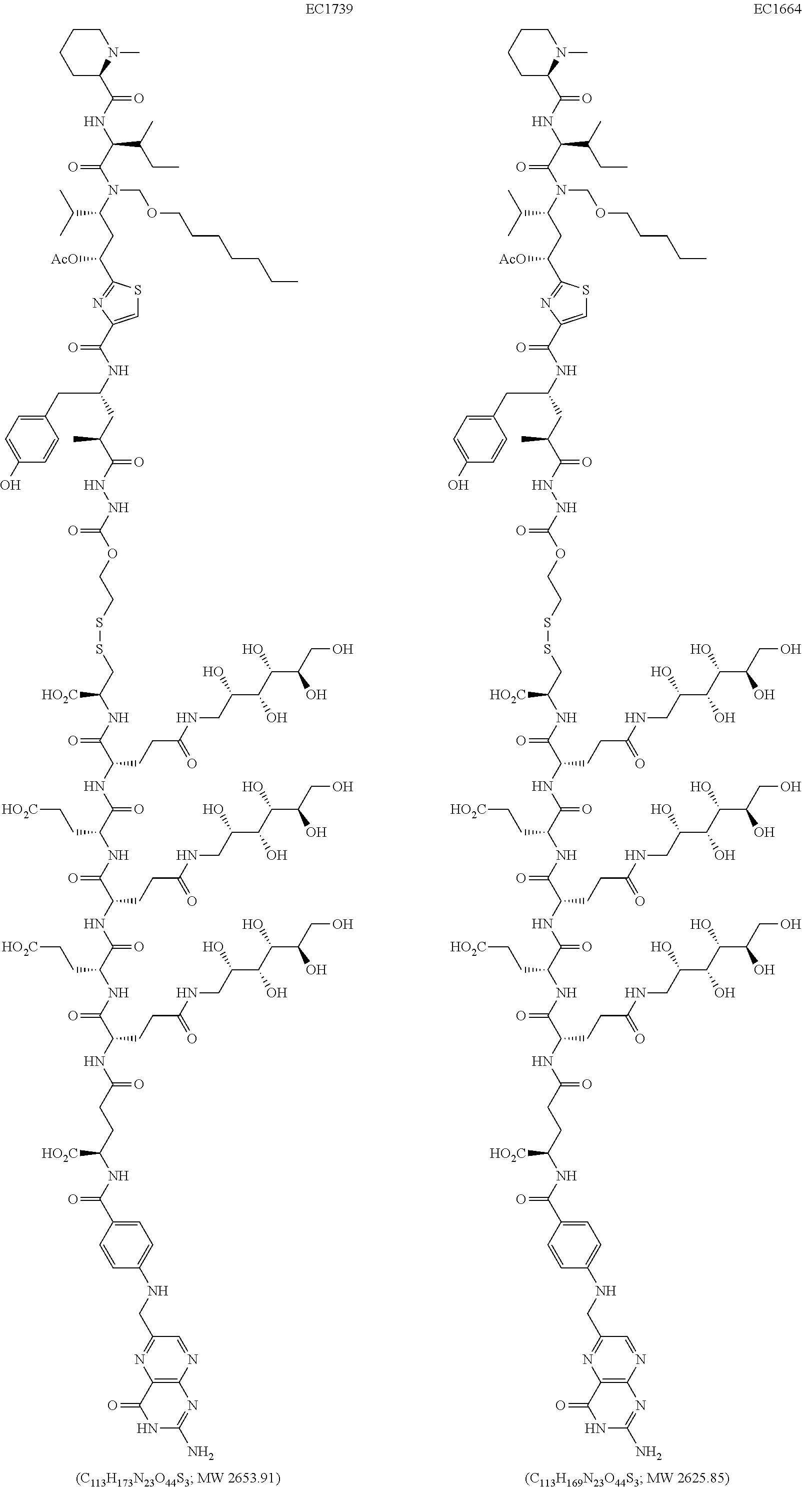

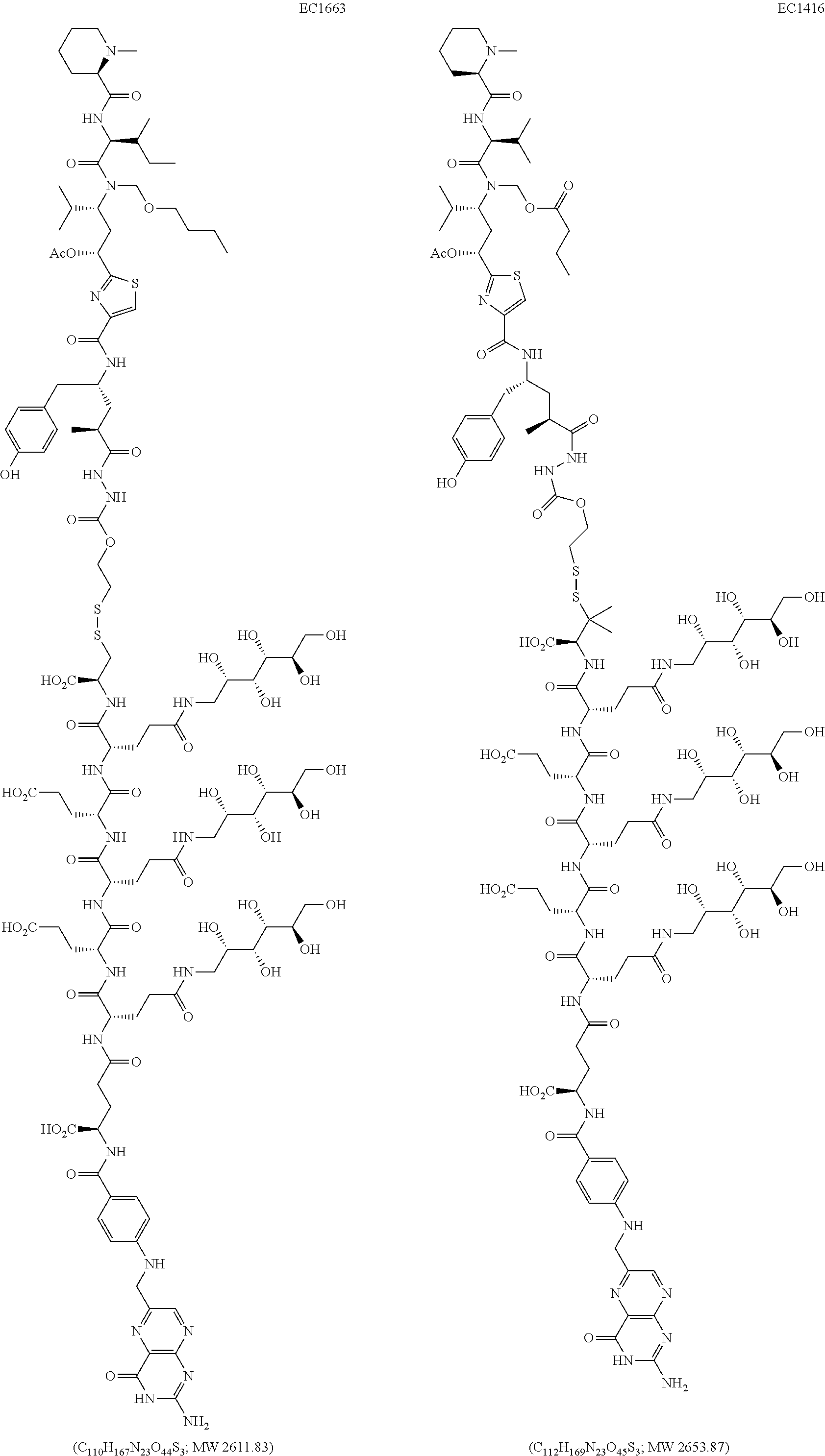

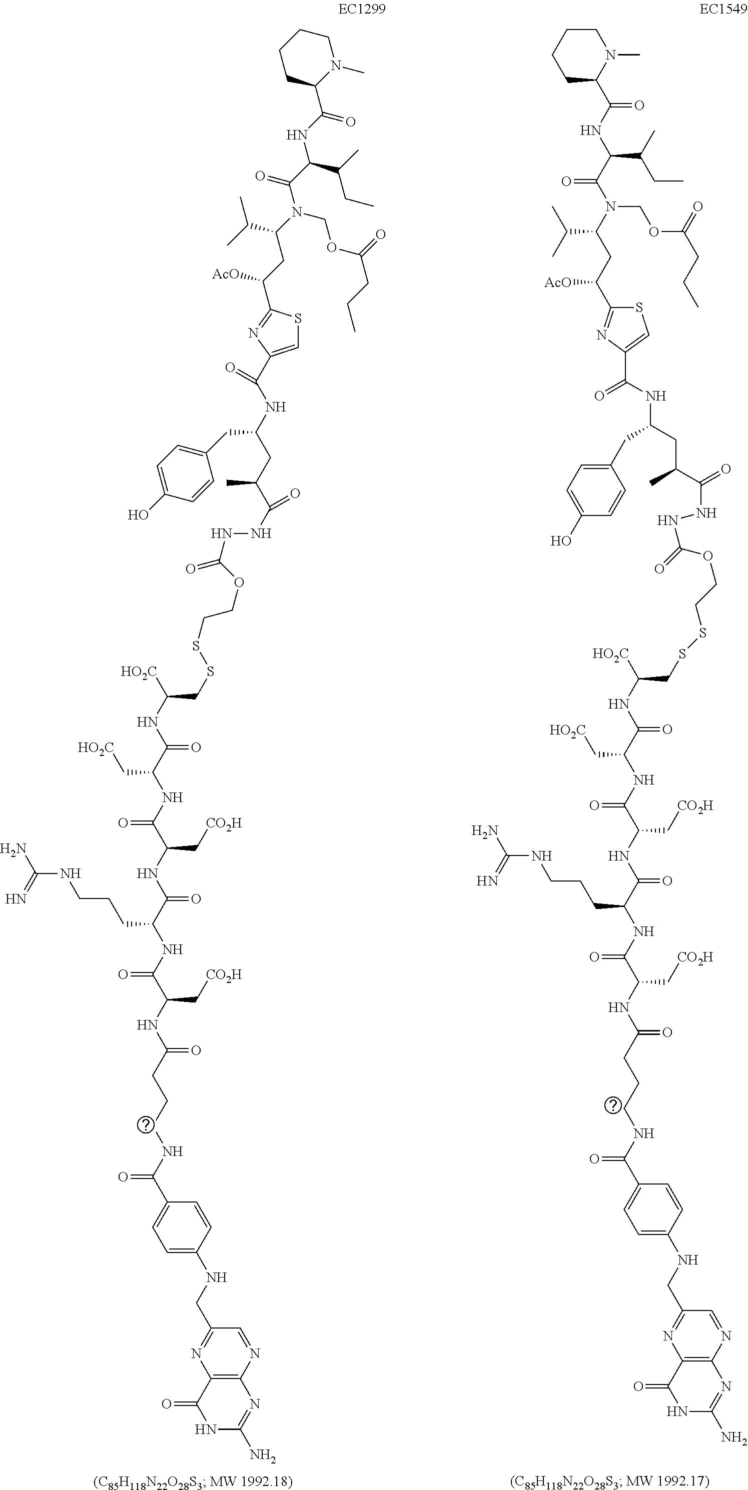

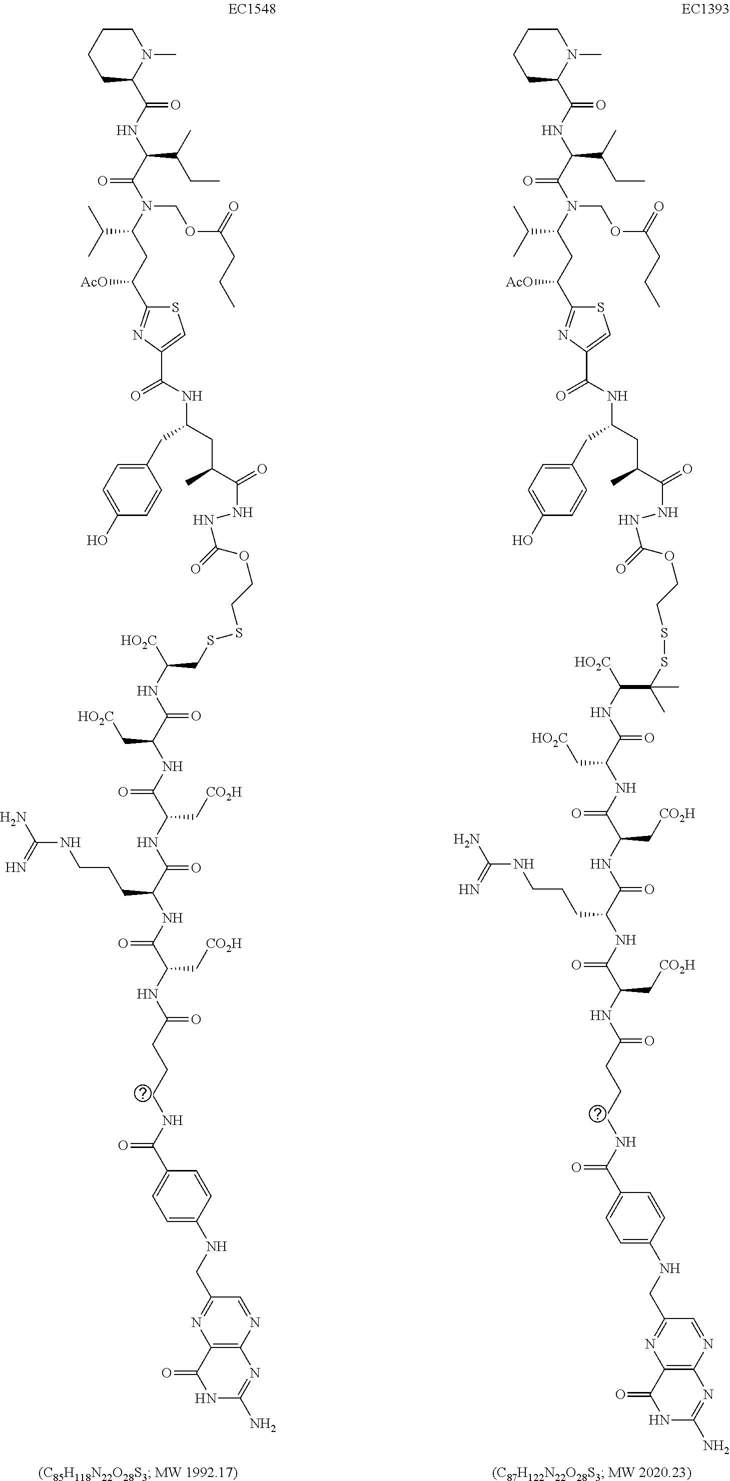

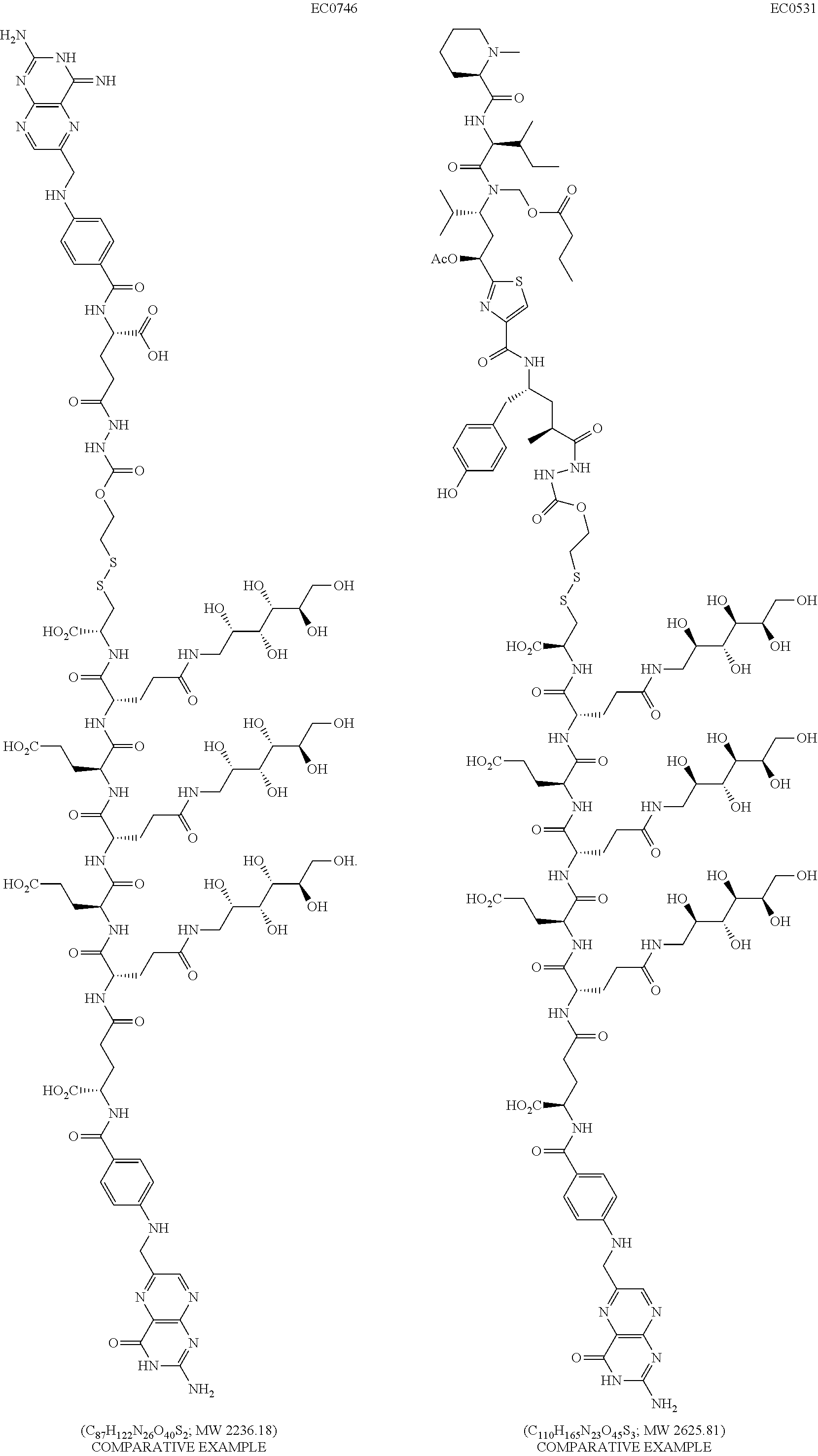

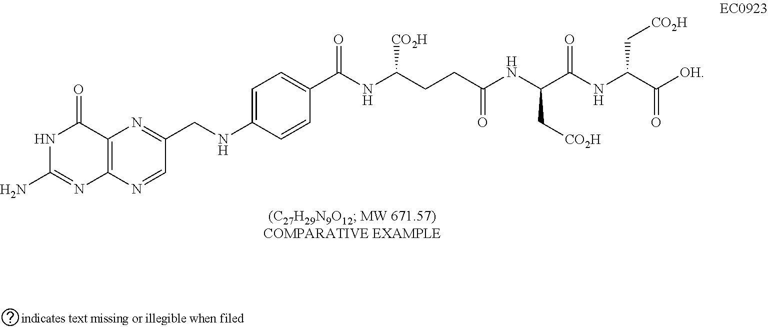 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

### Method Examples

General. The following abbreviations are used herein: partial response (PR); complete response (CR), three times per week (M/W/F) (TIW).

Method.

Relative Affinity Assay. The affinity for folate receptors (FRs) relative to folate was determined according to a previously described method (Westerhof, G. R., J. H. Schornagel, et al. (1995) Mol. Pharm. 48: 459-471) with slight modification. Briefly, FR-positive KB cells were heavily seeded into 24-well cell culture plates and allowed to adhere to the plastic for 18 h. Spent incubation media was replaced in designated wells with folate-free RPMI (FFRPMI) supplemented with 100 nM ³H-folic acid in the absence and presence of increasing concentrations of test article or folic acid. Cells were incubated for 60 min at 37° C. and then rinsed 3 times with PBS, pH 7.4. Five hundred microliters of 1% SDS in PBS, pH 7.4, were added per well. Cell lysates were then collected and added to individual vials containing 5 mL of scintillation cocktail, and then counted for radioactivity. Negative control tubes contained only the ³H-folic acid in FFRPMI (no competitor). Positive control tubes contained a final concentration of 1 mM folic acid, and CPMs measured in these samples (representing non-specific binding of label) were subtracted from all samples. Notably, relative affinities were defined as the inverse molar ratio of compound required to displace 50% of ³H-folic acid bound to the FR on KB cells, and the relative affinity of folic acid for the FR was set to 1.

Method.

Inhibition of Cellular DNA Synthesis. The compounds described herein were evaluated using an in vitro cytotoxicity assay that predicts the ability of the drug to inhibit the growth of folate receptor-positive KB cells. The compounds were comprised of folate linked to a respective chemotherapeutic drug, as prepared according to the protocols described herein. The KB cells were exposed for up to 7 h at 37° C. to the indicated concentrations of folate-drug conjugate in the absence or presence of at least a 100-fold excess of folic acid. The cells were then rinsed once with fresh culture medium and incubated in fresh culture medium for 72 hours at 37° C. Cell viability was assessed using a ³H-thymidine incorporation assay. For compounds described herein, dose-dependent cytotoxicity was generally measurable, and in most cases, the IC₅₀ values (concentration of drug conjugate required to reduce ³H-thymidine incorporation into newly synthesized DNA by 50%) were in the low nanomolar range. Furthermore, the cytotoxicities of the conjugates were reduced in the presence of excess free folic acid, indicating that the observed cell killing was mediated by binding to the folate receptor.

Method.

In vitro test against the various cancer cell lines. IC50 values were generated for various cell lines and the results are shown in the table below. Cells are heavily seeded in 24-well Falcon plates and allowed to form nearly confluent monolayers overnight. Thirty minutes prior to the addition of the test compound, spent medium is aspirated from all wells and replaced with fresh folate-deficient RPMI medium (FFRPMI). A subset of wells are designated to receive media containing 100 μM folic acid. The cells in the designated wells are used to determine the targeting specificity. Without being bound by theory it is suggested that the cytotoxic activity produced by test compounds in the presence of excess folic acid, i.e. where there is competition for FR binding, corresponds to the portion of the total activity that is unrelated to FR-specific delivery. Following one rinse with 1 mL of fresh FFRPMI containing 10% heat-inactivated fetal calf serum, each well receives 1 mL of medium containing increasing concentrations of test compound (4 wells per sample) in the presence or absence of 100 μM free folic acid as indicated. Treated cells are pulsed for 2 h at 37° C., rinsed 4 times with 0.5 mL of media, and then chased in 1 mL of fresh medium up to 70 h. Spent medium is aspirated from all wells and replaced with fresh medium containing 5 μCi/mL ³H-thymidine. Following a further 2 h 37° C. incubation, cells are washed 3 times with 0.5 mL of PBS and then treated with 0.5 mL of ice-cold 5% trichloroacetic acid per well. After 15 min, the trichloroacetic acid is aspirated and the cell material solubilized by the addition of 0.5 mL of 0.25 N sodium hydroxide for 15 min. A 450 μL aliquot of each solubilized sample is transferred to a scintillation vial containing 3 mL of Ecolume scintillation cocktail and then counted in a liquid scintillation counter. Final tabulated results are expressed as the percentage of ³H-thymidine incorporation relative to untreated controls.

Method.

Human serum stability. Compounds described herein are tested in human serum for stability using conventional protocols and methods.

Method.

Inhibition of Tumor Growth in Mice. Four to seven week-old mice (Balb/c or nu/nu strains) were purchased from Harlan Sprague Dawley, Inc. (Indianapolis, Ind.). Normal rodent chow contains a high concentration of folic acid (6 mg/kg chow); accordingly, mice used were maintained on the folate-free diet (Harlan diet #TD00434) for 1 week before tumor implantation to achieve serum folate concentrations close to the range of normal human serum. For tumor cell inoculation, 1×10⁶ M109 cells (Balb/c strain) or 1×10⁶ KB cells (nu/nu strain) in 100 μL were injected in the subcutis of the dorsal medial area. Tumors were measured in two perpendicular directions every 2-3 days using a caliper, and their volumes were calculated as 0.5×L×W², where L=measurement of longest axis in mm and W=measurement of axis perpendicular to L in mm. Log cell kill (LCK) and treated over control (T/C) values were then calculated according to published procedures (see, e.g., Lee et al., “BMS-247550: a novel epothilone analog with a mode of action similar to paclitaxel but possessing superior antitumor efficacy” Clin Cancer Res 7:1429-1437 (2001); Rose, “Taxol-based combination chemotherapy and other in vivo preclinical antitumor studies” J Natl Cancer Inst Monogr 47-53 (1993)). Dosing solutions were prepared fresh each day in PBS and administered through the lateral tail vein of the mice. Dosing was initiated when the s.c. tumors had an average volume between 50-100 mm³ (t₀), typically 8 days post tumor inoculation (PTI) for KB tumors, and 11 days PTI for M109 tumors.

Method.

Drug Toxicity determinations. Persistent drug toxicity was assessed by collecting blood via cardiac puncture and submitting the serum for independent analysis of blood urea nitrogen (BUN), creatinine, total protein, AST-SGOT, ALT-SGPT plus a standard hematological cell panel at Ani-Lytics, Inc. (Gaithersburg, Md.). In addition, histopathologic evaluation of formalin-fixed heart, lungs, liver, spleen, kidney, intestine, skeletal muscle and bone (tibia/fibula) were conducted by board-certified pathologists at Animal Reference Pathology Laboratories (ARUP; Salt Lake City, Utah).

Method.

General KB Tumor Assay. The anti-tumor activity of the compounds described herein, when administered intravenously (i.v.) to tumor-bearing animals, was evaluated in nu/nu mice bearing subcutaneous KB tumors. Approximately 8 days post tumor inoculation in the subcutis of the right axilla with 1×10⁶ KB cells (average tumor volume at tₒ=50-100 mm³), in mice (5/group) were injected i.v. three times a week (TIW), for 3 weeks with 5 mmol/kg of the drug delivery conjugate or with an equivalent dose volume of PBS (control), unless otherwise indicated. Tumor growth was measured using calipers at 2-day or 3-day intervals in each treatment group. Tumor volumes were calculated using the equation V=a×b²/2, where “a” is the length of the tumor and “b” is the width expressed in millimeters.

Method.

General M109 Tumors Assay. The anti-tumor activity of the compounds described herein, when administered intravenously (i.v.) to tumor-bearing animals, was evaluated in Balb/c mice bearing subcutaneous M109 tumors (a syngeneic lung carcinoma). Approximately 11 days post tumor inoculation in the subcutis of the right axilla with 1×10⁶ M109 cells (average tumor volume at tₒ=60 mm³), mice (5/group) were injected i.v. three times a week (TIW), for 3 weeks with 1500 nmol/kg of the drug delivery conjugate or with an equivalent dose volume of PBS (control). Tumor growth was measured using calipers at 2-day or 3-day intervals in each treatment group. Tumor volumes were calculated using the equation V=a×b²/2, where “a” is the length of the tumor and “b” is the width expressed in millimeters.

Method.

General 4T-1 Tumor Assay. Six to seven week-old mice (female Balb/c strain) were obtained from Harlan, Inc., Indianapolis, Ind. The mice were maintained on Harlan's folate-free chow for a total of three weeks prior to the onset of and during this experiment. Folate receptor-negative 4T-1 tumor cells (1×10⁶ cells per animal) were inoculated in the subcutis of the right axilla. Approximately 5 days post tumor inoculation when the 4T-1 tumor average volume was ˜100 mm³, mice (5/group) were injected i.v. three times a week (TIW), for 3 weeks with 3 μmol/kg of drug delivery conjugate or with an equivalent dose volume of PBS (control), unless otherwise indicated herein. Tumor growth was measured using calipers at 2-day or 3-day intervals in each treatment group. Tumor volumes were calculated using the equation V=a×b²/2, where “a” is the length of the tumor and “b” is the width expressed in millimeters.

Method.

Toxicity as Measured by Weight Loss. The percentage weight change of the mice was determined in mice (5 mice/group) on selected days post-tumor inoculation (PTI), and graphed.

Method.

Adjuvant-Induced Arthritis (AIA) Model. Female Lewis rats were fed a folate-deficient diet (Harlan Teklad, Indianapolis, Ind.) for 9-10 days prior to arthritis induction. The adjuvant-induced arthritis (AIA) was induced by intradermal inoculation (at the base of tail) of 0.5 mg of heat-killed Mycobacteria butyricum (BD Diagnostic Systems, Sparks, Md.) in 100 μL light mineral oil (Sigma). Ten days after arthritis induction, paw edema in rats was assessed using a modified arthritis scoring system: 0=no arthritis; 1=swelling in one type of joint; 2=swelling in two types of joint; 3=swelling in three types of joint; 4=swelling of the entire paw. A total score for each rat is calculated by summarizing the scores for each of the four paws, giving a maximum score of 16 for each rat. On Day 10 post arthritis induction, rats with a total arthritis score of ≧2 were removed from the study and the remaining rats were distributed evenly across the control and treatment groups (n=5 for all groups except that n=2-3 for healthy controls). All treatments started on Day 10 unless mentioned otherwise.

Method.

Collagen-Induced Arthritis (CIA) Model. The collagen-induced arthritis (CIA) was induced in female Lewis rats on folate-deficient diet (Harlan Teklad, Indianapolis, Ind.). On Day 0, rats were immunized with 500 μg of bovine collagen Type II (Chondrex, Redmond, Wash.) formulated with Freund's complete adjuvant. A booster immunization was given on Day 7 with 250 μg of the bovine collagen formulated with Freund's incomplete adjuvant. Arthritis disease was assessed by a qualitative clinical score system described by the manufacturer (Chondrex, Redmond, Wash.): 0=normal, 1=Mild, but definite redness and swelling of the ankle or wrist, or apparent redness and swelling limited to individual digits, regardless of the number of affected digits, 2=Moderate redness and swelling of ankle of wrist, 3=Severe redness and swelling of the entire paw including digits, and 4=Maximally inflamed limb with involvement of multiple joints. On Day 10 post first immunization, rats were distributed evenly (according to the arthritis score) across the control and treatment groups. The CIA rats were given ten consecutive subcutaneous doses of EC0746 and methotrexate on days 10-19. For both drugs, an induction dose (500 nmol/kg) was given on days 10 and 15 and a maintenance dose (100 nmol/kg) was given on days 11-14 and 16-19. The animals in the arthritis control group were left untreated. The arthritis score and animal body weight were recorded five times a week. The result showed that EC0746 was also effective in rats with collagen-induced arthritis. See FIG. 13, Panels A and B.

Method.

Animal Experimental Autoimmune Uveitis Model. Experimental autoimmune uveitis (EAU) was induced in female Lewis rats maintained on a folate-deficient diet (Harlan Teklad, Indianapolis, Ind.). On Day 0, the animals were immunized subcutaneously with 25 μg of bovine S-Ag PDSAg peptide formulated with Freund's incomplete adjuvant containing 0.5 mg of M. Tuberculosis H37Ra. Purified pertussis toxin (PT) was given at a dosage of 1 μg per animal on the same day via intraperitoneal injection. The severity of uveitis in each eye was assessed by a qualitative visual score system: 0=No disease, eye is translucent and reflects light (red reflex); 0.5 (trace)=Dilated blood vessels in the iris, 1=Engorged blood vessels in iris, abnormal pupil contraction; 2=Hazy anterior chamber, decreased red reflex; 3=Moderately opaque anterior chamber, but pupil still visible, dull red reflex; and 4=Opaque anterior chamber and obscured pupil, red reflex absent, proptosis. This assessment yields a maximum uveitis score of 8 per animal. FIG. 34 shows images the eyes of an animal (upper right) with severe uveitis on its right eye (bottom) and a healthy eye (upper right).

Method.

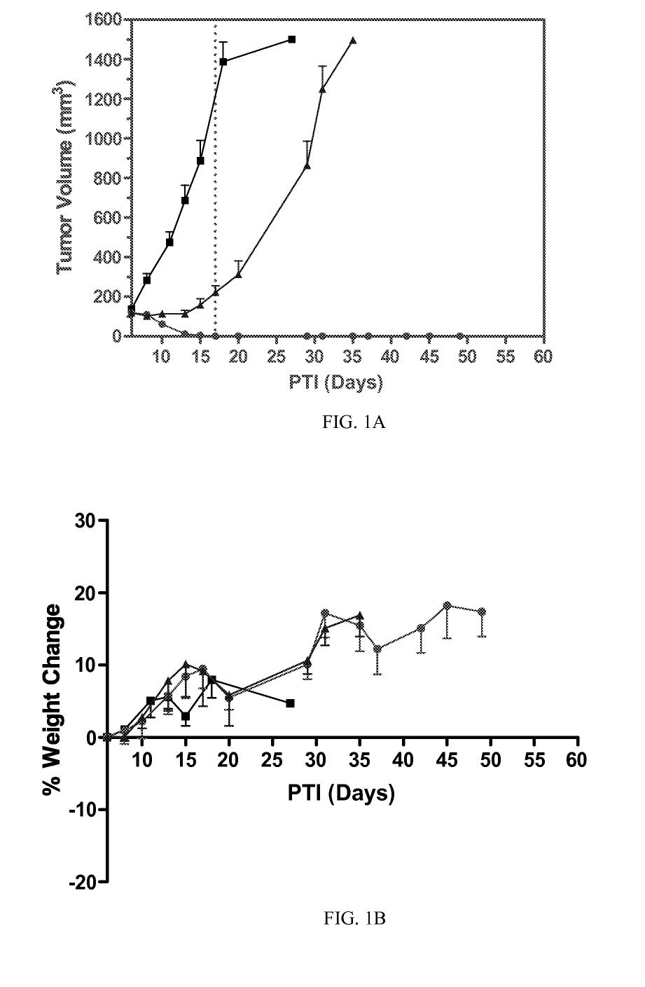

In vivo activity against tumors. Compounds described herein show high potency and efficacy against KB tumors in nu/nu mice. Compounds described herein show specific activity against folate receptor expressing tumors, with low host animal toxicity. For example, EC1456 shows a complete response in 4/4 test animals when administered intravenously at 1 μmol/kg TIW, 2 wk. EC1456 also shows specific activity mediated by the folate receptor as evidenced being competable with excess EC0923 (50 or 100 μmol/kg), as shown in FIG. 1A. EC1456 does not show any evidence of whole animal toxicity, as shown in FIG. 1B.

Method.

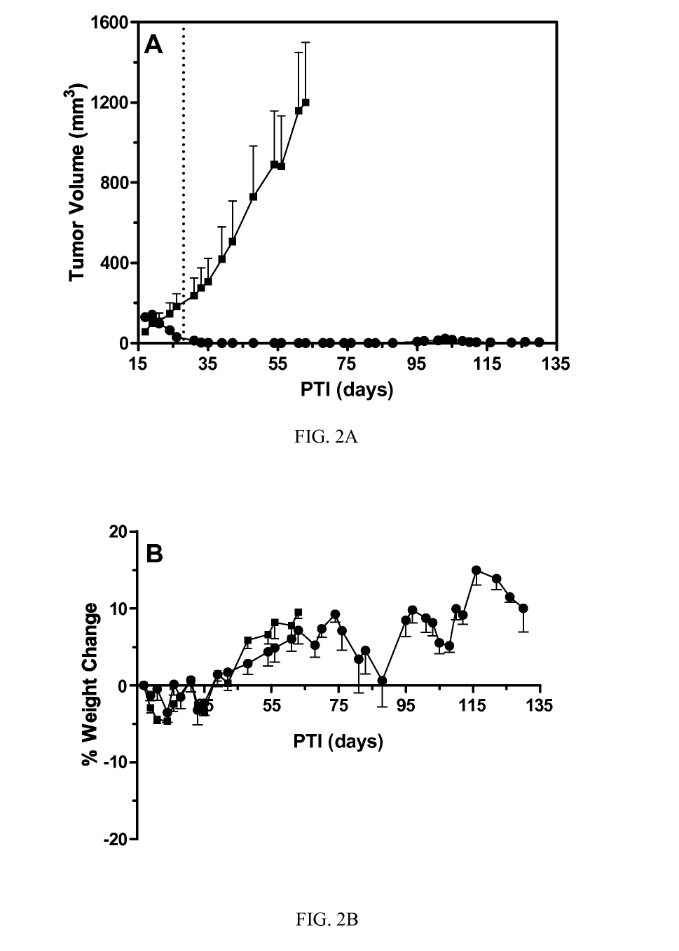

Triple negative breast cancer (TNBC) is a subtype characterized by lack of gene expression for estrogen, progesterone and Her2/neu. TNBC is difficult to treat, and its death rate is disproportionately higher than for any other subtype of breast cancer. When tested against an established triple negative FR-positive subcutaneous MDA-MB-231 breast cancer xenograft, EC1456 was found to be highly active at 2 μmmol/kg intravenous dose administered on a three times per week, 2 consecutive week schedule produced 4 of 5 cures, as shown in FIG. 2A. The anti-tumor activity was not accompanied by significant weight loss in the test animals, as shown in FIG. 2B.

Method.

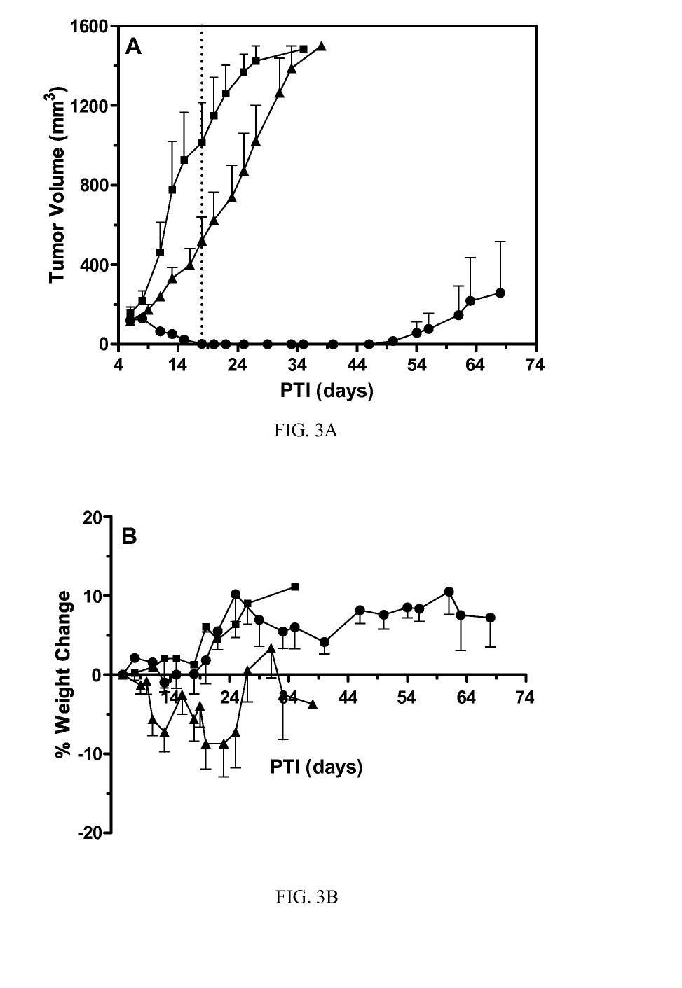

A human cisplatin-resistant cell line was created by culturing FR-positive KB cells in the presence of increasing cisplatin concentrations (100→2000 nM; over a>12 month period). The cisplatin-resistant cells, labeled as KB-CR2000 cells, were found to be tumorigenic, and they retained their FR expression status in vivo. KB-CR tumors were confirmed to be resistant to cisplatin therapy since treatment with a high, toxic dose (average weight loss of 10.3%), as shown in FIG. 3B, of cisplatin produced no PRs, as shown in FIG. 3A. In contrast, EC1456 was found to be very active against KB-CR tumors, where ⅘ cures and ⅕ complete responses were observed. Furthermore, unlike cisplatin, EC1456 did not cause any weight loss in this cohort of mice.

Method.

The therapeutic performance of unconjugated tubulysin B and TubBH drugs was evaluated against the human KB tumor model, and anti-tumor as well as body weight changes were compared to that produced by EC1456. EC1456 produced dose responsive anti-tumor activity against this model. Complete responses were observed under treatment conditions that produced little to no weight loss. In contrast, both unconjugated tubulysin-based drugs failed to yield any anti-tumor response, even when very toxic doses were administered to the mice.

| | | | | | | | Toxicity | |
|---|---|---|---|---|---|---|---|---|
| | | | | | | | | Avg. |
| Test | Dose level | | Dose | PR | CR | Cures | Deaths | Weight |
| Article | (μmol/kg) | | Schedule | (%) | (%) | (%) | (%) | Loss |
| EC1456 | 0.5 | | TIW, 3 weeks | 60 | 0 | 0 | 0 | <5%* |
| | 0.67 | | TIW, 2 weeks | 60 | 20 | 0 | 0 | <2% |
| | 1.0 | | TIW, 2 weeks | 40 | 0 | 60 | 0 | <1.5% |
| | 2.0 | | TIW, 2 weeks | 0 | 0 | 100 | 0 | <3% |
| Tubulysin B | 0.1 | (4 doses) | TIW, 2 weeks | 0 | 0 | 0 | 100 | >20% |
| | 0.2 | (3 doses) | TIW, 2 weeks | 0 | 0 | 0 | 100 | >18% |
| | 0.5 | (1 dose) | TIW, 2 weeks | 0 | 0 | 0 | 100 | >15% |
| TubBH | 0.5 | | TIW, 2 weeks | 0 | 0 | 0 | 0 | <5.5% |
| | 0.75 | | TIW, 2 weeks | 0 | 0 | 0 | 20 | >10% |
| | 1.0 | (2 doses)¹ | TIW, 2 weeks | 0 | 0 | 0 | 20 | >15% |

*Untreated control group had an average weight loss of 2.4%
¹Group received only 2 doses due to toxicity.

These results confirm that despite tubulysin B and TubBH being highly cytotoxic to cells in culture (typical IC₅₀˜1 nM), both agents yielded dose-limiting toxicities in mice at levels that did not produce measurable anti-tumor effect. In contrast, the folate-targeted form of TubBH (EC1456) produced anti-tumor responses without significant toxicity to mice bearing well-established human tumor xenografts.

Method.

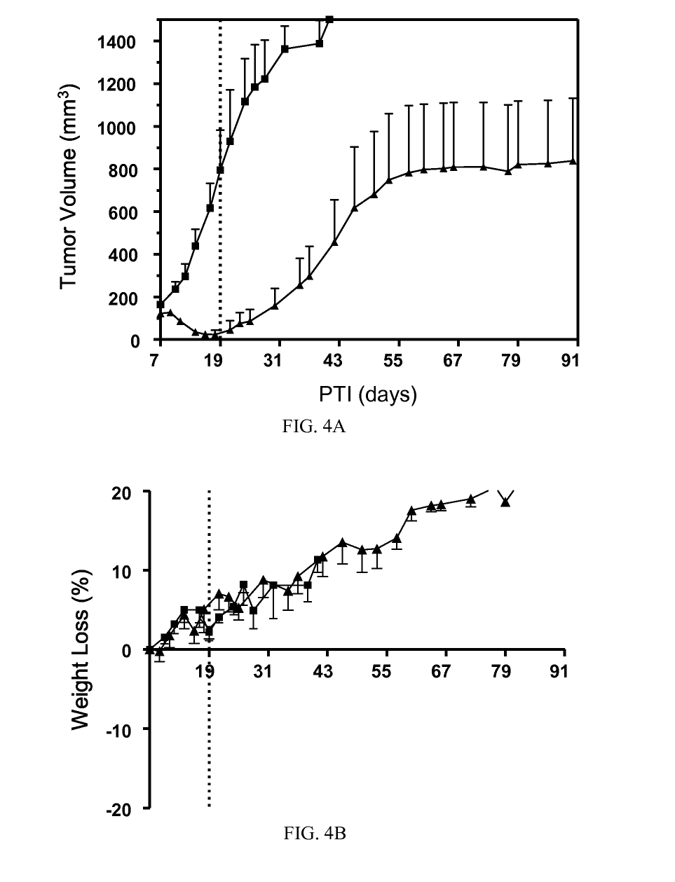

EC1663 is efficacious against against KB tumors in nu/nu mice and shows 4/4 partial responses in test animals when administered intravenously at 0.5 μmol/kg TIW, 2 wk compared to untreated (PBS) controls, as shown in FIG. 4A. EC1663 does not show any evidence of whole animal toxicity compared to PBS control, as shown in FIG. 4B.

Method.

Maximum tolerated dose (MTD). Compounds described herein show high MTDs, which are improved over compounds that do not have linkers comprising one or more unnatural amino acids. For Example, EC1456 has a MTD of at least 0.51 μmol/kg and EC0531 has a MTD of 0.33 μmol/kg, a 65% improvement when administered by i.v., BIW, 2 wks in female Sprague-Dawley rats. Histopathologic changes were not observed with doses of EC1456 at or below the MTD.

Method.

Folate receptor expressing cells. Compounds described herein show high activity for folate receptor expressing cells. Compounds described herein do not show significant binding to folate receptor negative cells. EC1456 was evaluated from 0.1-100 nM.

Activity of EC1456 in Various FR+ and FR− Cell Lines

| | | FR | EC1456 | Competable |
|---|---|---|---|---|
| Cell Line | Origin | Status | Activity* (IC₅₀) | up to 100 nM |
| KB | Human cervical carcinoma | +++ | 2.3 nM | Yes |
| NCI/ADR-RES-Cl₂ | Human ovarian carcinoma | ++ | 1.4 nM | Yes |
| IGROV1 | Human ovarian adenocarcinoma | + | 0.72 nM | Yes |
| MDA-MB-231 | Human breast adenocarcinoma | + | 0.47 nM | Yes |
| | (triple negative) | | | |
| A549 | Human lung carcinoma | − | Inactive | |
| H23 | Human lung adenocarcinoma | − | Inactive | |
| HepG2 | Human hepatocellular carcinoma | − | Inactive | |
| AN3CA | Human endometrial | − | Inactive | |
| | adenocarcinoma | | | |
| LNCaP | Human prostate adenocarcinoma | − | ~850 nM | |

*EC1456 activity was evaluated from 0.1-100 nM

Method.

Compounds described herein exhibit potent in vitro activity against pathogenic cells, such as KB cells. Compounds described herein exhibit greater specificity for the folate receptor compared to compounds that do not include at least one unnatural amino acid. For Example, EC1456 exhibits ca. 1000-fold specificity for the folate receptor as determined by folic acid competition (Specificity=difference in IC₅₀ between competed group and non-competed group), and a 4-fold improvement in specificity compared to EC0531, which does not include a linker L having an unnatural amino acid.

Method.

Compounds described herein exhibit high folate receptor affinity compared to folic acid (relative affinity=1) in 10% serum/FDRPMI, potent in vitro activity, potent in vivo activity, specificity for the folate receptor, and a sufficiently high therapeutic index over unconjugated tubulysin

| | | | | | | Therapeutic |
|---|---|---|---|---|---|---|
| | | In vitro | 50% | In vitro | | index over |
| | Relative | IC50 | competition | specificity | In vivo | parent |
| Example | Affinity | (nM) | (nM) | (fold) | activity | tubulysin |
| EC1299 | 0.29 | 0.9 | 700 | 778 | Complete | Yes |
| | | | | | response | |
| EC1393 | 0.25 | 2.2 | 600 | 300 | Not | Not tested |
| | | | | | tested | |
| EC1456 | 0.27 | 1.5 | 1416 | 944 | Complete | Yes |
| | | | | | response | |
| EC1548 | 0.23 | 4.4 | 350 | 78 | Complete | Yes |
| | | | | | response | |
| EC1549 | 0.90 | 4.5 | 350 | 78 | Complete | Yes |
| | | | | | response | |
| EC1586 | 0.56 | Not | Not tested | Not tested | Not | Not tested |
| | | tested | | | tested | |
| EC0531 | Not tested | 1.5 | 355 | 237 | Complete | Yes |
| (comparator | | | | | response | |
| example) | | | | | | |

Method.

Compounds described herein are more stable in plasma than compounds that do not have a linker comprising at least one unnatural amino acid. EC1496 releases ca. 50% less drug than the comparative example EC0746 after a AC dose in rats.

What is claimed is:

1. A compound of the formula BLD_{X}, or a pharmaceutically acceptable salt thereof, wherein B is a cell surface receptor targeting ligand, D is in each instance an independently selected drug, x is an integer selected from 1, 2, 3, 4 and 5; and L is a releasable polyvalent linker comprising one or more unnatural amino acids; and where B iscovalently attached to L, and L is covalently attached to each of D; and
where that the compound is not of the formula 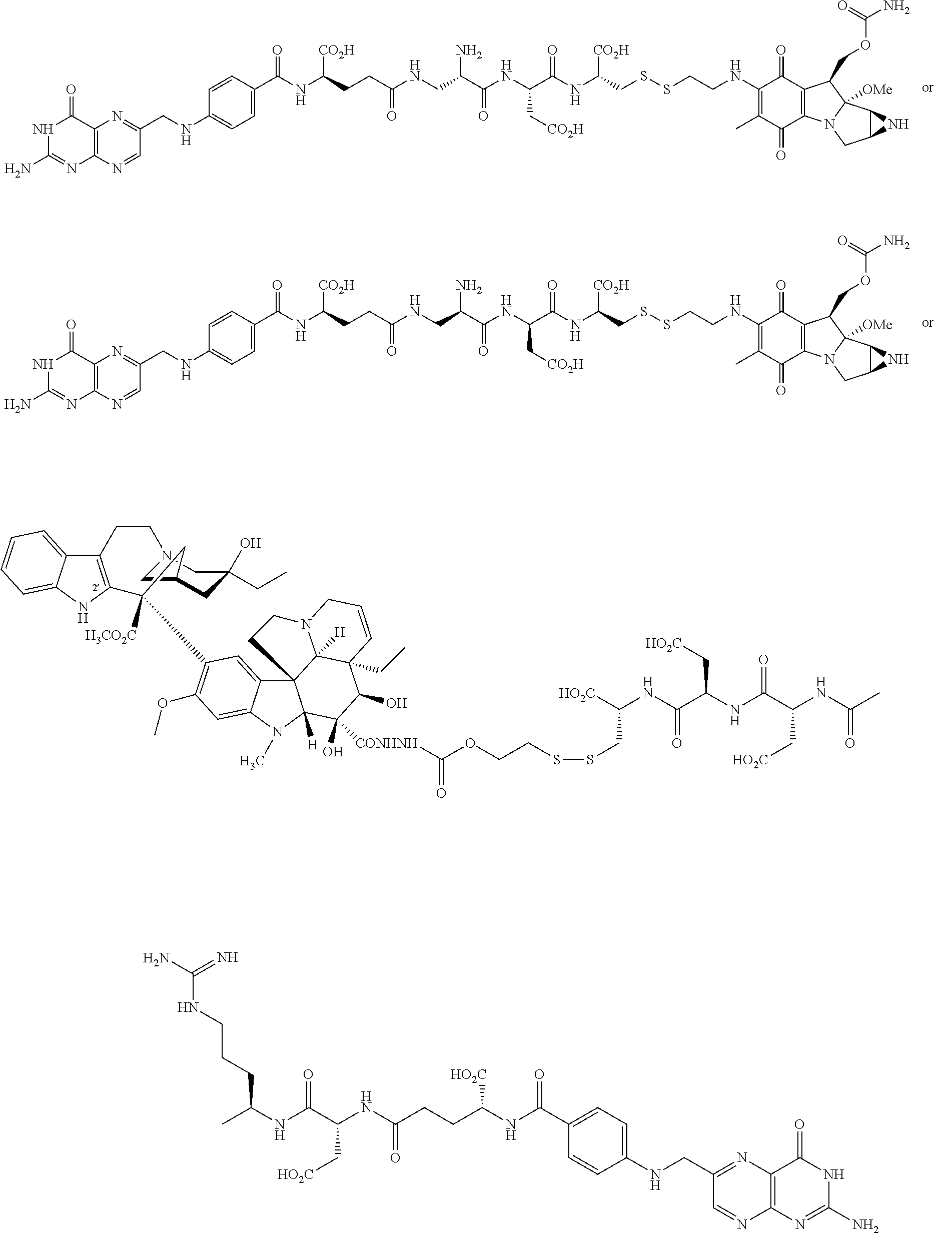
or a pharmaceutically acceptable salt thereof.

2. The compound of claim 1 wherein at least one unnatural amino acid has the D-configuration.

3. The compound of claim 1 wherein at least one unnatural amino acid is selected from D-alanine, D-aspartic acid, D-asparagine, D-cysteine, D-glutamic acid, D-phenylalanine, D-histidine, D-isoleucine, D-lysine, D-leucine, D-methionine, D-proline, D-glutamine, D-arginine, D-serine, D-threonine, D-valine, D-tryptophan, D-tyrosine, and D-ornithine, or a derivative thereof

4. The compound of claim 1 wherein at least one unnatural amino acid is selected from D-aspartic acid, D-asparagine, D-cysteine, D-glutamic acid, D-histidine, D-lysine, D-methionine, D-glutamine, D-arginine, D-serine, D-threonine, D-tryptophan, D-tyrosine, and D-ornithine, or a derivative thereof.

5. The compound of claim 1 wherein at least one unnatural amino acid is selected from D-aspartic acid, D-asparagine, D-cysteine, D-glutamic acid, D-histidine, D-lysine, D-glutamine, D-arginine, D-serine, D-threonine, D-tryptophan, and D-ornithine, or a derivative thereof.

6. The compound of claim 1 wherein at least one unnatural amino acid is selected from D-aspartic acid, D-cysteine, D-glutamic acid, D-lysine, D-arginine, D-serine, and D-ornithine, or a derivative thereof.

7. The compound of claim 1 wherein L comprises two or more unnatural amino acids.

8. The compound of claim 1 wherein L comprises three or more unnatural amino acids.

9. The compound of claim 1 wherein L comprises four or more unnatural amino acids.

10. The compound of claim 1 wherein L further comprises one or more disulfides.

11. The compound of claim 1 wherein at least one disulfide comprises D-cysteinyl.

12. The compound of claim 1 wherein L further comprises one or more divalent hydrophilic radicals.

13. The compound of claim 1 wherein L further comprises two or more divalent hydrophilic radicals.

14. The compound of claim 1 wherein L further comprises three or more divalent hydrophilic radicals.

15. The compound of claim 1 wherein L further comprises four or more divalent hydrophilic radicals.

16. The compound of claim 1 wherein L further comprises one or more divalent polyoxy radicals.

17. The compound of claim 1 wherein L further comprises two or more divalent polyoxy radicals.

18. The compound of claim 1 wherein L further comprises three or more divalent polyoxy radicals.

19. The compound of claim 1 wherein L further comprises four or more divalent polyoxy radicals.

20. The compound of claim 1 wherein L further comprises one or more divalent polyhydroxy radicals.
